SEQUENCE LISTING (74 sequences)

SEQ ID NO: 1 - DNA, 20 bp, synthetic construct
  source 1..20: mol_type = other DNA
agcagcacctgaagaaggtc

SEQ ID NO: 2 - DNA, 20 bp, synthetic construct
  source 1..20: mol_type = other DNA
tcaatatcagggagcccaag

SEQ ID NO: 3 - DNA, 19 bp, synthetic construct
  source 1..19: mol_type = other DNA
gagattccagtgtgcctgt

SEQ ID NO: 4 - DNA, 20 bp, synthetic construct
  source 1..20: mol_type = other DNA
gtccaatagcccaggatgtg

SEQ ID NO: 5 - DNA, 20 bp, synthetic construct
  source 1..20: mol_type = other DNA
caaagtgacagtgggtgtgg

SEQ ID NO: 6 - DNA, 19 bp, synthetic construct
  source 1..19: mol_type = other DNA
ccaggtccttcactgtctc

SEQ ID NO: 7 - DNA, 20 bp, synthetic construct
  source 1..20: mol_type = other DNA
gaagcagttcctggacttcg

SEQ ID NO: 8 - DNA, 20 bp, synthetic construct
  source 1..20: mol_type = other DNA
accaattgaacggatggtgt

SEQ ID NO: 9 - DNA, 20 bp, synthetic construct
  source 1..20: mol_type = other DNA
gccggctttacgttcacctc

SEQ ID NO: 10 - DNA, 20 bp, synthetic construct
  source 1..20: mol_type = other DNA
gttgaagcaccactgggcac

SEQ ID NO: 11 - DNA, 20 bp, synthetic construct
  source 1..20: mol_type = other DNA
cttcactgtcgtgtcgctgt

SEQ ID NO: 12 - DNA, 20 bp, synthetic construct
  source 1..20: mol_type = other DNA
ccaggacccacttcaaagaa

SEQ ID NO: 13 - DNA, 20 bp, synthetic construct
  source 1..20: mol_type = other DNA
aagaacgtgaagctccctga

SEQ ID NO: 14 - DNA, 20 bp, synthetic construct
  source 1..20: mol_type = other DNA
aatataggggatgggcttgg

SEQ ID NO: 15 - DNA, 20 bp, synthetic construct
  source 1..20: mol_type = other DNA
ggtgcacaacgggatagagt

SEQ ID NO: 16 - DNA, 20 bp, synthetic construct
  source 1..20: mol_type = other DNA
ccatcggtgtcttggaactt

SEQ ID NO: 17 - DNA, 20 bp, synthetic construct
  source 1..20: mol_type = other DNA
tggaaaggatctgggaagtg

SEQ ID NO: 18 - DNA, 19 bp, synthetic construct
  source 1..19: mol_type = other DNA
cctggggtcaagatccata

SEQ ID NO: 19 - DNA, 20 bp, synthetic construct
  source 1..20: mol_type = other DNA
ctggatcgacacccagagat

SEQ ID NO: 20 - DNA, 20 bp, synthetic construct
  source 1..20: mol_type = other DNA
cgatcacgatgaagcgacta

SEQ ID NO: 21 - DNA, 18 bp, synthetic construct
  source 1..18: mol_type = other DNA
tgaggagacaccgcccac

SEQ ID NO: 22 - DNA, 25 bp, synthetic construct
  source 1..25: mol_type = other DNA
caacatcgatttcttcctcatcttc

SEQ ID NO: 23 - DNA, 20 bp, synthetic construct
  source 1..20: mol_type = other DNA
ctaccagcgacttgccatca

SEQ ID NO: 24 - DNA, 20 bp, synthetic construct
  source 1..20: mol_type = other DNA
atcatagatggcgagcatcc

SEQ ID NO: 25 - DNA, 22 bp, synthetic construct
  source 1..22: mol_type = other DNA
gagggctaggtctttctggaag

SEQ ID NO: 26 - DNA, 22 bp, synthetic construct
  source 1..22: mol_type = other DNA
ccacagtgaaatctcgttgaga

SEQ ID NO: 27 - DNA, 20 bp, synthetic construct
  source 1..20: mol_type = other DNA
gccagaagcccccaagaaac

SEQ ID NO: 28 - DNA, 20 bp, synthetic construct
  source 1..20: mol_type = other DNA
cgacatgctcggcctcatag

SEQ ID NO: 29 - DNA, 19 bp, synthetic construct
  source 1..19: mol_type = other DNA
actccttgggcctttgctg

SEQ ID NO: 30 - DNA, 23 bp, synthetic construct
  source 1..23: mol_type = other DNA
ttctggaacaggaccactgaagt

SEQ ID NO: 31 - DNA, 20 bp, synthetic construct
  source 1..20: mol_type = other DNA
cgtgagctacccggacaatc

SEQ ID NO: 32 - DNA, 23 bp, synthetic construct
  source 1..23: mol_type = other DNA
aaagttgagctcgtagcacaagg

SEQ ID NO: 33 - DNA, 21 bp, synthetic construct
  source 1..21: mol_type = other DNA
gaacagatccagtggttgcag

SEQ ID NO: 34 - DNA, 21 bp, synthetic construct
  source 1..21: mol_type = other DNA
ggcattatgagacatccccac

SEQ ID NO: 35 - DNA, 22 bp, synthetic construct
  source 1..22: mol_type = other DNA
ggagaacctaagtctgcgcact

SEQ ID NO: 36 - DNA, 18 bp, synthetic construct
  source 1..18: mol_type = other DNA
tccctccactgccacagg

SEQ ID NO: 37 - DNA, 22 bp, synthetic construct
  source 1..22: mol_type = other DNA
tatgcttcttcgtgcagcagtt

SEQ ID NO: 38 - DNA, 20 bp, synthetic construct
  source 1..20: mol_type = other DNA
gctgccacacgctcctctag

SEQ ID NO: 39 - DNA, 22 bp, synthetic construct
  source 1..22: mol_type = other DNA
gccaaacagctcacggttgtag

SEQ ID NO: 40 - DNA, 23 bp, synthetic construct
  source 1..23: mol_type = other DNA
ccagatgaagcattgagccactg

SEQ ID NO: 41 - DNA, 20 bp, synthetic construct
  source 1..20: mol_type = other DNA
atgccagagaaggtcacatg

SEQ ID NO: 42 - DNA, 20 bp, synthetic construct
  source 1..20: mol_type = other DNA
acacattccccttcaccttc

SEQ ID NO: 43 - DNA, 21 bp, synthetic construct
  source 1..21: mol_type = other DNA
gctcgctaccagaaatcctac

SEQ ID NO: 44 - DNA, 23 bp, synthetic construct
  source 1..23: mol_type = other DNA
catccaataccaacacataaggc

SEQ ID NO: 45 - DNA, 20 bp, synthetic construct
  source 1..20: mol_type = other DNA
ctggaacggtgaaggtgaca

SEQ ID NO: 46 - DNA, 23 bp, synthetic construct
  source 1..23: mol_type = other DNA
aagggacttcctgtaacaatgca

SEQ ID NO: 47 - DNA, 25 bp, synthetic construct
  source 1..25: mol_type = other DNA
gtctagaaaaacctgccaaatatga

SEQ ID NO: 48 - DNA, 23 bp, synthetic construct
  source 1..23: mol_type = other DNA
ctgttgaagtcagaggagaccac

SEQ ID NO: 49 - DNA, 23 bp, synthetic construct
  source 1..23: mol_type = other DNA
tgcaggggcctatgtccatatgg

SEQ ID NO: 50 - DNA, 18 bp, synthetic construct
  source 1..18: mol_type = other DNA
gcactaccagctaactca

SEQ ID NO: 51 - DNA, 20 bp, synthetic construct
  source 1..20: mol_type = other DNA
cgttgctgtgtggttattgg

SEQ ID NO: 52 - DNA, 20 bp, synthetic construct
  source 1..20: mol_type = other DNA
tcaatcctgcctctcctgat

SEQ ID NO: 53 - DNA, 23 bp, synthetic construct
  source 1..23: mol_type = other DNA
tgcaggggcctatgtccatatgg

SEQ ID NO: 54 - RNA, 20 nt, synthetic construct
  source 1..20: mol_type = other RNA
aaggcactgcattgatcttt

SEQ ID NO: 55 - DNA, 23 bp, synthetic construct
  source 1..23: mol_type = other DNA
tgcaggggcctatgtccatatgg

SEQ ID NO: 56 - DNA, 21 bp, synthetic construct
  source 1..21: mol_type = other DNA
gcactaccagaggctaactca

SEQ ID NO: 57 - DNA, 20 bp, synthetic construct
  source 1..20: mol_type = other DNA
cgttgctgtgtggttattgg

SEQ ID NO: 58 - DNA, 20 bp, synthetic construct
  source 1..20: mol_type = other DNA
tcaatcctgcctctcctgat

SEQ ID NO: 59 - DNA, 23 bp, synthetic construct
  source 1..23: mol_type = other DNA
tgcaggggcctatgtccatatgg

SEQ ID NO: 60 - DNA, 50 bp, Homo sapiens
  source 1..50: mol_type = genomic DNA
gtttgtggcggctgcaggggcctatgtccatatggtcactcatttcattc

SEQ ID NO: 61 - DNA, 50 bp, Homo sapiens
  source 1..50: mol_type = genomic DNA
gaatgaaatgagtgaccatatggacataggcccctgcagccgccacaaac

SEQ ID NO: 62 - RNA, 20 nt, synthetic construct
  source 1..20: mol_type = other RNA
tgcaggggccatacaggata

SEQ ID NO: 63 - DNA, 43 bp, Homo sapiens
  source 1..43: mol_type = genomic DNA
gtttgtggcggctgcaggggcctaatggtcactcatttcattc

SEQ ID NO: 64 - DNA, 40 bp, Homo sapiens
  source 1..40: mol_type = genomic DNA
gtttgtggcggctgcaggggcctatgtcctcatttcattc

SEQ ID NO: 65 - DNA, 49 bp, Homo sapiens
  source 1..49: mol_type = genomic DNA
gtttgtggcggctgcaggggcctatgtcatatggtcactcatttcattc

SEQ ID NO: 66 - DNA, 33 bp, Homo sapiens
  source 1..33: mol_type = genomic DNA
gtttgtggcggctgcaggtcactcatttcattc

SEQ ID NO: 67 - DNA, 49 bp, Homo sapiens; identical to SEQ ID NO: 65
  source 1..49: mol_type = genomic DNA

SEQ ID NO: 68 - DNA, 48 bp, Homo sapiens
  source 1..48: mol_type = genomic DNA
gtttgtggcggctgcaggggcctatgtatatggtcactcatttcattc

SEQ ID NO: 69 - DNA, 43 bp, Homo sapiens; identical to SEQ ID NO: 63
  source 1..43: mol_type = genomic DNA

SEQ ID NO: 70 - DNA, 40 bp, Homo sapiens; identical to SEQ ID NO: 64
  source 1..40: mol_type = genomic DNA

SEQ ID NO: 71 - protein, 237 aa, Homo sapiens
MNIFDRKINFDALLKFSHITPSTQQHLKKVYASFALCMFVAAAGAYVHMVTHFIQAGLLSALGSLILMIWLMATPHSHETEQKRLGLLAGFAFLTGVGLGPALEFCIAVNPSILPTAFMGTAMIFTCFTLSALYARRRSYLFLGGILMSALSLLLLSSLGNVFFGSIWLFQANLYVGLVVMCGFVLFDTQLIIEKAEHGDQDYIWHCIDLFLDFITVFRKLMMILAMNEKDKKKEKK

SEQ ID NO: 72 - protein, 214 aa, Bacillus subtilis
MQATVHESKQSIMQRILTVFVFTLLIATVGLFIGQFVPVALMLPLSILEVAMIILAFWMRRRKAVGYAFVYTFAFVSGITLFPIVSHYASIAGAYVVLEAFGSTFVIFAVLGTIGAKMKKDLSFLWSFLLVAVLALAVVGIFNIFSPLNSAAMMAYSVIGTIVFSLYILYDLNQIKHRHITEDLIPVMALSLYLDFINLFINLLRFFGILSSDD

SEQ ID NO: 73 - protein, 84 aa, Homo sapiens
SSIDRHMYHSLYLKVKGNVFKNKRILMEHIHKLKADKARKKLLADQAEARRSKTKEARKRREERLQAKKEEIIKTLSKEEETKK

SEQ ID NO: 74 - protein, 29 aa, Homo sapiens
SSVITSVKRWQKGQREFKRISRSIRKLQC

(19) United States
(12) Patent Application Publication
(10) Pub. No.: US 2024/0139125 A1
(43) Pub. Date: May 2, 2024

(54) BI-1 ANTAGONISTS AND THEIR USES

(71) Applicant: INDUSTRIAL COOPERATION FOUNDATION JEONBUK NATIONAL UNIVERSITY, Jeonju-si (KR)

(72) Inventor: Han Jung CHAE, Jeonju-si (KR)

(21) Appl. No.: 18/153,896

(22) Filed: Jan. 12, 2023

Related U.S. Application Data
(63) Continuation of application No. PCT/KR2021/008834, filed on Jul. 9, 2021.

(30) Foreign Application Priority Data
Jul. 13, 2020 (KR) 10-2020-0086414
Jul. 7, 2021 (KR) 10-2021-0088876

(51) Int. Cl. A61K 31/136; A61K 31/12; A61K 31/155; A61K 31/166; A61K 31/196; A61K 31/27; A61K 31/277; A61K 31/42; A61K 31/4406; A61K 31/4409; A61K 31/505; A61P 11/06; A61P 29/00; A61P 31/14; A61P 35/00

(52) U.S. Cl. CPC A61K 31/136; A61K 31/12; A61K 31/155; A61K 31/166; A61K 31/196; A61K 31/27; A61K 31/277; A61K 31/42; A61K 31/4406; A61K 31/4409; A61K 31/505; A61P 11/06; A61P 29/00; A61P 31/14; A61P 35/00

(57) ABSTRACT

Proposed is 2E-1-2-aminophenyl-3-3-nitrophenyl-2-propen-1-one (BIA) or its analogues relate to the use of the prevention, treatment and improvement of diseases characterized by the development of abnormal cells or cancer. The BIA and its analogues presented in this disclosure inhibit the calcium-free function of the BI-1 (TMBIM6) gene, thereby reducing binding to mTORC2, and reduce mTORC1 and mTORC2 activity and recruiting ribosomes. It reduces and ultimately inhibits AKT, which has the effect of inhibiting cancer growth.

### CROSS REFERENCE TO RELATED APPLICATIONS

This is a continuation application of International Patent Application No. PCT/KR2021/008834, filed on Jul. 9, 2021, which claims priority to Korean patent application No. KR 10-2020-0086414 filed on Jul. 13, 2020, and KR 10-2021-0088876 filed on Jul. 7, 2021, contents of each of which are incorporated herein by reference in their entireties.

### SEQUENCE LISTING STATEMENT

The present application is being filed along with a Sequence Listing in electronic format. The Sequence Listing is provided as a file entitled “2023-05-03_Sequence_Listing-HNLM022-001C1.txt,” which was created on May 3, 2023, and is 66,910 bytes in size. The information in the electronic format of the Sequence Listing is incorporated herein by reference in its entirety.

### BACKGROUND

### Technical Field

The present disclosure is based on the BI-1 (Bax inhibitor-1; TMBIM6) antagonists, in particular 2E-1-2-aminophenyl-3-3-nitrophenyl-2-propene-1-one and its analogues compounds related to medicinal uses.

### Description of Related Technology

Tumors are the product of uncontrollable disordered cell proliferation caused by an excess of abnormal cells and are separated into malignant tumors when they have destructive proliferative, invasive, and metastatic properties. Three types of treatment are set to treat malignant tumors; radiotherapy, surgical intervention, and chemotherapy, and it is treated through one or a combination of these. Among the cancer treatment methods, chemotherapy is used to treat cancer by disrupting the replication or metabolism of cancer cells, but anticancer drugs as true therapeutics have not yet been developed. Moreover, due to side effects or resistance to anticancer drugs induced by anticancer drugs. Since the effectiveness of anticancer drugs is very low, adjuvant dental drugs are only to help prolong the life of the liver.

### SUMMARY

One aspect is to identify the upstream component of the mTOR signaling path and its associated signaling path and provide its antagonist.

The following terms as used herein have the following meanings unless otherwise specified:

### Definition

As used herein and in the accompanying the claims, the singular forms “one”, “one” and “above” include plurals unless clearly stated otherwise in context.

“Alkyl” alone or as part of another substituent means a fully saturated aliphatic hydrocarbon radical that is a straight or branched chain with a specified number of carbons unless otherwise specified. Or example, “C₁-C₁₀ alkyl” refers to a straight or branched hydrocarbon radical containing 1 to 10 carbon atoms derived by removing one hydrogen atom from a single carbon atom of the parent alkane.

In the context of the present disclosure, unless otherwise specified, the term “alkyl” means “C₁-C₁₀ alkyl”, preferably “C₁-C₅ alkyl”.

“Alkenyl” alone or as part of another substituent means a straight chain or branched chain that may be monounsaturated or polyunsaturated with a specified number of carbons. For example, “C₂-C₈ alkenyl” means an alkenyl radical having 2, 3, 4, 5, 6, 7, or 8 atoms derived by removing one hydrogen atom from a single carbon atom of the parent alkane. In the context of the present disclosure, unless otherwise specified, the term “alkenyl” means “C₂-C₁₀ alkenyl”, preferably “ C₂-C₅ alkenyl.”

“Alkynyl” alone or as part of another substituent means a straight chain or branched hydrocarbon radical that may be monounsaturated or polyunsaturated with a specified number of carbons. For example, “C₂-C₈ alkynyl” means an alkynyl radical having 2 to 8 carbon atoms derived by removing one hydrogen atom from a single carbon atom of the parent alkane. In the context of the present disclosure, unless otherwise specified, the term “alkynyl” means “C₂-C₁₀ alkynyl”, preferably “C₂-C₅ alkynyl”.

“Substitution” refers to the replacement of one or more bonds to carbon (s) or hydrogen (s) by bonds to non-hydrogen and non-carbon atom “substituents”.

In one aspect of the present disclosure, the compound represented by the following formula (1) or a pharmaceutically acceptable salt thereof is provided:

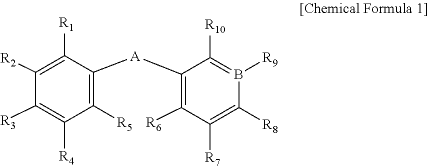

here A is the following formula 1-1, 1-2, or 1-3,

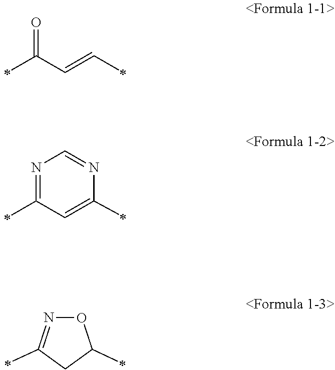

(* of formula 1-1- to 1-3 are connected to the phenyl group of formula 1)
   B and C are C, or N, respectively,
   the R₁ to R₁₀ are respectively H; NH₂; NO2; OH; OR (R is a linear, branched alkyl, alkenyl, or alkynyl of C₁ to C₁₀); halogen atoms; CN; Alkyl halides of C₁ to C₃; a linear, branched alkyl, alkenyl, or alkynyl group of C₁ to C₁₀; —NH—C(O)—ORa (Ra is a linear, branched alkyl, alkenyl, or alkynyl of C₁ to C₁₀ IM); —C(O)—NH—RA (Ra is a linear, branched alkyl, alkenyl, or alkynyl of C₁ to C₁₀); —NH₂HCl; —C(O)OH; And substituents having oxime; One or more selected from the group consisting of,
   if B or C is N, R₈ and R₉ are hydrogen,
   when B or C is C, R₈ and R₉ can be connected to form a phenyl ring, The substituent having the jade core is represented by the following formula 1-4.

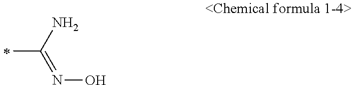

(*of formulas 1-4 is where substituents R₁ to R₁₀ of Formula 1 are substituted)

In one specific embodiment of the present disclosure, the compound of Formula 1 may be C in Formula 1, and A may be Formula 1-1, Formula 1-2 or Formula 1-3.

One of the novel aspects of the present disclosure is that formula 1 of compound A comprises compounds B and C. Additionally, formula 1-1 of compound A and its substituents R8 and R9 may have combined to form a phenyl group.

In one specific embodiment of the present disclosure, the compound of Formula 1 may be B or C of Formula 1 is N, and A may be Formula 1-1.

In one embodiment of the present disclosure, the compound of formula 1 may be selected from the following group:
(1) 2E-1-2-Aminophenyl-3-3-nitrophenyl-2-propen-1-one (BIA);
(2) 2-(5-(3-(trifluoromethyl)phenyl)isoxazol-3-yl)aniline (GM-90340);
(3) 2-(5-(3-(trifluoromethyl) phenyl) isoxazol-3-yl) aniline (GM-90339);
(4) (Z)-3-((E)-3-(2-aminophenyl)-3-oxoprop-1-and-1-yl)-N′-hydroxybenzimidamide (GM-90338);
(5) 3-(3-(2-aminophenyl)isoxazol-5-yl)benzonitrile (GM-90337);
(6) (E)-1-(2-amino-4,5-dimethoxyphenyl)-3-(3-bromo-5-hydroxyphenyeprop-2-en-1-one (GM-90321);
(7) (E)-1-(2-amino-4,5-dimethoxyphenyl)-3-(3-(trifluoromethyl)phenyeprop-2-en-1-one (GM-90320);
(8) (E)-3-(3-(2-amino-4,5-dimethoxyphenyl)-3-oxoprop-1-en-1-yl)benzonitrile (GM-90319);
(9) (E)-1-(2-aminophenyl)-3-(3-fluorophenyl)prop-2-en-1-one (GM-90318);
(10) (E)-1-(2-aminophenyl)-3-(3-chlorophenyl)prop-2-en-1-one (GM-90317);
(11) (E)-1-(2-aminophenyl)-3-(3,5-difluoro-4-hydroxyphenyl)prop-2-en-1-one (GM-90316);
(12) (E)-1-(2-aminophenyl)-3-(3-aminophenyl) prop-2-en-1-one hydrochloride (GM-90315);
(13) (E)-3-(3-(2-aminophenyl)-3-oxoprop-1-en-1-yl)-N-methyl benzamide (GM-90300);
(14) tert-butyl (E)-(3-(3-(2-aminophenyl)-3-oxoprop-1-en-1-yl)phenyl) carbamate (GM-90299);
(15) (E)-1-(2-amino-5-fluorophenyl)-3-(3-nitrophenyl)prop-2-en-1-one (GM-90298);
(16) (E)-1-(2-amino-4-methoxyphenyl)-3-(3-nitrophenyl)prop-2-en-1-one (GM-90297);
(17) (E)-3-(3-(2-aminophenyl)-3-oxoprop-1-en-1-yl)benzoic acid (GM-90296);
(18) (E)-1-(2-aminophenyl)-3-(3-ethoxyphenyl)prop-2-en-1-one (GM-90295);
(19) (E)-1-(2-aminophenyl)-3-(pyridin-3-yl)prop-2-en-1-one (GM-90285);
(20) (E)-1-(2-aminophenyl)-3-(m-tolyl)prop-2-en-1-one (GM-90284);
(21) (E)-1-(2-aminophenyl)-3-(3-(trifluoromethyl)phenyl)prop-2-en-1-one (GM-90283);
(22) (E)-3-(3-(2-aminophenyl)-3-oxoprop-1-en-1-yl)benzonitrile (GM-90282);
(23) (E)-1-(2-aminophenyl)-3-(3-bromo-5-hydroxyphenyl)prop-2-en-1-one (GM-90281);
(24) (E)-1-(2-amino-4,5-dimethoxyphenyl)-3-(3-methoxyphenyl)prop-2-en-1-one (GM-90256);
(25) (E)-1-(2-amino-4,5-dimethoxyphenyl)-3-(3-bromophenyl)prop-2-en-1-one (GM-90255);
(26) (E)-1-(2-amino-4,5-dimethoxyphenyl)-3-(3-nitrophenyl)prop-2-en-1-one (GM-90254);
(27) (E)-1-(2-aminophenyl)-3-(4-(tert-butyl)phenyl)prop-2-en-1-one (GM-90243);
(28) 1-(2-aminophenyl)-3-(2-ethoxy-5-nitrophenyl)-3-hydroxypropan-1-one (GM-90230);
(29) (E)-1-(2-aminophenyl)-3-(2-ethoxy-5-nitrophenyl)prop-2-en-1-one (GM-90229);
(30) (E)-1-(2-aminophenyl)-3-(pyridin-4-yl)prop-2-en-1-one (GM-90228);
(31) (E)-1-(2-aminophenyl)-3-(p-tolyl)prop-2-en-1-one (GM-90227);
(32) (E)-1-(2-aminophenyl)-3-(4-(trifluoromethyl)phenyl)prop-2-en-1-one (GM-90226);
(33) (E)-1-(2-aminophenyl)-3-(naphthalen-2-yl)prop-2-en-1-one (GM-90225);
(34) (E)-1-(2-aminophenyl)-3-(2-ethoxy-4-fluorophenyl)prop-2-en-1-one (GM-90224);
(35) (E)-1-(2-aminophenyl)-3-(2,4-difluorophenyl)prop-2-en-1-one (GM-90223);
(36) (E)-1-(2-aminophenyl)-3-(4-(trifluoromethoxy)phenyl)prop-2-en-1-one (GM-90222);
(37) (E)-1-(2-hydroxyphenyl)-3-(3-nitrophenyl)prop-2-en-1-one (GM-90135);
(38) (E)-1-(2-methoxyphenyl)-3-(3-nitrophenyl)prop-2-en-1-one (GM-90134);
(39) (E)-1-(2-aminophenyl)-3-(4-nitrophenyl)prop-2-en-1-one (GM-90133);
(40) (E)-1-(2-aminophenyl)-3-(3-bromophenyl)prop-2-en-1-one (GM-90132);
(41) (E)-1-(2-aminophenyl)-3-(3-methoxyphenyl)prop-2-en-1-one (GM-90131);
(42) (E)-1-(2-aminophenyl)-3-phenylprop-2-en-1-one (GM-90130);
(43) (E)-1-(2-aminophenyl)-3-(3-nitrophenyl)prop-2-en-1-one (GM-90129); and
(44) (E)-3-(3-nitrophenyl)-1-phenylprop-2-en-1-one (GM-90128);

In one particular embodiment of the present disclosure, the compound having the formula (1) is in the form of a racemate, an enantiomer, a diastereomer, or a mixture of diastereomers.

In one particular embodiment of the present disclosure, the formula (1) of the present disclosure can be used in the form of a pharmaceutically acceptable salt, and as a salt, an acid additive salt formed by a pharmaceutically acceptable free acid is useful. Acid-added salts are inorganic acids such as hydrochloric acid, nitric acid, phosphoric acid, sulfuric acid, hydrobromic acid, hydroiodic acid, nitrous acid or phosphorous acid. It is obtained from non-toxic organic acids such as alkanedioates, aromatic acids, aliphatic and aromatic sulfonic acids. Such pharmaceutically nontoxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, malate, butin-1,4-dioate, hexane-1,6-dioate, benzoate, chlorobenzoate, methyl benzoate, dinitro benzoate, hydroxybenzoate, methoxy benzoate, phthalates, terephthalates, benzene sulfonates, toluene sulfonates, chlorobenzene sulfonates, xylene sulfonates, phenylacetate, phenylpropionate, phenylbutyrate, citrate, lactate, β-hydroxybutyrate, glycolate, malate, tartrate, methane sulfonate, propane sulfonate, naphthalene-1-sulfonate, naphthalene-2-sulfonate or mandelate.

The acid addition salt according to the present disclosure is dissolved by conventional methods. For example, Formula 1 in an excess aqueous acid solution, wherein the salt is mixed with a water-miscible organic solvent such as methanol, ethanol, acetone or acetonitrile. It can be prepared by precipitating using it. The same amount of acid or alcohol in the formula 1 and water may be heated and then evaporated and dried or the precipitated salt may be prepared by suction filtration.

In addition, bases can be used to make pharmaceutically acceptable metal salts. Alkali metal or alkaline earth metal salt is obtained, for example, by dissolving the compound in excess alkali metal hydroxide or alkaline earth metal hydroxide solution, filtering the non-toxic compound salt, and evaporating and drying the filtrate. At this time, it is pharmaceutically suitable to prepare sodium, potassium or calcium salts as metal salts. In addition, the corresponding silver salt is obtained by reacting an alkali metal or alkaline earth metal salt with a suitable negative salt (eg, silver nitrate).

Additionally, Formula 1 of the present disclosure contains pharmaceutically acceptable salts in addition to adducts like hydrates and solvates essential for containing parent compounds or drugs.

The additive salts according to the present disclosure can be prepared by conventional methods, for example, the compound of formula 1 is dissolved in a water-miscible organic solvent such as acetone, methanol, ethanol, or acetonitrile, and an excess of organic acid is added. It can be prepared by adding an aqueous solution of inorganic acid and then precipitating or crystallizing. The solvent or excess acid can then be evaporated in this mixture and then dried to obtain an additional salt or the precipitated salt can be prepared by aspiration filtration.

In one aspect of the present disclosure, a composition for prevention, treatment, and improvement of a disease related to BI-1 (TMBIM6) comprising a compound of Formula 1, a pharmaceutically acceptable salt thereof, a hydrate thereof, or a solvate thereof as an active ingredient provides.

In one aspect of the present disclosure, a pharmaceutical composition for prevention, treatment, and improvement of mTORC2 related disease comprising a compound of Formula 1, a pharmaceutically acceptable salt thereof, a hydrate thereof, or a solvent thereof as an active ingredient provides.

In one aspect of the present disclosure, a pharmaceutical composition for prevention, treatment, and improvement of an AKT related disease comprising of a compound of Formula 1, a pharmaceutically acceptable salt thereof, a hydrate thereof, or a solvate thereof as an active ingredient provides.

In another embodiment of the present disclosure, the compound of Formula 1, a pharmaceutically acceptable salt thereof, a hydrate thereof, or a solvent thereof is administered in a therapeutically effective amount to an individual in need of treatment as an active ingredient. Alternatively, treatment methods are provided.

The disease may be a BI-1 related disease, an mTORC2 related disease, and an AKT related disease. The disease may be, but is not limited to, cancer, asthma or coronavirus infection.

When the compositions of the present disclosure are used as medicinal products, the pharmaceutical compositions containing the compounds of Formula 1 or pharmaceutically acceptable salts thereof as active ingredients may be formulated and administered in various oral or parenteral dosage forms at clinical administration. However, but is not limited to.

Formulations for oral administration include, for example, tablets, pills, light/soft capsules, liquids, suspensions, emulsifiers, syrups, granules, elixir agents, and the like, which are diluents (e.g. lactose, dextrose, sucrose, mannitol, sorbitol, cellulose and/or glycine), active agents (e.g., silica, talc, stearic acid and magnesium or calcium salts thereof and/ or polyethylene glycol). Tablets may also contain binders such as magnesium aluminum silicate, starch paste, gelatin, methylcellulose, sodium carboxymethylcellulose and/or polyvinylpyrrolidone, and in some cases, starch, agar, It may contain disintegrants or boiling mixtures such as alginic acid or sodium salts thereof and/or absorbents, colorants, flavoring agents, and sweeteners

The compound of formula 1 may be an antagonist of BI-1 (TMBIM6).

In addition, Formula 1 inhibits the activity of mTOR; or reduces phosphorylation of AKT or S6K; can be characterized by.

The present disclosure provides, in one embodiment, a dietary supplement composition comprising a compound of formula 1, a salt thereof, a hydrate thereof, or a solvent thereof.

The present disclosure provides, in one embodiment, a method of inhibiting BI-1, mTORC2 or AKT using a compound of formula 1, a salt thereof, a hydrate thereof, or a solvate thereof.

In one embodiment, the present disclosure provides a composition for inhibiting BI-1, mTORC2 or AKT comprising a compound of formula 1, a salt thereof, a hydrate thereof, or a solvate thereof, and a kit comprising the composition. Inhibition of BI-1, mTORC2 or AKT may be performed in vitro.

The compounds of formula 1 of the present disclosure, pharmaceutically acceptable salts thereof, hydrates thereof or solvates thereof inhibit calcium free by BI-1 and reduce the binding of BI-1 to mTORC2 thereby reducing mTORC2 activity, which reduces the activity of AKT;

Thus, the compounds of formula 1 of the present disclosure, the pharmaceutically acceptable salts thereof, hydrates thereof, and solvates thereof have the effect of preventing, treating, improving and alleviating symptoms of diseases or disorders related to BI-1, mTORC2, and AKT. For example, it has the effect of inhibiting the growth and metastasis of cancer, infectious diseases, asthma, penetration of coronavirus and exacerbation of infection, etc., and is not limited thereto.

### BRIEF DESCRIPTION OF THE DRAWINGS

FIGS. 1A-1E show mRNA expression profiling data of BI-1 (TMBIM6) for several tumor samples provided by NCBI's GEO (Gene Expression Omnibus). For example, FIGS. 1A-1E show Fibrosarcoma (GSE2719; Normal n=3; tumor n=7) shown in FIG. 1A, cervix (GSE63678; cervical normal n=5; Zhongyang n=5; Endometrial normal n=5; tumor n=7; vulvar normal n=7; tumor n=6) shown in FIG. 1B, breast (GSE31448; Normal n=31; Basal n=98; luminal A n=89; Tourism B n=49; ERBB2 n=25) shown in FIG. 1C, lung (GSE19804; Normal n=60; tumor n=60) shown in FIG. 1D, and prostate (GSE69223; Normal n=15; tumor n=15) shown in FIG. 1E. The center line of the box is the median; Box bounds are 25th and 75th percentages; whiskers represent minimum and maximum values.

FIG. 2 shows tissue microarray results comparing the expression level of BI-1 (TMBIM6) in the same cancer tissue as the sample of FIGS. 1A-1E. To validate the method, TMBIM6 (ie, BI-1) WT and knockout HT1080 cells were used as controls. The graph below of FIG. 2 quantifies the expression of BI-1 (TMBIM6). Fibrosarcoma (normal n=9; tumor n=8); cervix (normal n=20, tumor n=80); breast (normal n=6, tumor n=97); Lungs (normal n=15; tumor n=75); Prostate (normal n=32; tumor n=160). The scale bar is 100 μm. (black circle: positive antibody staining, white triangle: hematoxylin nuclear staining). Data were expressed as mean±SD. ****p<0.0001, two-tailed unpaired t-test.

FIGS. 3 to 5 are Kaplan-Meier curves showing a correlation between low and high expression of BI-1 (TMBIM6) and overall survival (OS) using GEPIA2 and OncoLnc using sources from TCGA and GTEx projects. In FIGS. 3 to 5, BRCA represents Breast Invasive Carcinoma, CESC represents Cervical Squamous Cell Carcinoma and Cervical Adenocarcinoma, SARC represents sarcoma, LUAD represents Lung adenocarcinoma, PAAD represents Pancreatic adenocarcinoma, ESCA represents Esophageal carcinoma, SKCM represents Skin melanoma, HNSC represents Head and neck squamous cell carcinoma, and LGG represents Sub brain glioma.

FIGS. 6A-6C show the preparation of BI-1 (TMBIM6) knockout cells using CRISPR/Cas9 genome editing techniques.

FIG. 7A shows proliferation of WT (WT) and BI-1 (TMBIM6) knockout (KO) HT1080 cells, HeLa cells and MEFs (n=3, independently experimented). FIG. 7B shows the proliferation of BI-1 knockout HT1080 and HeLa cells in which BI-1 has been rescued (n=3, independently experiment). Data were expressed in mean±SD, **p<0.01, ***p<0.001, ****p<0.0001. After the binary ANOVA, Bonferroni's post hoc test is performed.

FIGS. 8A and 8B are images and quantification of the results of cell migration (FIG. 8A) and cell invasion (FIG. 8B) experiments in BI-1 (TMBIM6) knockout cells and WT cells. The quantified data represent the percentage of WT cells normalized to knockout cells (migration experiment n=6 for migration, infiltration experiment n=5, each independent experiment). The scale bar represents 100 μm. Data are mean±SD. ****p<0.0001, member ANOVA followed by Tukey's post hoc test.

FIG. 9A shows subcutaneous injection of BI-1 (TMBIM6) WT (WT) and knockout (KO) HT1080 cells on the left and right sides of immunocompromised mice.

FIGS. 9B-9D show the volume, weight, and size of tumors obtained from BI-1 (TMBIM6) knockouts or WT HT1080 cells injected into the sides of 6-week-old nude mice (n=6 per group). Data were represented by mean±SD, *p<0.05; **p<0.01; ****p<0.0001, followed by a binary ANOVA, followed by Bonferroni's post hoc test in 9B and a two-tailed unpaired t-test in 9C.

FIG. 10 is the result of immunohistochemical staining of Ki67-positive proliferative cells. The right is a quantification of Ki-67 positive cells in Xenograft tumors derived from BI-1 (TMBIM6) knockouts and WT HT1080 cells. n=6 mice per group, scale bar represents 100 μm. Data average±SD. ****p<0.0001, two-tailed unpaired t-test.

FIGS. 11A-11C show the volume, weight and size of tumors derived from BI-1 (TMBIM6) knockouts and WT HeLa cells injected into the sides of nude mice (n=6 mice per group) at 6 weeks of age. Data were expressed as mean±SD. ***p<0.001; ****p<0.0001, Bon ferroni's post hoc test after binary ANOVA for tumor volume analysis, and Tukey's post hoc test after member ANOVA for tumor weight analysis.

FIG. 12 shows the results of immunohistochemical staining of Ki67-positive proliferative cells. The right is quantification of Ki-67 positive cells in xenograft tumors derived from BI-1 (TMBIM6) knockouts and WT HeLa cells. n=6 mice per group, scale bar represents 100 μm. The data averaged±SD. ****p<0.0001, followed by a binary ANOVA followed by Bonferroni's post hoc test.

FIGS. 13A-13D show the volume, weight, and size of tumors derived from HT1080 in TMBIM6-target SAMiRNA, control SAMiRNA or nude mice injected with saline solution (n=5 mice per group) through the tail vein. Data were expressed as mean±SD. *p<0.05, ***p<0.001, ****p<0.0001, Bonferroni's post hoc test (B) after binary ANOVA, member ANOVA followed by Tukey's post hoc test.

FIG. 14A is a result of qRT-PCR measurement of the mRNA level of TMBIM6 in a free tumor from HT1080 cells treated with TMBIM6 siRNA. n=5 mice per group. Data were expressed as mean±SD. ****p<0.0001, member ANOVA after Tukey's post hoc test.

FIG. 14B shows results of hematoxylin and eosin staining, immunohistochemical staining of tumors derived from HT1080 cells in nude mice injected with TMBIM6-target SAMiRNA, control SAMiRNA or saline solution through the tail vein. The picture was obtained from one of five xenograft mice with similar results.

FIG. 15 shows that in WT and BI-1 knockout HT1080 cells, expression and phosphorylation of 43 proteins were measured using the Proteome Profile Human Phospho-Kinase Array. The photograph showed one of two experiments with similar results. On the right is a quantification of the relative phosphorylation of the indicated protein with Image J.

FIG. 16 shows the results of analysis of pAKT, pTSC2 and pNDRG1 in BI-1 knockout and WT HT1080 cells with Western blot (left), and the right is normalized to total proteins of WT cells (n=5, independent experiment). Data were expressed as mean means±SD. ***p<0.001, ****p<0.0001, followed by a binary ANOVA followed by Bonferroni's post hoc test.

FIG. 17 shows the results of Western blotting, RT-PCR and gene quantitative analysis on TMBIM6 knockout cells that stably express or do not express TMBIM6-HA (n=3, independent experiment). Data were expressed as mean±SD. *p<0.05, **p<0.01, Bonferroni's post hoc test after binary ANOVA.

FIG. 18 shows the results of stimulating BI-1 (TMBIM6) knockout and WT HT1080 cells with insulin (100 ng/ml), IGF1 (100 ng/ml), or EGF (100 ng/ml) for 12 hours after serum depletion and Western blotting with the indicated antibody.

FIG. 19 shows the results of the gel filtration assay for extracts of BI-1 (TMBIM6) knockouts and WT MEFs. The one-point broken line represents a size marker.

FIG. 20 is the PLA result between the TMBIM6 knockout and the proteins shown in the WT HT1080 cell (fine and bright spot). For PLA, ribosomal protein S6 kinase beta-1 (S6K1) was used as a negative control. The scale bar represents 15 μm. The right side is a quantification of the fine and bright point (n=5, independently experimented). Data were expressed as mean±SD. *p<0.05, **p<0.01, followed by a binary ANOVA followed by Bonferroni's post hoc test.

FIG. 21 shows the anti-RPL19 immunoprecipitation (IP) and whole cell lysate of BI-1 knockout and WT HT1080 cells; WCL).

FIGS. 22A-22C show the results of the Western blot analysis of proteins labeled in HT1080, HeLa, and MEFs cells.

FIG. 23 is an immunofluorescence image of pAKT. On the right is the mean pAKT intensity of melted cells normalized to WT cells (n=10, independent experiment) (see priority application KR10-2021-0088876). Data were represented in flat±SD. ****p<0.0001, two-tailed unpaired t-test.

FIG. 24 is a Western blot analysis of proteins displayed in HeLa cells transfected with HA-TMBIM6.

FIG. 25 shows the results of Western blotting using indicated antibodies in BI-1 (TMBIM6) knockout MEFs cells transfected with TMBIM 6-HA with or without insulin (100 ng/ml) for 12 hours after serum starvation.

FIG. 26 shows the results of Western blotting of marked proteins in TMBIM6 T-Rex 293 cells treated with various concentrations of doxycycline for 24 hours.

FIG. 27 is the PLA result between the indicated proteins in the BI-1 knockout and WT HeLa cells (fine and bright points). Ribosomal protein S6 kinase was used as a negative control. The scale bar represents 15 μm. The bottom is a quantification of the fine and bright points (n=5, independent experiment). Data were expressed as mean±SD. **p<0.01, ****p<0.0001, followed by binary ANOVA followed by Bonferroni's post hoc test.

FIG. 28 shows the mRNA levels of marked proteins determined by qRT-PCR in BI-1 knockout and WT HT1080 cells (n=3, independent experiment). Data were expressed as mean±SD. After binary ANOVA, Bonferroni's post hoc test was used.

FIG. 29 shows Western blot analysis of anti-RICTOR IP and whole cell lysate (WCL) of BI-1 knockouts and WT MEFs.

FIG. 30 shows the results of polysomal profiling in BI-1 knockout and WT HT1080 cells with sucrose gradient fraction. (P) is the polysomal fraction, and (M) is the ribosomal fraction.

FIG. 31A is the result of performing a Western blot using an antibody marked with the fraction from FIG. 30.

FIG. 31B is the result of Western blot using an antibody showing a covector and a fraction from a BI-1 rescued knockout HT1080 cells.

FIG. 31C is the result of Western blot using antibodies indicated in purified poly (A) mRNA-bound ribosomes from HT1080 cells stably expressing BI-1 by oligo (dT) pull-down. Binding fractions and supernatants were indicated. FIGS. 31A, 31B, and 31C all showed one of two experiments with similar results.

FIG. 32 shows the results of staining cells for PDI (endoplasmic reticulum marker) and mTORC2 components by indirect immunofluorescence and quantifying co-localization (n=15 cells) (bottom) (see priority application KR10-2021-0088876). The scale bar quantifies the intensity of the dot in BI-1 knockout cells normalized to 10 and 5μm, and WT cells at the bottom. Data were expressed as mean±SD. ****p<0.0001, Bonferroni's post hoc test after binary ANOVA.

FIG. 33A shows the mRNA levels of glycolysis- and PPP-associated genes determined by qRT-PCR in BI-1 knockout and WT HT1080 cells. The quantification data represent the expression level of the genes in knockout cells compared to WT cells (n=3, independent experiment). Data are expressed as mean±SD. ****p<0.0001, Bonferroni's post hoc test after two-way ANOVA.

FIG. 33B and FIG. 33C show glucose consumption and lactic acid production in BI-1 knockout and WT HT1080 cells, respectively (n=3, independent experiment). Data were expressed in mean±SD. **p<0.01; ***p<0.001, Tukey's post hoc test after ANOVA.

FIG. 34 shows the mRNA levels of glycolysis- and PPP-related genes determined by qRT-PCR in BI-1 (TMBIM6) overexpressing HeLa cells. The quantification data represent covector (EV) cells normalized after normalized to the level of β-actin (n=2, independent experiment). Data were expressed as mean±SD. *p<0.05, **p<0.01, ***p<0.001, ****p<0.0001, Bonferroni's post hoc test after binary ANOVA.

FIG. 35 shows the results of the metabolic analysis in BI-1 knockout and WT HT1080 cells (n=2, independent experiment).

FIGS. 36A and 36B show mRNA levels of the GSH biosynthesis gene and de novo lipid biosynthesis gene determined by qRT-PCR in BI-1 knockout and WT HT1080 cells. Quantification data showed the level of expression of genes in knockout cells compared to WT cells (n=3 independent experiments). Data were expressed as mean±SD. *p<0.05, **p<0.01, ****p<0.0001, Bonferroni's post hoc test after binary ANOVA.

FIG. 37 is an immunofluorescence image of protein biosynthesis in BI-1 knockout and WT HT1080 cells. The quantification data on the right represent expression intensity compared to wild-type (WT) cells (n=3, independent experiment). Data were expressed as mean±SD. *p<0.05, two-tailed unpaired t-test. The scale bar is 15 μm.

FIG. 38A shows a list of genes related to glycosylation by microarray in BI-1 knockout and WT HT1080 cells (see priority application KR10-2021-0088876).

FIG. 38B shows glycosylated protein levels in BI-1 knockout and WT HT1080 cells (n=3, independent experiment). Data were expressed as mean±SD. *p<0.05, two-tailed unpaired t-test.

FIG. 39A is a result of the gel filtration assay of BI-1 (TMBIM6) knockout HT1080 cell lysates (lysates) that temporarily overexpress TMBIM6-HA.

FIG. 39B is the anti-RICTOR immunoprecipitation (IP) result of the pooled fraction of BI-1 (TMBIM6) knockout HT1080 cells temporarily overexpressing TMBIM6-HA. It was analyzed by Western Blotting.

FIG. 40A is a Western blot analysis of anti-HA IP and whole cell lysate (WCL) of HeLa cells overexpressing TMBIM 6-HA.

FIG. 40B is the PLA result between TMBIM6-HA and mTORC2 components (fine and bright spots) in HT1080 cells stably overexpressing BI-1 (TMBIM6). The graph below quantifies the fine and bright points (n=5, independent experiments), and the scale bar shows 15 μm. Data were expressed as mean±SD. ***p<0.001, ****p<0.0001, Bonferroni's post hoc test after two-way ANOVA.

FIG. 41A is the result of the GST pull-down analysis between GST-BI-1 and myc-RICTOR.

FIG. 41B shows the results of the GST pull-down analysis between HA-TMBIM6 and RPL19.

FIG. 42A is a Western blot analysis of the whole cell lysate (WCL) of HT1080 cells stably expressing BI-1 (TMBIM6) and transfected with scrambled, mTOR, RICTOR, or SIN1 siRNA and immunoprecipitated with anti-HA antibodies.

FIG. 42B shows the results of immunoprecipitate with anti-RICTOR antibodies of HT1080 cells transinfected with BI-1 (TMBIM6) and BI-1 mutation constructs and Western blot analysis of WCL.

FIG. 42C is a Western blot analysis of immunoprecipitation and input with anti-HA antibodies of HT1080 cells transfected with BI-1 (TMBIM6) and BI-1 mutation constructs.

FIG. 43A is a bioinformatic prediction result for the topology of BI-1 according to TMpred, TMHMM and BsYetJ. The box and number represent the membrane pass, domain, and amino acid, respectively.

FIG. 43B is the result of amino acid sequence alignment between BI-1 (TMBIM6) and BsYetJ based on the above description. The box and line each represent the same or alternative prediction sequence of A.

FIG. 44 shows immunofluorescence using cells overexpressing BI-1 (TMBIM6) tagged with N-terminal (HA-TMBIM6) and C-terminal (TMBIM6-HA) HA tags after permeabilization by digitonin or Triton X-100. The picture shows one of five experiments with similar results (see priority application KR10-2021-0088876).

FIGS. 45A and 45B show the results of confirming a protein interacting with BI-1. FIG. 45A shows a schematic diagram of the protocol for T4 phage display screening by plate method.

FIG. 45B shows amino acid sequence of identified protein

FIG. 46A is the PLA result between the indicated proteins (fine and bright spots) in HT1080 cells treated with BAPTA-AM (10 μM), BAPTA (10 μM), and EGTA-AM (10 μM). The scale bar represents 15 μm. The graph on the right quantifies the fine, bright points (n=3, independent experiment). Data were expressed as mean±SD. **p<0.01; ****p<0.0001, Tukey's post hoc test after member ANOVA.

FIG. 46B illustrates TMBIM6-GCaMP3 by a genetically encoded Ca2+ indicator (GCaMP3) directly fused to the C-terminus of BI-1 (TMBIM6).

FIG. 47A is the result of staining knockout cells with rescued knockout cells with expression of BI-1 (TMBIM6) and D213A (see priority application KR10-2021-0088876).

FIG. 47B illustrates the interaction of BI-1-leaking Ca2+ and with mTORC2 and ribosomal complexes.

FIG. 48 shows the immunofluorescence image (left) and fluorescence intensity (right) of TMBIM6-GCaMP3 and TMBIM6 D213A-GCaMP3 with and without 10 μM BAPTA-AM (n=5 independent experiment) (priority See application KR10-2021-0088876). The scale bar represents 15 μm. Data were expressed as mean±SD. **p<0.01, ***p<0.001, Bonferroni's post hoc test after binary ANOVA.

FIG. 49 shows PLA results between Rictor and mTOR or between Rictor and RPL19 in empty vector, BI-1 (TMBIM6) or D213A-transfected HT108 cells (fine and bright spots). (n=3 independent experiment). The scale bar represents 15 μm. Data were expressed as mean±SD. ****p<0.0001, Bonferroni's post hoc test after binary ANOVA.

FIG. 50 shows the findings of a Western blot analysis performed on cell lysates immunoprecipitated with anti-HA antibodies and specifically labeled antibodies.

FIG. 51A is a Western blotting and a quantification graph of AKT phosphorylation in BI-1 (TMBIM6) knockout HT1080 cells transfected with covectors, BI-1 (TMBIM6) WT and TMBIM6 D213A (n=3 independent experiment). Data were expressed as mea±SD. **p<0.01, ***p<0.001, Tukey's post hoc test after ANOVA.

FIG. 51B is an immunofluorescence image of AKT phosphorylation (see priority application KR10-2021-0088876) and quantification graph. (n=3 Independent experiment, sum of 9 images). The scale bar represents 15 μm. Data were expressed as mean±SD. ****p<0.0001, Tukey's post hoc test after member ANOVA.

FIG. 52 shows the results of the proliferation analysis of covector, BI-1 and TMBIM6 D213A-expressing HT1080 cells (n=3 independent experiment). Data were expressed as mean±SD. **p<0.01, ****p<0.0001, followed by a binary ANOVA followed by Bonferroni's post hoc test.

FIG. 53A shows the results of the quantification analysis of mRNA levels of covectors, BI-1 and TMBIM6 D213A-rescued BI-1 (TMBIM6) knockout HT1080 cells by qRT-PCR. The quantification data represent the expression level of the gene compared to the gene expression level of normalized WT HT10880 cells (horizontal line in the middle of the graph, n=3 independent experiment). Data were expressed as mea±SD. *p<0.05, **p<0.01, ***p<0.001, Bonferroni's post hoc test after binary ANOVA.

FIG. 53B and FIG. 53C show glucose consumption and lactic acid production in covector, BI-1 (TMBIM6) and TMBIM6 D213A-rescued BI-1 (TMBIM6) knockout HT1080 cells (n=3 independent experiment). Data were expressed as mean±SD. *p<0.05, **p<0.01, Tukey's post hoc test after member ANOVA.

FIG. 53D shows the results of metabolite analysis in covector, BI-1 (TMBIM6) and TMBIM6 D213A-rescued BI-1 (TMBIM6) knockout HT1080 cells. The quantification data represent the level of metabolites compared to the metabolites levels of covector-rescued BI-1 (TMBIM6) knockout HT1080 cells (n=2 independent experiment).

FIG. 54A is the result of Western blotting of BI-1-HA in covectors, BI-1 (TMBIM6), TMBIM6 D213A-rescued BI-1 (TMBIM6) knockout HT1080 cells.

FIG. 54B and FIG. 54C show the mRNA levels of the GSH biosynthesis gene (B) and de novo lipid biosynthesis gene (C) in covector, BI-1 (TMBIM6), TMBIM6 D213A-rescued BI-1 (TMBIM6) knockout HT1080 cells determined by qRT-PCR. The quantification data represent the expression level of the gene compared to the gene expression level of normalized WT HT1080 cells (horizontal line in the middle of the graph, n=3 independent experiment). Data were expressed as mean±SD. *p<0.05, **p<0.01, ***p<0.001, followed by Bonferroni's post hoc test.

FIG. 55 shows the results of metabolite analysis in covector, BI-1 (TMBIM6), TMBIM6 D213A-rescued BI-1 (TMBIM6) knockout HT1080 cells. The quantification data represent the level of metabolites compared to the metabolites levels of covector-rescued BI-1 (TMBIM6) knockout HT1080 cells (n=2 independent experiment).

FIG. 56 shows the results of polysomal profiling performed on covectors, BI-1 (TMBIM6), TMBIM6 D213A-rescued BI-1 (TMBIM6) knockout HT1080 cells by sucrose gradient fractionation.

FIG. 57 shows the proliferation of BI-1 (TMBIM6) WT HT1080 cells, BI-1 (TMBIM6) knockout HT1080, MCF7, MDA-MB-231 and SKBR3 cells treated with BIA (n=3 independent experiment). Data were expressed as mean±SD.

FIG. 58 is a result of gel filtration analysis of HT1080 cells treated with 1.0 μM BIA. The vertical line in the middle of the picture represents a size marker.

FIG. 59A shows Western blot assay results of anti-HA immunoprecipitation (IP) and whole cell lysate (WCL) of HT1080 cells temporarily overexpressing TMBIM6-HA.

FIG. 59B shows the results of Western blotting for p-AKT, AKT and actin after treating BIA on the indicated cell line.

FIG. 59C shows PLA results between TMBIM6-HA and mTORC2 components or between TMBIM6-HA and RPL19 in HT1080 cells stably overexpressing BIA-treated or untreated BIA (fine and bright dots). The graph below quantifies the fine and bright spots (n=5 independent experiment). The scale bar represents 20 μm. Data were expressed as mean±SD. **p<0.01, ****p<0.0001, followed by a binary ANOVA, followed by Bonferroni's post hoc test.

FIG. 60A and FIG. 60B show a real-time lapse image after BIA treatment on HT1080 cells stably overexpressing TMBIM6-GCaMP3 (FIG. 60A) and G-CEPIAer (FIG. 60B) (see priority application KR10-2021-0088876). On the right is the average green intensity of all cells normalized by BIA to untreated cells (n=5 independent experiment, total 20 cells for TMBIM6-GCaMP3; total 16 cells for G-CEPIAer). The scale bar represents 15 μm. Data were expressed as mean±SD.

FIG. 61A shows the mRNA level of BI-1 (TMBIM6) in various cancer cells. The mRNA level of each cell was normalized to the level of β-actin (n=3 independent experiment). Data were expressed as mea±SD. *p<0.05; **p<0.01; ***p<0.001, Tukey's post hoc test after member ANOVA.

FIG. 61B shows the results of measuring cell viability in cancer cells treated with BIA at the indicated concentrations for 3 days (n=3 independent experiment).

FIG. 62A shows the results of analyzing cell proliferation 1 day after treating cells and covectors stably expressing BI-1 at the indicated concentration of BIA (n=3 independent experiment). Data were expressed as mean±SD. ***p<0.001, ****p<0.0001, Bonferroni's post hoc test after binary ANOVA.

FIG. 62B is the result of PLA between RICTOR and the following proteins mTOR, RPL19 and RPS 16 in HT1080 cells treated with 10 μM BIA. The scale bar represents 20 μm. The graph on the right quantifies the fine and bright points (n=5 independent experiment). Data were expressed as mean±SD. **p<0.01, ***p<0.001, ****p<0.0001, followed by a binary ANOVA followed by Bonferroni's post hoc test.

FIG. 63A shows the Western blotting results of AKT phosphorylation.

FIG. 63B shows the results of cell viability analysis.

FIG. 64A is an image of cells for migration. The graph on the right shows the results of quantification analysis of migrated cells in BIA-treated BI-1 (TMBIM6) knockout HT1080 cells at the indicated concentration. Cell viability of BI-1 (TMBIM6) treated cells and migrated cells were normalized for control cells. (n=3 independent experiment).

FIG. 64B shows the results of the wound healing assay with HT1080 cells treated with 2 μM BIA. Image (A) and quantification graph (B) (n=3 independent experiment).

FIG. 65A is an image of cells migrated from HT1080, MCF7, MDA-MB-231, and SKBR3 cells treated with 2.0 μM BIA. The graph on the right quantifies migrated cells from BIA-treated cells normalized to control cells (n=3 independent experiments. Data were expressed in mean±SD. **p<0.01, ***p<0.001, two-tailed unpaired t-test simulation. The scale bar represents 15 μm.

FIG. 65B is an image of an invasive cell in HT1080 and MDA-MB-231 cells treated with 2.0 μM BIA. The graph on the right quantifies invading cells from BIA-treated cells normalized to control cells (n=3 independent experiments). Data were expressed as mean±SD. **p<0.01, ***p<0.001, two-tailed unpaired t-test. The scale bar represents 15 μM.

FIG. 66A is a DiI fluorescence image of the displayed cell line 7 days after 3D incubation. The right is a quantification of the fluorescence intensity of BIA-treated cells normalized to control cells (n=3 independent experiment). The scale bar represents 15 μm. Data were expressed as mean±SD. ***p<0.001, ****p<0.0001, two-tailed unpaired t-test.

FIG. 66B is an image of control and BIA-treated Zebrafish after injecting the indicated cell line into embryos. The circle marked with a dotted chain line and the circle marked with a dotted line represent the cell injection site and the mobile site, respectively. The image represents one of nine experiments that obtained similar results. The scale bar represents 100 μm.

FIG. 67A and FIG. 67B show the volume and weight of tumors derived from HT1080 cells injected into a vehicle or a nude mouse treated with 1 mg/kg BIA (vehicle; n=9, BIA; n=11 mice). Data were expressed as mean±SD. *p<0.05, ***p<0.001, p<0.0001, followed by a binary ANOVA followed by Bonferroni's post hoc test (A) or two-tailed unpaired t-test (B).

FIG. 67C and FIG. 67D show the volume and weight of tumors derived from MDA-MB-231 cells injected into a nude mouse treated with a vehicle or 1 mg/kg BIA. (n=5 mice for each group). Data were expressed as mean±SD. ***p<0.001, ****p<0.0001, followed by binary ANOVA, followed by Bonferroni's post hoc test (C) or two-tailed unpaired t-test (B).

FIG. 68 is a crystal violet staining image after treatment with 10 μM BIA and mTOR inhibitors on HT1080, PANC-1, Capan-1 and MIA PaCa-2. The graph on the right quantifies cell viability normalized to control cells (n=3 independent experiment).

FIG. 69 shows the results of the proximity ligation assay (PLA) between the indicated proteins in PANC-1 cells treated with BIA or mTOR inhibitors (fine and bright spots). The graph on the right quantifies the fine, bright points (n=5 independent experiment). The scale bar represents 20 μm. Data were expressed as mean±SD. **p<0.01, Bonferroni's post hoc test after binary ANOVA.

FIG. 70 shows the results of treating BIA or its analogue compound 10 μM with HT1080 fibrosarcoma cell line and DU145 prostate cancer cell line and measuring cell viability (n=3 independent experiments). Data were expressed as mean±SD. After one-way ANOVA, Tukey's post hoc test is conducted.

FIG. 71 shows the results of treating BIA and its analogue compounds 10 μM with HT1080 fibrosarcoma cell line to measure the degree of AKT serine phosphorylation (S473) using Western blotting. This is AKT signaling key to cancer cell growth, and BIA and its analogues tend to inhibit it. Only representative experimental results were presented. Data were expressed as mean±SD. After one-way ANOVA, Tukey's post hoc test is conducted.

FIG. 72 shows a Transwell insert (BD Biosciences, Franklin Lakes, NJ, USA) with 8.0 μm pores to see cell migration characteristics of cancer cells by treating each 10 μM of BIA and its analogs to HT1080 fibrosarcoma cell line. It was used and checked. Cells were treated with trypsin, and serum and 2×10⁴ cells were added to the upper chamber in DMEM, cells transported to the lower chamber via transwell for 12 hours were fixed and stained with crystal violet to perform migration analysis.

FIG. 73 shows the results of measuring cell invasion characteristics of cancer cells after treatment with BIA and its analogs in HT1080 fibrosarcoma cells. Cells were moved through the polyethylene terephthalate membrane (BD Biosciences) of the insert for 12 hours, and then fixed, crystal violet staining was performed to measure the degree of invasion.

FIG. 74A is a result of treating 10 μM BIA to HT1080 cells transfected to express TMBIM6-GCaMP3 and measuring the amount of calcium released through TMBIM6 over time.

FIG. 74B is a result of treating HT1080 cells with BIA 10 μM and observing the fluorescence intensity of G-CEPIAer designed to measure calcium concentration inside

ER.

FIG. 75 shows a calcium image coming out through BI-1 (TMBIM6) by observing a change in fluorescence when 10 μM of analogues including BIA were treated compared with cells in control (DMSO-treated) conditions in which fluorescence of TMBIM6-GCaMP3 is expressed.

FIG. 76A shows that OVA and LPS were treated with BI-1 (TMBIM6)WT WT(+/+) mice and knockout KO mice (−/−) to induce asthma.

FIG. 76B shows that a bronchoalveolar lavage fluid (BAL) sample (1 ml) of the asthma-inducing group of WT mice and BI-1 (TMBIM6) knockout mice was obtained from each mouse and red blood cells (Zap-Oglobin II; Beckman-Coulter) followed by a particle counter (Model Z1; Beckman-Coulter, Miami, FL, USA) to pool cell pellets to determine the total number of cells. Cells are placed in a slide, centrifuged (700 g×3 minutes), and stained with Diff-Quick (Baxter, Detroit, MI, USA) to show the result of detecting the number of inflammatory cells. FIG. 76C shows 10 uL of BAL samples of asthma-inducing groups of WT mice and BI-1 (TMBIM6) knockout mice mixed with 10 uL of 0.4% trypan blue and stained in trypan blue using a cell count meter (Countess Automated Cell Counter, Invitrogen, USA). The result is shown by measuring the total number of living cells excluding dead cells and the number of lymphocytes and neutrophils. Compared to WT, it was observed that the total number of these cells and the number of individual cells were suppressed under knockout conditions.

FIG. 77A is a methacholine test, in WT mice and BI-1 (TMBIM6) knockout mice, conscious mice after 3 days of the last challenge in a barometer volumetric chamber (All Medicus Co., Seoul, Korea), average of 3 minutes. The increase in aerosolized methacholine concentration (2.5˜50 mg/ml) was sprayed through the inlet of the main chamber for 3 minutes, and the value was determined during 3 minutes after a pause. Penh, (expiration time/relaxation time⁻¹)×(peak expiratory flow/peak intake flow) indicated the function of the maximum expiratory rate to the maximum intake box pressure signal according to the manufacturer's protocol. Penh is a prayer response to methacholine. The results were indicated by a percentage increase in the following Penh.

FIG. 77B shows interleukin (IL)-4 and IL-13 using ELISA kits (Endogen Inc., Woburn, Massachusetts, USA) in BALF of asthma inducing groups of WT mice and BI-1 (TMBIM6) knockout mice. Indicates the result of measuring the concentration. In the Standard setting, the lowest susceptibility value for the assay was 5 pg/ml.

FIG. 78A shows the expression level of IL-17 mRNA in the asthma-inducing group of WT mice and BI-1 (TMBIM6) knockout mice after isolating RNA from each tissue, making cDNA through reverse transcription reaction, and then using a primer of IL-17.

FIG. 78B and FIG. 78C are the results of fixing tissue to 4% formalin, injecting paraffin to make blocks, and then cutting them into 4 μm thick pieces to react to buffer, hematoxylin-eosin staining, and PAS staining.

FIGS. 79A to 79C show treating asthma-induced mice caused by asperagillus infection with BIA, IC87114, mixing 10 μL of BAL sample with 10 μL of 0.4% trypan blue, and then stained dead in trypan blue using a cytometer (Countess Automated Cell Counter, Invitrogen, USA). The results show observation of all inflammatory cells of living BAL, excluding cells.

FIG. 80A and FIG. 80B show mice that were infected with aspergillus with or without BIA, or IC87114, fixing the tissue to 4% formalin, injecting paraffin to make blocks, and then cutting into 4 μm thick to react to buffer and performing hematoxylin-eosin staining. The observation result (A) and the observation result (B) by PAS staining are shown.

FIG. 81 shows an aspergillus-infected asthmatic mouse model treated with IC87114 (PI3K inhibitor), dexamethasone, BIA or analog, mixing 10 μL of BAL sample with 10 μL of 0.4% trypan blue, followed by a cell count meter (Countess Automated Cell Counter, Invitrogen, USA). The result is quantified by counting the total number of cells of living BAL fluid, excluding dead cells stained in trypan blue.

FIG. 82 shows the results of MTT analysis confirming cell viability when treated with BIA or its analogues in a Vero E6 cell line infected with SARS-CoV2 virus. Data were expressed as mean±SD. Comparisons between groups were made using Dunnett's test with post-test (*, P<0.05 compared to SARS-CoV2-DMSO treatment group (DMSO-S), #, P<0.05 compared to control DMSO group (DMSO).

FIG. 83 is NMR data of GM-90223, a novel compound in the formula 1 of the present disclosure.

### DETAILED DESCRIPTION

The mechanistic target of rapamycin (mTOR) is known as a central regulator of signals regulating cell growth and metabolism that has been shown to be overexpressed in diabetic and cancer patients (Zoncu R. et al., ‘mTOR: from growth signal integration to cancer, diabetes and aging’ Nat Rev Mol Cell Biol (2011) Vol. 12(1), 21-35).

As an inhibitor of mTOR signaling, rapamycin was first identified and developed as a cancer treatment. However, it has been found that rapamycin only partially inhibits mTOR, so rapalog, sirolimus, and others have been developed as first-generation drugs of mTOR inhibitors. However, mTOR mutates well, and resistance to first-generation drugs has become a problem, and second and third-generation drugs have been developed to overcome this. However, due to the rapid mutation of mTOR, there are limitations in the development of drugs targeting mTOR. Therefore, it is necessary to develop new drugs that can overcome this.

BI-1 (Bax inhibitor-1) is also called TMBIM6 (transmembrane Bax inhibitor-1-containing motif family [6]). In this application, the names BI-1, Bax inhibitor-1, TMBIM6, and BI-1 (TMBIM6) are used interchangeably.

The inventors of the present application have shown that BI-1 activates mTORC2, which induces molecular and cellular signaling cascades through its properties of liberating calcium in the endoplasmic reticulum, and participating in the phosphorylation of AKT. Furthermore, inhibiting the calcium free phenomenon of BI-1 inhibits the binding of mTORC and BI-1 and at the same time inhibits the activation of mTORC2, which leads to the inhibition of phosphorylation of AKT and inhibits the growth of cancer cells. We identified compounds that antagonize the activity of BI-1.

The present inventors have identified mTORC2 activation regulated to the endoplasmic reticulum (ER), in particular BI-1, which is an important component for ER-related mTORC2 activation. Thus, the inventors found that inhibition of BI-1 inhibited mTORC2 activation and inhibited cancer growth. The inventors have completed the present disclosure by discovering compounds that inhibit BI-1.

The inventors have identified mTORC2 activation as the endoplasmic reticulum (ER) stress regulator. Specifically, BI-1 is essential in upregulating ER-associated mTORC2 activation in cancer cells. In particular, based on the calcium free nature in ER, mTORC2 and ribosomes were confirmed to be recruited, which led to the activation of mTORC2.

In addition, the inventors found that ER-related mTORC2 activation by BI-1 increases the expression of lipid synthesis genes that induce glycolysis, pentose phosphate pathways, and cancer progression. Accordingly, the inventors found that inhibiting the BI-1 (TMBIM6) gene inhibited mTORC2 activation and inhibited cancer growth.

The inventors discovered a new inhibitor of BI-1, 2E-1-2-aminophenyl-3-3-nitrophenyl-2-propen-1-one, which was named BIA. In fact, as a result of treating BIA on various cancer cells, it was found that BIA inhibits BI-1, reduces binding to mTORC2, and finally reduces mTORC1 and mTORC2 activity. In addition, BIA treatment on cancer-induced zebrafish and nude mice showed that the size and weight of cancer decreased. In addition, 43 analogs of the compound (BIA) presented below also inhibited cancer growth, cell migration and invasion, and phosphorylation of AKT in the cell model.

Thus, the BIA and 43 BI-1 antagonists of analogs of BIA were applied to prevent and treat BI-1 or mTORC, preferably mTORC2 related diseases.

BI-1 has been associated with liver diseases such as liver ischemic reperfusion injury, chronic hepatitis, and carbon tetrachloride-induced liver damage, and BI-1 deficiency has been reported to promote liver regeneration. In addition, BI-1 is associated with cancers such as tumorigenesis, prostate cancer, pulmonary adenocarcinoma, breast cancer, nasopharyngeal carcinoma, acute myeloid leukemia, autoimmune diseases, neurological diseases, and insulin resistance. (Li B. et al., The characteristics of Bax inhibitor-1 and its related diseases, Current Molecular Medicine 2014, 14, 603-615).

mTORC2 regulates cell metabolism and cell survival by activating AKT, a viable kinase. mTORC2 is also involved in the regulation of autophagy. m TORC2 plays an important role in metabolic regulation and has been associated with many related diseases. It is associated with metabolic diseases such as type 2 diabetes, for example. In addition, it has been reported that mTORC2 is overactivated in several types of cancer. mTORC2-mediated lipogenesis is known to promote hepatocellular carcinoma. The mTORC2 pathway plays an important role in the development of lung fibrosis (Chang W et al. “A critical role for the mTORC2 pathway in lung fibrosis”. (2014) PLOS ONE. 9 (8): e106155). Chronic mTORC2 activity is known to play an important role in systemic lupus erythematosus by impairing the function of ribosomes. In addition, mTORC2 has been proposed as a treatment for allergic diseases as it is involved in the polarization of Th9 cells and the regulation of allergic airway inflammation (Chen H, et al., “mTORC2 controls Th9 polarization and allergic airway inflammation”, Allergy (2017) 72(10):1510-1520). mTORC1 and mTORC2 signaling are (Kuss-Duerkop SK et al., “Influenza virus differentially activates mTORC1 and mTORC2 signaling to maximize late stage replication.” PLoS Pathog. 2017 Sep. 27; 13(9): e1006635).

AKT (also known as protein kinase B (PKB)) is involved in the regulation of cell proliferation, survival, and metabolism. Alterations with AKT function lead to many diseases, such as cancer, diabetes, and cardiovascular disease (Hers I. et al., Akt signaling in health and disease, Cellular Signaling (2011) Volume 23, Issue 10, 1515-1527).

The activation of AKT also plays an important role in promoting inflammation. For example, it also plays an important role in refractory asthma. The present disclosure suggests that the expression of BI-1 was inhibited under asthmatic conditions and that asthma symptoms and inflammatory cytokine increase were largely inhibited in severe asthma models in BI-1 knockout mice, and type II cytokines IL-4, IL-5 and IL-13 were inhibited under knockout conditions. When the BIA and its analogues were applied, asthma and related inflammatory cytokines were suppressed. In addition, the activity of AKT is an important signaling for the penetration of coronaviruses such as Covid-19 as it is involved in the process of Covid-19 binding to ACE2, an intracellular infection pathway, and endocytosis (Reis CR t al, Crosstalk between Akt/GSK3 β signaling and dynamin-1 regulates clathrin-mediated endocytosis, EMBO J. 2015 Aug. 13; 34(16): 2132-46. doi: 10.15252/embj.201591518.). The present disclosure shows that the administration of BIA, a BI-1 antagonist, and analogs (43 compounds) of BIA can prevent coronavirus infection.

As one embodiment of the present disclosure, there is provided a compound represented by the following formula (1) or a pharmaceutically acceptable salt thereof, a hydrate thereof and a solvate thereof:

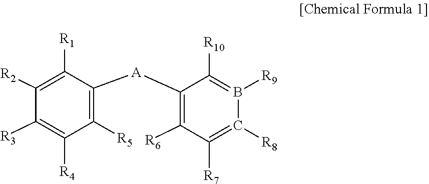

here A is the following formula 1-1, 1-2, or 1-3,

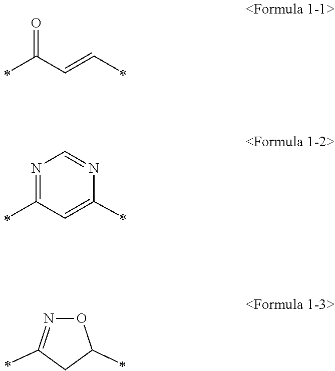

(* of formula 1-1 to 1-3 are connected to the phenyl group of formula 1)
   B and C are C, or N, respectively,
   the R₁ to R₁₀ are respectively H; NH₂; NO₂; OH; OR (R is a linear, branched alkyl, alkenyl, or alkynyl of C₁ to C₁₀); halogen atoms; CN; Alkyl halides of C₁ to C₃; a linear, branched alkyl, alhynyl, or alkynyl group of C₁ to C₁₀; —NH—C(O)—ORa (Ra is a linear, branched alkyl, alkenyl, or alkynyl of C₁ to C₁₀ IM); —C(O)—NH—RA (Ra is a linear, branched alkyl, alkenyl, or alkynyl of C₁ to C₁₀); —NH₂HCl; —C(O)OH; and substituents having a jade core; One or more selected from the group consisting of.
   if B or C is N, R₈ and R₉ are hydrogen,
   when B or C is C, R₈ and R₉ can be connected to form a phenyl ring,

The substituent having the jade core is represented by the following formula 1-4.

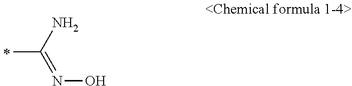

(* of formulas 1-4 is where substituents R₁ to R₁₀ of Formula 1 are substituted)

In one specific embodiment of the present disclosure, the compound of Formula 1 may be C in phases B and C of Formula 1, and A may be Formula 1-1, Formula 1-2 or Formula 1-3.

In one specific embodiment of the present disclosure, the compound of formula 1 comprises B and C of formula 1 are C, respectively, A is formula 1-1, and substituents R₈ and R₉ are combined to form a phenyl group. It may have been formed.

In one specific embodiment of the present disclosure, the compound of Formula 1 may be B or C of Formula 1 is N, and A may be Formula 1-1.

In one embodiment of the present disclosure, the compound of formula 1 may be selected from the following group:

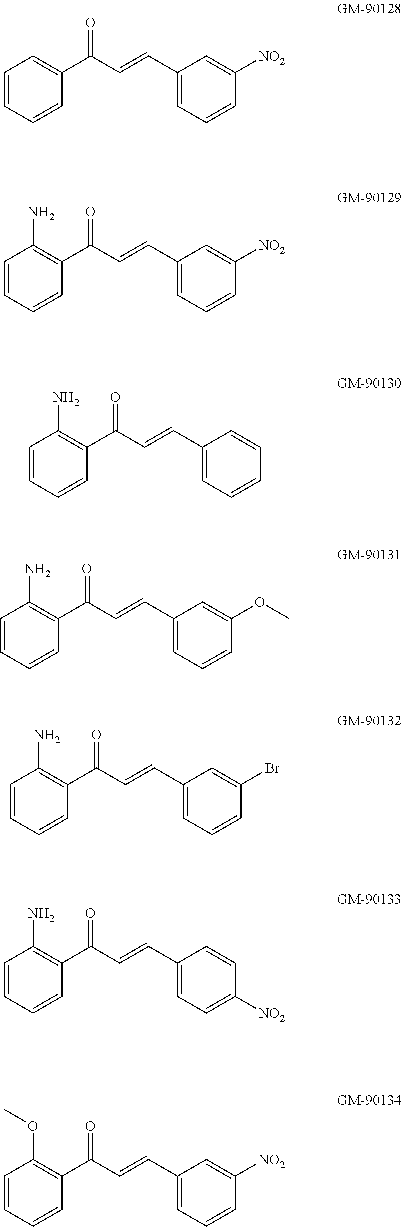

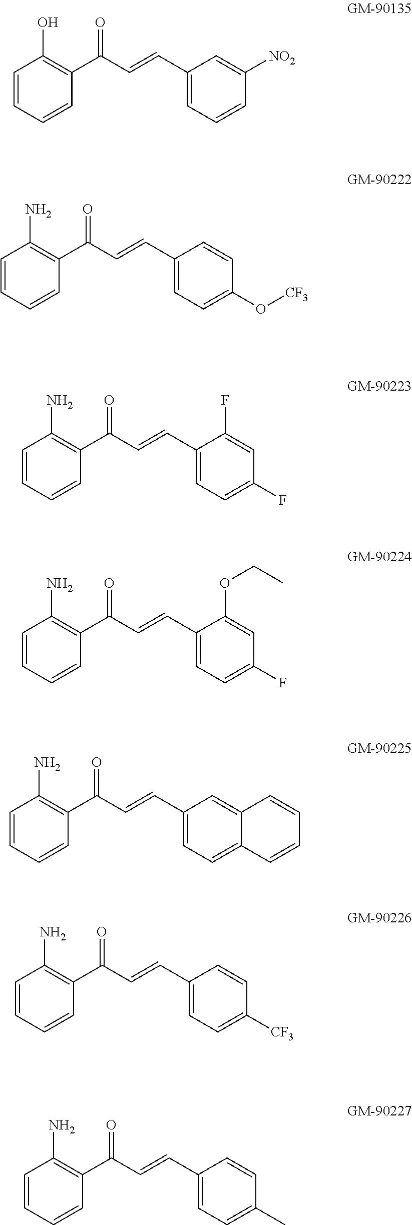

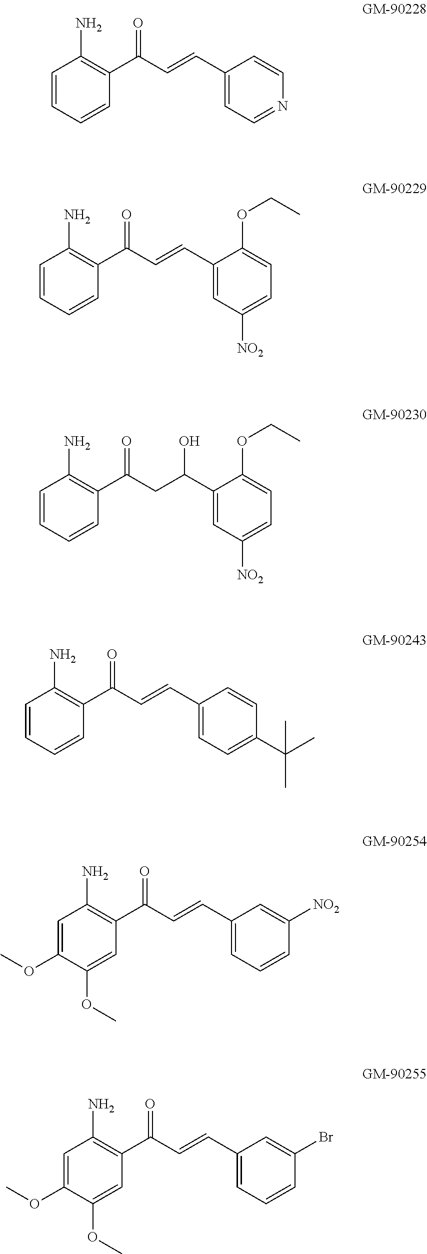

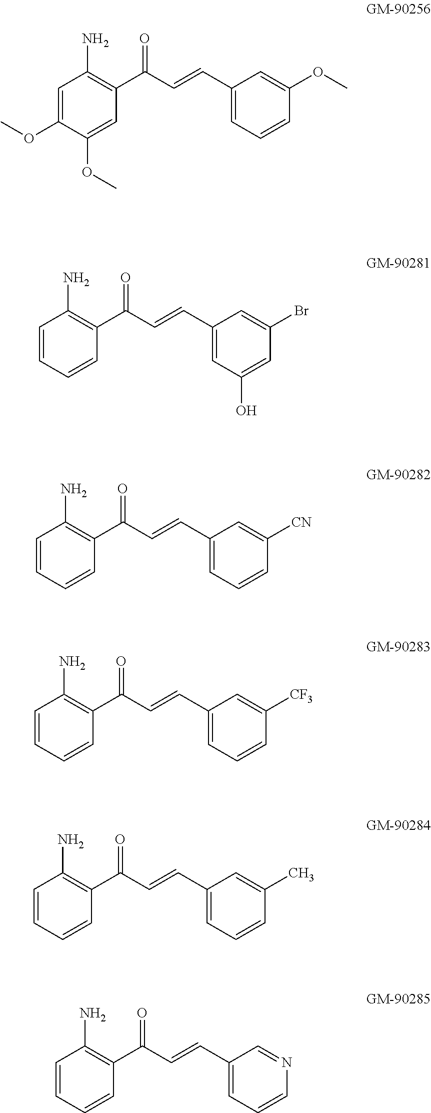

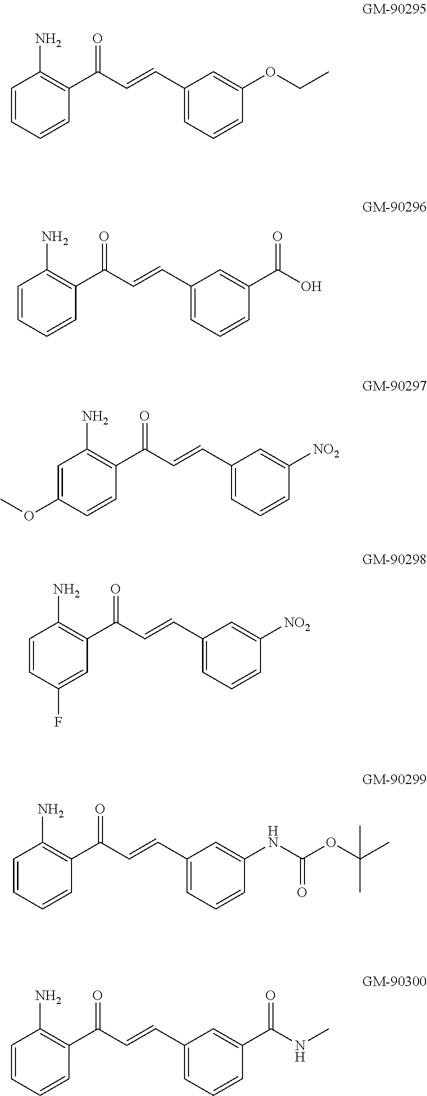

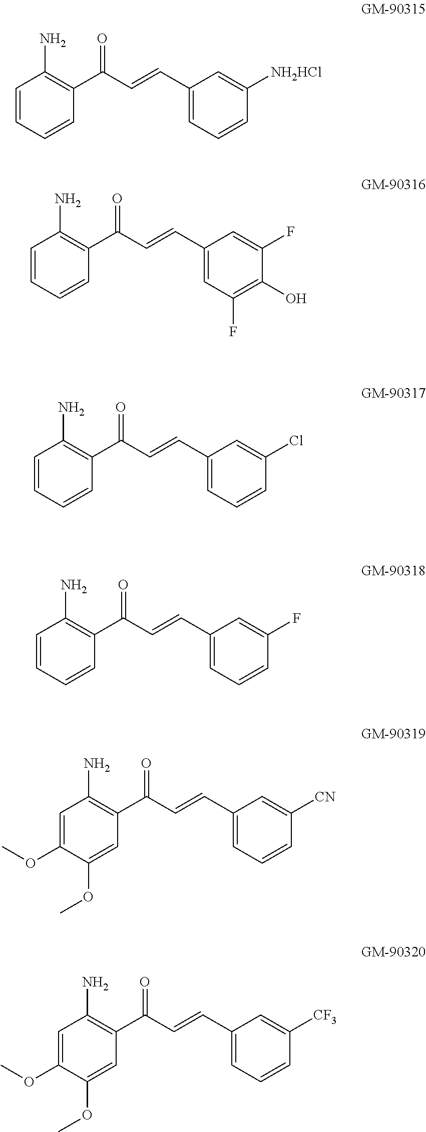

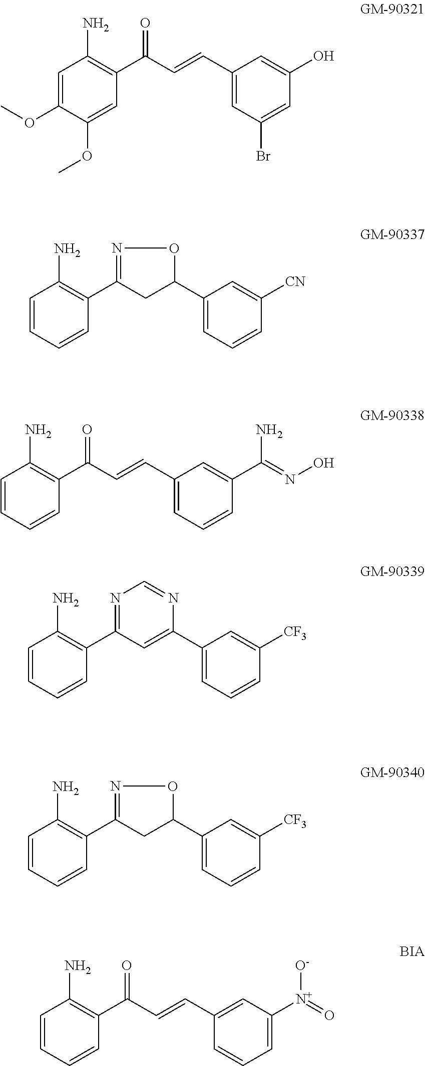 [SMILES: COc1cc(N)c(C(=O)/C=C/c2cc(O)cc(Br)c2)cc1OC ; N#Cc1cccc(C2CC(c3ccccc3N)=NO2)c1 ; N/C(=N\O)c1cccc(/C=C/C(=O)c2ccccc2N)c1 ; Nc1ccccc1-c1cc(-c2cccc(C(F)(F)F)c2)ncn1 ; Nc1ccccc1C1=NOC(c2cccc(C(F)(F)F)c2)C1 ; Nc1ccccc1C(=O)/C=C/c1cccc([N+](=O)[O-])c1]

The compound of formula 1 may be an inhibitor (antagonist) of BI-1.

The compound of formula 1 inhibits calcium release of BI-1 or the like, inhibits the activity of mTOR; or reduces phosphorylation of AKT or S6K; Can be characterized by.

The present disclosure provides as one embodiment a pharmaceutical composition comprising a compound of formula 1 or a pharmaceutically acceptable salt thereof, a hydrate thereof and a solvate thereof.

The present disclosure provides as one embodiment a pharmaceutical composition for preventing or treating a BI-1 related disease comprising a compound of formula 1 or a pharmaceutically acceptable salt thereof, a hydrate thereof, and a solvent thereof.

The present disclosure provides as one embodiment a pharmaceutical composition for preventing or treating a disease controlled by antagonism of BI-1, comprising a compound of formula 1 or a pharmaceutically acceptable salt thereof, a hydrate thereof and a solvate thereof.

Diseases controlled by the BI-1 include, for example, liver diseases such as, liver ischemia reperfusion injury, chronic hepatitis, carbon tetrachloride-induced liver damage, cancers such as pulmonary adenocarcinoma, breast cancer, nasopharyngeal carcinoma, and acute myeloid leukemia, autoimmune diseases, neurological diseases, insulin resistance, asthma, COVID infection, etc. This is not limited to this.

The present disclosure, in one embodiment, the compound of formula 1 or the pharmaceutically acceptable salts thereof, hydrates thereof and solvates thereof can be used in pharmaceutical compositions to promote the regeneration of the liver and the regeneration of other organs.

The present disclosure provides a pharmaceutical composition for preventing or treating mTORC2 related diseases comprising a compound of formula 1 or a pharmaceutically acceptable salt thereof, a hydrate thereof, and a solvent thereof, as one embodiment.

mTORC2-related diseases include metabolic diseases such as type 2 diabetes, cancer, respiratory diseases including lung fibrosis, asthma and COPD, viral infections, and systemic lupus erythematosus, but are not limited thereto.

The present disclosure provides as one embodiment a pharmaceutical composition for preventing or treating AKT related diseases comprising a compound of formula 1 or a pharmaceutically acceptable salt thereof, a hydrate thereof, and a solvate thereof.

AKT-related diseases include, cancer, diabetes, cardiovascular disease, inflammatory disease, respiratory disease including asthma and COPD Viral infections such as coronavirus infection, but are not limited thereto.

The cancers include lung cancer, lung adenocarcinoma, pancreatic cancer, colorectal cancer, myeloid leukemia, thyroid cancer, myelotype dysmorphic syndrome (MDS), bladder carcinoma, epidermal carcinoma, melanoma, breast cancer, prostate cancer, head and neck cancer, uterine cancer, ovarian cancer, brain cancer, stomach cancer, laryngeal cancer, esophageal cancer, bladder cancer, oral cancer, nasopharyngeal cancer, cancer of mesenchymal origin, fibrosarcoma, teratoma carcinoma, neuroblastoma, kidney carcinoma, liver cancer, non-Hodgkin lymph, multiple myeloma, and thyroid undifferentiated carcinoma, most preferably fibrosarcoma or breast cancer.

Asthma may include all asthma that has been favored to date, such as steroid-resistant asthma, including general allergic asthma.

The coronavirus infectious disease includes mutants by country by region, including COVID19, and may include all similar coronavirus infections such as MERS.

The pharmaceutical compositions of the present disclosure may further comprise suitable carriers, excipients and diluents commonly used in the preparation of pharmaceutical compositions. In addition, according to conventional methods, oral formulations such as acids, granules, tablets, capsules, suspensions, emulsions, syrups, aerosols, etc., can be formulated and used in the form of external agents, suppositories and sterile injection solutions. Suitable formulations are preferably those disclosed in the literature (Remington's Pharmaceutical Science, most recently, Mack Publishing Company, Easton PA). Carriers, excipients, and diluents that may be included include lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate and mineral oil. When the composition is formulated, it is prepared using diluents or excipients such as fillers, extenders, binders, wetting agents, disintegrants, and surfactants commonly used. Solids for oral administration include tablets, pills, acids, granules, capsules, and the like, which solids are included in the composition of at least one excipient such as starch, calcium carbonate(calcium carbonate), sucrose, lactose, gelatin, etc. are mixed.

In addition to simple excipients, lubricants such as magnesium stearate and talc are also used. Liquid preparations for oral use include suspensions, anti-solution agents, emulsions, syrups, etc., and in addition to water, liquid paraffin, which are commonly used as simple diluents, various excipients, such as wetting agents, sweeteners, air fresheners, preservatives, and the like. Preparations for parenteral administration include sterile aqueous solutions, non-aqueous solvents, suspensions, emulsions, lyophilized preparations, and suppositories. Non-aqueous solvents, and suspension agents may include propylene glycol, polyethylene glycol, vegetable oils such as olive oil, injectable esters such as ethyl oleate, and the like. As a substrate for suppositories, witepsol, macrogol, tween 61, cacao oil, laurin oil, glycerogelatin, and the like may be used.

As used herein, the term “administration” means to provide an individual with a composition of the present disclosure in any suitable manner.

Preferred dosages of the pharmaceutical compositions of the present disclosure vary depending on the condition and weight of the individual, the degree of disease, the form of the drug, the route of administration and the duration, but may be appropriately selected by those skilled in the art. For the desired effect, the pharmaceutical composition of the present disclosure may be administered in an amount of 0.1 mg/kg to 10 mg/kg per day, most preferably in an amount of 1 mg/kg, once or several times a day. It can be divided into doses.

The pharmaceutical compositions of the present disclosure can be administered to individuals by various routes. All modes of administration can be expected, for example, by oral, rectal or intravenous, intramuscular, subcutaneous, intrauterine dural or cerebrovascular injection.

The pharmaceutical compositions of the present disclosure can be used alone for the prevention and treatment of cancer, or in combination with methods using surgery, radiotherapy, hormone therapy , chemotherapy and biological response modulators.

The present disclosure provides a functional food composition for the prevention and treatment of cancer, treatment and prevention of asthma, the prevention, improvement, symptom relief or treatment of coronavirus infection comprising the compound represented by formula 1.

The compound of formula 1 may be an inhibitor of BI-1.

The compound of formula 1 inhibits the activity of mTOR; or reduces phosphorylation of AKT or S6K; Can be characterized by.

The cancers include lung cancer, lung adenocarcinoma, pancreatic cancer, colorectal cancer, myeloid leukemia, thyroid cancer, myelotype dysmorphic syndrome (MDS), bladder carcinoma, epidermal carcinoma, melanoma, breast cancer, prostate cancer, head and neck cancer, uterine cancer, Ovarian cancer, brain cancer, stomach cancer, laryngeal cancer, esophageal cancer, bladder cancer, oral cancer, nasopharyngeal cancer, cancer of mesenchymal origin, fibrosarcoma, teratoma carcinoma, neuroblastoma, kidney carcinoma, liver cancer, non-Hodgkin lymphoma, multiple myeloma, and thyroid undifferentiated cancer, which may be any one or more selected from the group, most preferably fibrosarcoma or breast cancer.

The asthma may include all asthma that has been favored to date, such as steroid-resistant asthma, including general allergic asthma .

The coronavirus infectious disease includes mutants by country by region, including COVID19, and may include all similar coronavirus infections such as MERS.

The health food composition may be used with other foods or food ingredients and may be appropriately used according to conventional methods. The mixed amount of the active ingredient can be suitably determined according to the purpose of use (for example, health or treatment). In general, in the manufacture of food or beverages, the compositions of the present disclosure are added to the raw material in an amount of 15% by weight, preferably 10%. However, in the case of long-term ingestion for health and hygiene or health control, it may be below the above range, and since there is no problem in terms of safety, the active ingredient is also in an amount greater than the above Can be used.

There are no special restrictions on the type of food. Examples of foods to which the above substances can be added include meat, sausage, bread, chocolate, candy, snacks, confectionery, pizza, ramen, other noodles, chewing gum, dairy products including ice cream , various Soups, beverages, teas, drinks, alcoholic beverages and vitamin complexes, all include health foods in the usual sense.

The health drink composition of the present disclosure may include various flavors or natural carbohydrates as additional ingredients such as conventional beverages. The above-mentioned natural carbohydrates can be used as monosaccharides such as glucose and fructose, disaccharides such as maltose and sucrose, natural sweeteners such as dextrin and cyclodextrin, synthetic sweeteners such as saccharin and aspartame , etc. There is. The ratio of the natural carbohydrates is generally about 0.01 to 10 g, preferably from about 0.01 to 0.1 g per 100 ml of the composition of the present disclosure.

In addition to the above, the compositions of the present disclosure include various nutritional agents, vitamins, electrolytes, flavor agents, colorants, pectic acids and salts thereof, alginic acid and salts thereof, organic acids, protective colloidal thickeners, pH adjusters, stabilizers, preservatives, glycerin, alcohol, carbonated agents used in carbonated drinks, and the like. In addition, the compositions of the present disclosure may include pulp for the production of natural fruit juice, fruit juice beverages and vegetable beverages. These components can be used independently or in combination. The ratio of such additives is not very important, but it is generally selected in the range of 0.01 to 0.1 parts by weight per 100 parts of the composition of the present disclosure.

Hereinafter, preferred embodiments will be presented to facilitate the understanding of the present disclosure. However, the following embodiments are only applied to more easily understand the present disclosure, and the contents of the present disclosure are not limited by the embodiments.

### Experimental Method

The experimental method performed in the following embodiments is as follows.

### Cell Culture

HT1080 MDA-MB-231, SKBR3 and MCF7 cell lines were cultured in a 37° C., 5% CO₂ incubator in a medium with 10% Fetal Bovine Serum, 1% penicillin and streptomycin (100 U) added to DMEM.

### Gel Filtration Analysis

HT1080 cells knocked out of BI-1 (using 8 10 cm cell culture dishes for each sample) were temporarily transformed using HA-BI-1 (TMBIM6) plasmids. HT1080 cells knocked out of BI-1 were treated with 10 μM of 2E-1-2-Aminophenyl-3-3-nitrophenyl-2-propen-1-one (BIA). After 24 hours, CHAPS containing protease inhibitor cocktail and phosphatase inhibitor cocktail Cells were lysed in 1.0 ml of buffer (pH 7.4, 150 mM NaCl, 1 mM EDTA and 25 mM HEPES at 0.3% CHAPS). The cell lysate was filtered with a 0.45 μm syringe filter. Using CHAPS buffer, the total protein concentration was adjusted to 5 mg/ml and 500 μl of lysate was placed in the Superdex 200 Increase 10/300 GL column (GE Lifesciences catalog number 28-9909-44), and AKTA-FPLC (GE Lifesciences Cat. No.18-1900-26). It was fractionated to 600 μ, at an elution rate of 0.3 ml/min. 50 μL of each fraction was electrophoresis with SDS-PAGE and then detected with various antibodies. The molecular weight resolution of the column was estimated using a gel filtration calibration kit (GE Lifesciences, 28-4038-42).

### Immunoblotting

After cell recovery, cell lytic buffer (10 mM Tris-Cl (pH 7.4), 5 mM EDTA, 130 mM NaCl, 1% Triton X-100) and protease inhibitor mix protease inhibitor cocktail (protease inhibitor cocktail and phosphatase inhibitor cocktail) were added and left at 4° C. for 30 minutes. After breaking the cells with ultrasound, they were centrifuged at 13,000× g at 4° C. for 30 minutes, and then the supernatant was taken, and the concentration was quantified using a protein quantification kit (Bio-Rad Laboratories, Hercules, CA, USA). 20 ug of the obtained protein was electrophoresis with polyacrylamide gel electrophoresis (SDS-PAGE) followed by electrotransfer to PVDF membrane (Bio-Rad). The membrane was blocked at room temperature for 1 hour with a TBS-T solution (20 mM Tris (pH 7.5), 137 mM NaCl, 0.05% Triton X-100) containing 5% skim milk. Then, after switching to a solution containing the primary antibody, it was reacted at 4° C. The membrane after the reaction was washed three times with TBS-T solution, reacted with a secondary antibody, and then luminesced using an ECL kit and autoradiography.

### Experiments on the Invasion of Tumor Cells

Cell invasion experiments were performed according to the method of the Invasion Assay Kit (corning, 3458). Put a cell culture insert into the 24-well cell culture plate, DMEM medium with 10% FBS added outside the insert , and no FBS added inside the insert. 2.0 μM BIA was added to HT1080 and MDA-MB-231 and incubated at 37° C. for 12˜24 hours. Removed the medium in the 24-well culture plate and the medium inside the insert Then, a cotton swab was used to remove the remaining cells in the insert. After fixing the insert in 4% formaldehyde solution, stain the cells that penetrate the bottom of the insert with crystal violet solution, wash it with tertiary distilled water, and then at room temperature Dried and dried inserts were observed under a microscope.

### Three-Dimensional Cell Culture

Three-dimensional cell culture was performed according to the method of Cellrix 3D Culture System (Medifab). Cells were labeled with 2 g/mL of DiI in vitro and then suspended at 1×106 cells/mL in Cellrix Bio-Gel. After removing the casting mold from the casting gel, carefully apply the Cellrix Bio-Gel with the cells suspended. It was busy. Bio-Gel, which had been dispensed in ice for 15 minutes, was gelated. The gelled Cellrix Bio-Gel 3D culture was gently pushed out with sterile tweezers and placed in a 96-well plate containing the drug. The medication was changed every three days. A week later, cell culture was observed using fluorescence microscopy (Ni knockout n Eclipse C1).

### Treatment of BIA in Zebrafish Tumor Model

Zebrafish fertilized eggs were incubated in Danieau solution at 28° C. and raised under standard laboratory conditions. At 48 hours after fertilization, zebrafish embryos were anesthetized with 0.04 mg/ml of tricaine (MS-222, Sigma). The anesthetized embryos were transferred to agarose gel for microinjection. Tumor cells were labeled with 2 g/mL of DiI in vitro before injection. About 100-500 tumor cells were resuspended in serum-free DMEM and 5 nL of tumor cell solution was injected into the perivitelline cavity of each embryo using a microinjector. A non-filamentous silicate glass capillary needle was connected to the microinjection. The injected embryos were immediately transferred to water maintained at 28° C. Using multi-blade fluorescence microscopy (Ni knockout n Eclipse C1), only fluorescent embryos were screened and randomly divided into control and experimental groups. The control group added 0.01% DMSO and the experimental group added 2 uM BIA and monitored tumor growth and invasion.

### Treatment of BIA in Nude Mice

Mice purchased 20-25 g of BALBc/Nude and conducted experiments after 1 week of preliminary breeding to adapt to the laboratory environment. This experiment was approved by the Animal Experiment Ethics Committee of Chonbuk National University (CBNU 201 5-064, CBNU 2016-56) and was carried out according to standard work instructions, and the conditions in the breeding room were kept constant temperature and relative humidity, a 12-hour contrast cycle, and water and feed were freely consumed. Experimental animals are transplanted subcutaneously with 5×10⁶ cells per mouse using HT1080 cell line and MDA-MB-231 cell line, and after 7-10 days, mice forming tumor sizes of 100 mm³ are randomly divided into control and experimental groups. The control group is injected with saline (including 10% DMSO) and the experimental group is injected with 1 mg/kg (10% DMSO) into the abdomen. The drug is given 5 days a week, and repeated for 4 weeks. After 28 days, mice were euthanized and tumors were extracted to specify weight and size. The tumor size was measured with a caliper and calculated using the following formula:

Tumour volume (mm³)={(shortest diameter)×2×(longest diameter)}/2

Reverse Transcription-Quantitative Real-Time (qRT-) PCR

Total RNA was extracted from cancer cells using TRIzol reagent (Invitrogen), and 3 μg was used to generate cDNA with the SuperScript III First-Strand Synthesis Kit (Invitrogen) according to the manufacturer's protocol. The sequences of the primer pairs used in this study are as follows.

TABLE 1

| Name | Forward (5′-3′) | Reverse (5′-3′) |
|---|---|---|
| TMBIM6 | AGCAGCACCTGAAGAAGGTC | TCAATATCAGGGAGCCCAAG |
| | (SEQ ID NO: 1) | (SEQ ID NO: 2) |
| LDHA | GAGATTCCAGTGTGCCTGT | GTCCAATAGCCCAGGATGTG |
| | (SEQ ID NO: 3) | (SEQ ID NO: 4) |
| HK2 | CAAAGTGACAGTGGGTGTGG | CCAGGTCCTTCACTGTCTC |
| | (SEQ ID NO: 5) | (SEQ ID NO: 6) |
| PDK1 | GAAGCAGTTCCTGGACTTCG | ACCAATTGAACGGATGGTGT |
| | (SEQ ID NO: 7) | (SEQ ID NO: 8) |
| ENO1 | GCCGGCTTTACGTTCACCTC | GTTGAAGCACCACTGGGCAC |
| | (SEQ ID NO: 9) | (SEQ ID NO: 10) |
| GLUT1 | CTTCACTGTCGTGTCGCTGT | CCAGGACCCACTTCAAAGAA |
| | (SEQ ID NO: 11) | (SEQ ID NO: 12) |
| G6PD | AAGAACGTGAAGCTCCCTGA | AATATAGGGGATGGGCTTGG |
| | (SEQ ID NO: 13) | (SEQ ID NO: 14) |
| PGD | GGTGCACAACGGGATAGAGT | CCATCGGTGTCTTGGAACTT |
| | (SEQ ID NO: 15) | (SEQ ID NO: 16) |
| RPE | TGGAAAGGATCTGGGAAGTG | CCTGGGGTCAAGATCCATA |
| | (SEQ ID NO: 17) | (SEQ ID NO: 18) |
| RPIA | CTGGATCGACACCCAGAGAT | CGATCACGATGAAGCGACTA |
| | (SEQ ID NO: 19) | (SEQ ID NO: 20) |
| MYC | TGAGGAGACACCGCCCAC | CAACATCGATTTCTTCCTCA |
| | (SEQ ID NO: 21) | TCTTC (SEQ ID NO: 22) |
| PFKP | CTACCAGCGACTTGCCATCA | ATCATAGATGGCGAGCATCC |
| | (SEQ ID NO: 23) | (SEQ ID NO: 24) |
| ACACA | GAGGGCTAGGTCTTTCTGGA | CCACAGTGAAATCTCGTTGA |
| | AG (SEQ ID NO: 25) | GA (SEQ ID NO: 26) |
| ACACB | GCCAGAAGCCCCCAAGAAAC | CGACATGCTCGGCCTCATAG |
| | (SEQ ID NO: 27) | (SEQ ID NO: 28) |
| GPAT | ACTCCTTGGGCCTTTGCTG | TTCTGGAACAGGACCACTGA |
| | (SEQ ID NO: 29) | AGT (SEQ ID NO: 30) |
| DGAT1 | CGTGAGCTACCCGGACAATC | AAAGTTGAGCTCGTAGCACA |
| | (SEQ ID NO: 31) | AGG (SEQ ID NO: 32) |
| PPARG | GAACAGATCCAGTGGTTGCA | GGCATTATGAGACATCCCCA |
| | G (SEQ ID NO: 33) | C (SEQ ID NO: 34) |
| SREBF1 | GGAGAACCTAAGTCTGCGCA | TCCCTCCACTGCCACAGG |
| | CT (SEQ ID NO: 35) | (SEQ ID NO: 36) |
| FASN | TATGCTTCTTCGTGCAGCAG | GCTGCCACACGCTCCTCTAG |
| | TT (SEQ ID NO: 37) | (SEQ ID NO: 38) |
| RICTOR | GCCAAACAGCTCACGGTTGT | CCAGATGAAGCATTGAGCCA |
| | AG (SEQ ID NO: 39) | CTG (SEQ ID NO: 40) |
| RPL19 | ATGCCAGAGAAGGTCACATG | ACACATTCCCCTTCACCTTC |
| | (SEQ ID NO: 41) | (SEQ ID NO: 42) |
| RPS16 | GCTCGCTACCAGAAATCCTA | CATCCAATACCAACACATAA |
| | C (SEQ ID NO: 43) | GGC (SEQ ID NO: 44) |
| ACTB | CTGGAACGGTGAAGGTGACA | AAGGGACTTCCTGTAACAAT |
| | (SEQ ID NO: 45) | GCA (SEQ ID NO: 46) |
| GAPDH | GTCTAGAAAAACCTGCCAAA | CTGTTGAAGTCAGAGGAGAC |
| | TATGA (SEQ ID NO: 47) | CAC (SEQ ID NO: 48) |

P2220810 for mTOR, P130485 for SIN1, and P257029 for G βL were purchased from Bioneer (Daejeon, Korea). qRT-PCR was performed under the following conditions using the SYBR Green Reagent Kit (Applied Biosystems, Foster City, CA, USA) in the ABI PRISM 7700 Sequence Detection System (Applied Biosystems): at 95° C. for 5 minutes, then 40 cycles 94° C. for 10 seconds, 51-55° C. for 10 seconds, 72° C. for 30 seconds.

The reaction was performed in triple execution for each sample and normalized to the level of actin (ACTB) or glyceraldehyde 3-phosphate dehydrogenase (GAPDH).

### Generation of BI-1 (TMBIM6) Knockout Cells by CRISPRICas9 Genome Editing

The CRISPR/Cas9 genome editing method was used to generate the BI-1 (TMBIM6) knockout HT1080 cell line. Plasmids containing sequences targeting human BI-1 were designed and constructed from pRGEN_BI-1 expression vectors by ToolGen (Seoul, Korea). The guide sequence targeting exon 3 of human BI-1 was 5′-TGCAGGGGCCTATGTCCATATGG-3′ (SEQ ID NO: 49).

As a negative control, pRGEN_Scramble vectors were constructed using a scrambled sequence (5′-GCACTACCAGCTAACTCA-3′) (SEQ ID NO: 50) that received information from Origene (# GE100003, pCas-Scramble Vector). pRGEN_BI-1 vector or pRGEN_Scramble mixed with pRGEN_Cas9-CMV and lipofectamine 3000 Using HT1080 and HeLa cells were co-transfected. After 48 hours, the cells were treated with trypsin and laid out on 96-well plates to separate individual clones by the limited dilution method. Cells were cultured for at least 1 week in DMEM containing 10% FBS and antibiotics. The monoclonal was expanded, genomic DNA was purified from the clone and used as a template for PCR-based screening using the following three primers:

| | F1, |
|---|---|
| (SEQ ID NO: 51) | |
| | 5′-CGTTGCTGTGTGGTTATTGG-3′; |
| | R1, |
| (SEQ ID NO: 52) | |
| | 5′-TCAATCCTGCCTCTCCTGAT-3′; |
| | and |
| | target, |
| (SEQ ID NO: 53) | |
| | 5′-TGCAGGGGCCTATGTCCATATGG-3′. |

Knockout clones produced only one PCR product, while regular clones produced two. PCR products of knockout clones were purified using the JETsorb DNA Extraction Kit (Genomed, Leinfelden-Echterdingen, Germany), and deletions were confirmed by sequencing.

### GST Pull-Down Analysis

The GST pull-down analysis is based on the manufacturer's instructions on a commercial kit (21516; Thermo Fisher Scientific). Simply put, GST-RPL19 was expressed in E. coli and purified using GSH bead. The purified protein was bound to the GSH Sepharose column. A soluble lysate (500 μg) of HeLa cells transfected with BI-1-HA or covectors was injected into a GST-RPL19-bound column and stirred at 4° C. for 2 hours. Samples were washed three times with washing buffer, then eluted with elution buffer, separated by SDS-PAGE and then immunoblotting.

### Cancer Research in Animals

Tumor xenografts were performed using Bk1Nbt: BALB/c/nu/nu old mice (electrified multimul) aged 6-8 weeks. The mice were kept in a fully climate-controlled room at a constant temperature and humidity with free access to food and water at a 12 :12 h contrast cycle (n=5/cage).

Animal testing complied with the Chonbuk National University Animal Laboratory Animal Care and Use Committee (Jeonju Laboratory Animal Management and Use Guide, Approval Nos. CBNU 2015-064, CBNU 2016-56, CBNU 2017-002 6, and CBNU 2020-033) and the university's relevant ethics regulations.

In culture, exponentially growing BI-1 (TMBIM6) knockout and WT HT1080 cells were treated with trypsin and quantified through trypan blue staining differentiation, and 3-5×10⁶ cells were resuspended in 0.1 ml PBS. Cells were injected subcutaneously into the flanks of each mouse. 5×10⁶ cells were injected into 0.1 ml PBS for SMAiRNA or BIA treatment. When the weight of the tumor reaches about 100 mg (7-10 days after inoculation), mice are randomly assigned 1 mg/kg BI-1 SAMiRNA diluted in saline, 1 mg/kg BIA diluted in DMSO (final concentration: 10% v/v) or vehicle (saline solution or DMSO, 10% v/v) was divided into groups.

SAMiRNA was administered intravenously in the tail every 3 days and BIA was injected intraperitoneally 5 days per week over 3 weeks. BI-1 SAMiRNA with sequence 5′-AAGGCACUGCAUUGAUCUUU-3′ (SEQ ID NO: 54) and negative control SAMiRNA were obtained from Bioneer.

After 25-28 days, the mice were euthanized, solid tumors were incised, and tumor volumes were recorded. Tumor size was measured with a caliper. The tumor volume (mm³) was calculated by the formula [(shortest diameter)2×longest diameter]/2. Mice were evaluated twice weekly and showed signs of terminal illness, such as paralysis of the hind legs and inability to eat or drink, or were victims of cervical dislocation when sick.

### Immunostaining

Formalin of tissue arrays (BC081120d, PR1921c, CR1001a and BC04002b, Biomax, Rockville, MD, USA) and paraffin-embedded cancers, and normal tissue samples were analyzed by immunohistochemistry. After 6 rounds of paraffinization and rehydration , the tissue Sections were applied to 1× target recovery solution, pH 6.0 (DAKO, Glostrup, Denmark) and then incubated at room temperature with peroxidase blocking solution (DAKO) for 10 minutes. It is then washed with 1× TBST buffer (Scytek Lab, Logan, UT, USA), then washed with protein block (0.25% casein in PBS, DAKO) at room temperature (RT) for 10 minutes and washed at 4° C. with the primary antibody incubated overnight. After rinsing with TBST buffer, sections were incubated with secondary antibodies for 1 hour at room temperature. AEC substrate chromogenic source (DAKO) was added and washed with deionized water. Mayer's Hematoxylin-Aldrich-stained slide counters were rinsed under the tap and mounted using an aqueous medium (Scytek Lab, USA). Ki-67 expression in xenograft tumors was determined as a percentage of positive cells per field and normalized to the total number of cells in each field.

### Immunofluorescence Staining

Cells were injected into Lab-Tek II chamber slides (Thermo Fisher Scientific), rinsed with PBS, fixed in ice-cold methanol for 20 minutes, and washed twice in PBS. Cells were blocked with 0.1% BSA (Sigma-Aldrich) for 30 minutes and then incubated overnight at 4° C. with primary antibodies. After washing, cells were incubated with fluorescein isothiocyanate or tetramethylammonium-conjugated secondary antibody for 1 hour. The specimens were fitted with ProLong Gold anti-fade reagent and stained with 4-, 6-diamidino-2-phenylindole (Invitrogen). Images were obtained from an LSM 510 confocal microscope (Carl Zeiss) and analyzed with AxioVision software.

### Polysomal Profiling and Poly(A) Pull-Down Analysis

Polysome profiles were incubated for 10 minutes with cycloheximide at a final concentration of 100 μg/ml before cells were harvested. The cells were then washed with 100 μg/ml cycloheximide in PBS, collected in tubes, and 1 ml of polysomal lytic buffer (20 mM Tris-HCl pH 7.5, 100 mM NaCl, 10 mM MgCl2, 0.4% IGEPAL, and 10 unit/ml RiboLock RNase inhibitor (E00381, Thermo Scientific) And 100 μg/ml cycloheximide) containing Xpert protease inhibitor cocktail (P3100, genDEPOT, Katy, TX, USA).

After injecting the purified lysate into a 10 ml linear tube formed with a 10-50% (w/v) sucrose gradient (20 mM Tris-HCl pH 7.5, 100 mM NaCl, 10 mM MgCl2, 100 μg/ml cycloheximide, 10 units/ml RNase inhibitor, 1× protease inhibitor cocktail), the P40ST swing rotor (Hitachi, JAPAN) Centrifuged at 36,000 rpm for 2 hours at 4° C. Gradient-fractionation with Fluorinert FC-40 (F9755, Sigma-Aldrich) and 750 μl of fraction were collected in tubes using the ISCO dense gradient fractionation system. For poly(A) pull-down assays, cells are buffered A (50 mM Tris-HCl [pH 7. 4], 100 mM NaCl, 30 mM MgCl2, 0.3% CHAPS, 40 U/ml RNase inhibitor, protease inhibitor cocktail, and 100 μg/ml cycloheximide), and then the lysate is purified at 4° C., 10 min 8000× g, and then oligo (dT) incubated with cellulose (NEB) at room temperature for 1 hour. Oligo (dT) cellulose was pelletized by centrifugation and washed 5 times with buffer A. Purified ribosomal fractions eluted with 100 mM Tris [pH 7.4], 500 mM NaCl, 10 mM EDTA, 1% sodium dodecyl sulfate (SDS) and 5 mM DTT), and bonded and non-binding fractions were dissolved with Vivaspin 500 (Sartorius Stedim).

### Micro-RNA Analysis of HT1080 Cells That Stably Overexpress BI-1

After extracting total RNA from transfected human cells, each RNA sample (30 μg) was converted into cyanine (Cy)3- or Cy5-conjugated dCTP (Amersham, Piscataway, NJ, USA). The labeled cDNA mixture is concentrated with ethanol precipitation and resuspended in 20 μl of hybridization solution (GenoCheck, Daejeon, South Korea), then mixed to form the OpArray Human Genome 35K array (OPHSV4; Operon Biotechnologies, GmbH) and covered with MAUI FL. Incubation at 62° C. for 12 hours in a hybridization chamber (Biomicro Systems, Salt Lake City, UT, USA), then the slides are washed with 2× sodium chloride-sodium citrate (SSC) with 0.1% SDS for 2 minutes, then, washed at room temperature at 1× SSC for 3 minutes and at 0.2× SSC for 2 minutes. The slides were centrifuged and dried at 3000 rpm for 20 seconds. The hybrid slides were scanned with a GenePix 4000B scanner (Axon Instruments, Sunnyvale, CA, USA) and the scanned images were processed with GenePix Pro v.5.1 (Axon Instruments) and GeneSpring GX v.7.3.1 (Silicon Genetics, Redwood City, CA, USA) software. Points judged to be substandard on visual inspection of each slide, including slides with dust artifacts or spatial defects, were excluded from further analysis. Spots with a signal-to-noise ratio of less than 10 were also excluded to filter out unreliable data. Data were normalized to global, lowess, print tips, and extended approaches for data reliability. The fold change filter included a requirement that upregulated genes be present in at least 200% and 50% of the control group, respectively. Data included gene groups that behaved similarly in time-course experiments and were clustered using GeneSpring GX v.7.3.1. We used an algorithm based on Pearson correlation to identify genes with similar patterns.

### Micro-RNA Analysis of BI-1 (TMBIM6) Knockout and WT (WT) HT1080 Cells

Microarray analysis of BI-1 (TMBIM6) knockout and WT HT1080 cells was performed at Ebiogen (Seoul, South Korea). We performed global gene expression analysis using the GeneChip Human Gene 2.0 ST oligonucleotide array (Affymetrix, Santa Clara, CA, USA). Total RNA was isolated using a TRIzol reagent. RNA quality was assessed by a 2100 Bioanalyzer (Agilent Technologies, Santa Clara, CA, USA) and quantified with an ND-1000 spectrophotometer (NanoDrop Technologies, Wilmington, DE, USA), with 300 ng of each RNA sample as input.

Total RNA was converted to double-stranded cDNA. Using a random hexamer containing a T7 promoter, amplified cRNA was generated from a double-stranded cDNA template by in vitro transcription and purified with the Affymetrix sample clearance module. cDNA makes dNTP mixes containing dUTP It was reproduced through random primer reverse transcription using. cDNA was fragmented by uracil DNA glycosylase and purine/pyrimidinic endonuclease 1 restriction endonuclease and terminally labeled with biotinylated dideoxynucleotides in terminal transferase reactions. The fragmented end-labeled cDNA was hybridized into the array for 16 hours at 45° C. and 60 rpm. After hybridization, the array was labeled with streptavidin/phycoerythrin, washed in a GeneChip Fluidics Station 450 (Affymetrix) and scanned using a GeneChip Array scanner 3000 7G (Affymetrix). Image data was extracted from arrays scanned using Command Console v.1.1 software (Affymetrix).

The raw CEL file generated by the above procedure yielded expression intensity data analyzed with the Expression Console v.1.1 software (Affymetrix). To classify co-expressing gene groups with similar expression patterns, we performed using the Multi-Experiment Viewer v.4.4 software. Web-based database tools for annotation, visualization, and integrated discovery are gene ontologies and gene and genomic databases of DEGs classified based on gene function information from the Kyoto Encyclopedia (http://david.abcc.ncifcrf.gov/home.jsp) It was used to interpret functions.

### Bioinformatics and Statistical Analysis

Publicly available gene expression omnibus (GEO) datasets for fibrosarcoma (GSE2719), cervical cancer (GSE63678), breast cancer (GSE31448), lung cancer (GSE19804), and prostate cancer (GSE69223) were used in bioinformatics analysis.

GEO2R was used for BI-1 (TMBIM6) expression analysis. We performed an overall survival analysis of cancer patient samples from the TCGA dataset using the web tools OncoLnc (http://www.oncolnc.org) and GEPIA2 (http://gepia2.cancer-pku.cn).

One-way ANOVA with Tukey post-test, Two-Way ANOVA, Bonferroni post-test, and Student's unpaired t test are Prism v. It was performed using 8 software (GraphPad, San Diego, CA, USA). Data are represented as mean±SD, *p<0.05. **p<0.01; ***p<0.001, ****p<0.0001 were considered statistically significant. The statistical tests used in each case are indicated, and the number of experiments is specified in the legend of each figure.

### Generation of Asthma-Inducing Mice

Asthma-inducing mice were prepared using female WT and BI-1 (TMBIM6) knockout C57BL/6 mice aged 7-8 weeks. The mice were housed at 22±1° C. with a 12-hour contrast period and were freely fed regular feed and water under standard conditions (no specific pathogens) with air filtration. In the saline control (SAL) group and the two groups of OVA/LPS groups (IC87114 or BIA treatment), all OVA/LPS group mice received 75 μg of OVA+10 μg of LPS at days 0, 1, 2, 3, and 7. was sensitized into the nasal cavity and challenged with 50 μg of OVA alone on days 14, 15, 21 and 22.

### Bronchoalveolar Lavage Fluid BAL Fluid Sample Analysis

A sample of bronchoalveolar lavage fluid (BALF) (1 ml) was obtained from each mouse. Samples were centrifuged (600 g, 3 min) and supernatants were stored at −20° C. for cytokine analysis. Cell pellets in the sample were pooled for total cell count using model Z1 (Beckman-Coulter, Miami, Florida, USA) after erythrocyte lysis (Zap-Oglobin II, Beckman-Coulter, Fullerton, CA, USA).

Cells were loaded into slides, centrifuged (700× g, 3 min) and stained with Diff-Quick (Baxter, Detroit, MI, USA). The difference was confirmed through an optical microscope.

### Measurement of Airway Hypersensitivity Reactions

Airway hypersensitivity reactions were evaluated by systemic volumetry during airflow disturbance induced by methacholine (MeCh) aerosols. Each group of mice was exposed to the aerosolized saline solution for 3 minutes and then exposed to an increase in the concentration of aerosolized MeCh. The exposed 12 mg/ml, 25 mg/ml and 50 mg/ml MeCh were dissolved in an isotonic saline solution.

Each dose was sprayed for 2 minutes and airway responses were recorded for 5 minutes. Enhanced pauses (Penh) were recorded for 3 minutes after each administration through the main chamber opening. Penh values measured during each 3-minute sequence were averaged for each dose. Penh data showed a change from baseline per dose of MeCh. The percentage increase in Penh compared to baseline at each concentration was used to compare airway reactivity between experimental groups.

### Example <1> Analysis of BI-1 (TMBIM6) Expression in Tumor Samples

We analyzed BI-1 mRNA expression profiling datasets for several tumor samples provided by NCBI's Gene Expression Omnibus (GEO) to investigate the carcinogenic role of BI-1 in cancer progression.

Publicly available gene expression omnibus (GEO) datasets for fibrosarcoma (GSE2719), cervical cancer (GSE63678), breast cancer (GSE31448), lung cancer (GSE19804), and prostate cancer (GSE69223) were used in bioinformatics analysis.

GEO2R was used for BI-1 (TMBIM6) expression analysis. We performed overall survival analysis of cancer patient samples on TCGA datasets using the web tools OncoLnc (http://www.oncolnc.org) and GEPIA2 (http://gepia2.cancer-pku.cn).

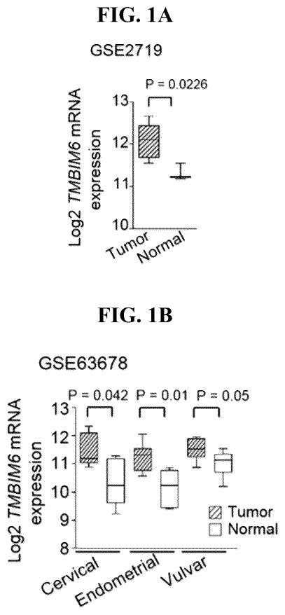

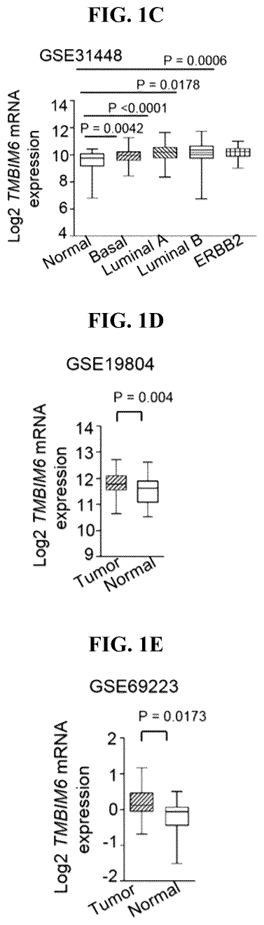

This analysis revealed that BI-1 was significantly overexpressed in fibrosarcoma, cervical cancer, endometrial cancer and vulvar cancer, breast cancer, lung cancer and prostate cancer (FIGS. 1A-1E).

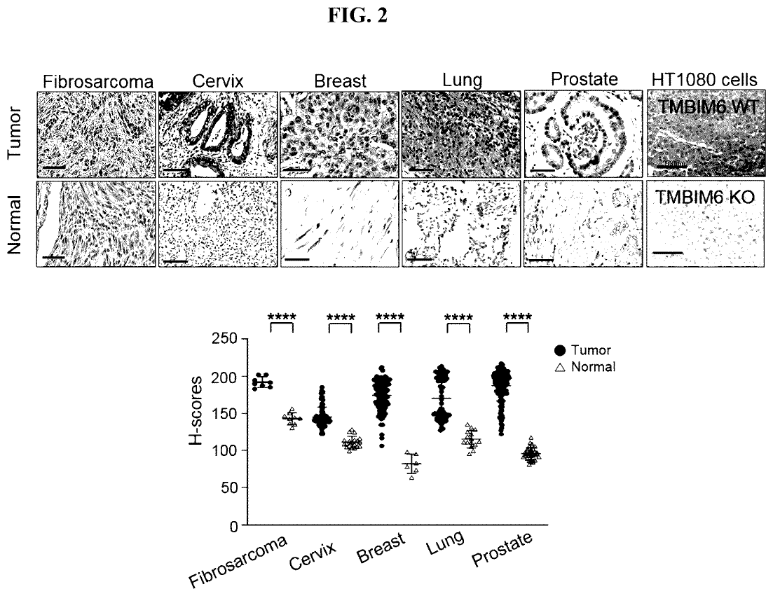

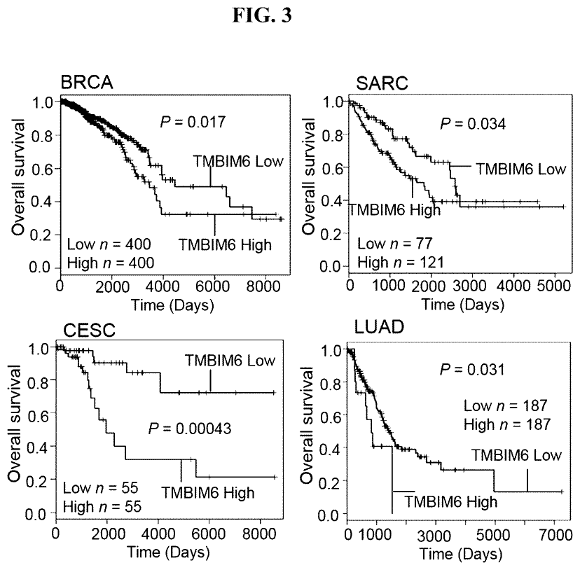

Next, similar results were obtained by comparing the expression level of BI-1 by immunohistochemical staining in the same cancer tissue using a tissue microarray (FIG. 2).

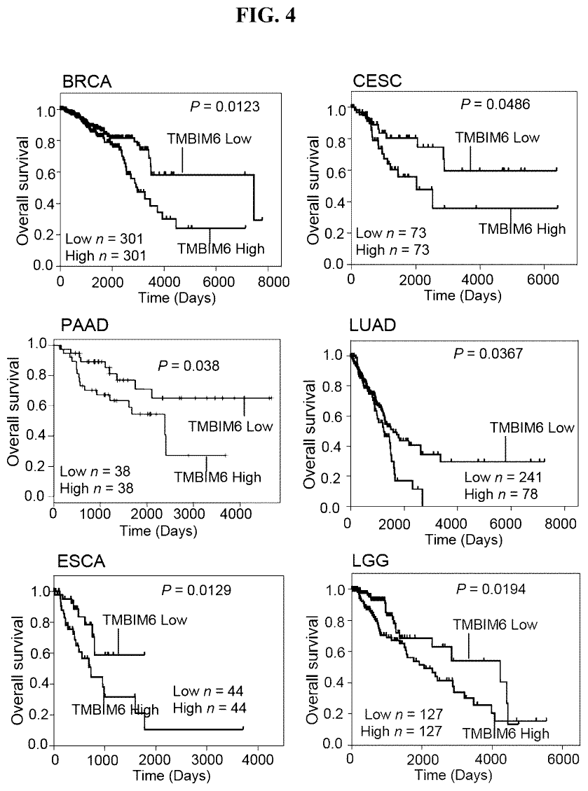

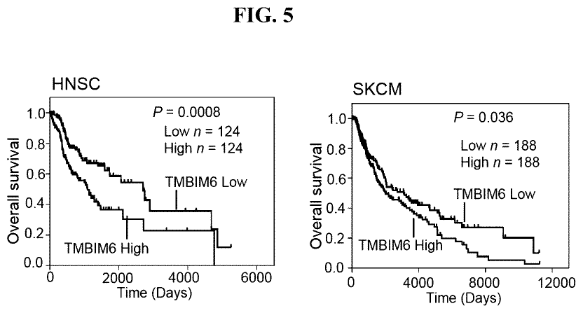

To further investigate whether the level of BI-1 (TMBIM6) expression in tumors is associated with prognosis, we analyzed the correlation between BI-1 (TMBIM6) expression and overall survival (OS) using GTEx project 32 and OncoLnc in TCGA2 and TCGA3 3. The analysis found that patients with high BI-1 (TMBIM6) expression had poor survival in breast invasive carcinoma (BRCA), cervical squamous cell carcinoma and cervical adenocarcinoma (CESC), sarcoma (SARC) and lung adenocarcinoma (LUAD). These data suggest that BI-1 has potential clinical value as a predictive biomarker for disease outcomes in multiple cancers. In addition, high BI-1 expression was confirmed in several cancers, including pancreatic adenocarcinoma (PAAD), esophageal carcinoma (ESCA), cutaneous melanoma (SKCM), head and neck squamous cell carcinoma (HNSC), and intracerebral glioma (LGG) (FIGS. 3 to 5).

### Example <2> Preparation of BI-1 (TMBIM6) Knockout Cells

BI-1080 (TMBIM1) knockout (knockout) cells were created using CRISPR/Cas6 technology to target HT6 and HeLa cell lines.

Plasmids comprising sequences targeting human BI-1 were designed and constructed from pRGEN BI-1 expression vectors by ToolGen (Seoul, Korea).

The guide sequence targeting exon 3 of human BI-1 was 5′-TGCAGGGGCCTATGTCCATATGG-3′ (SEQ ID NO: 55).

As a negative control, the pRGEN_Scramble vector was constructed using a scramble sequence (5′-GCACTACCAGAGGCTAACTCA-3′) (SEQ ID NO: 56) that received information from Origene (#GE100003, pCas-Scramble Vector).

pRGEN BI-1 vectors or pRGEN_Scramble were mixed with pRGEN _Cas9-CMV and co-transfected with HT1080 and HeLa cells using lipopectamine 3000. After 48 hours, treat the cells with trypsin and isolate the individual clones by the limited dilution method Laid on 96 well plates. Cells were cultured for at least 1 week in DMEM containing 10% FBS and antibiotics. The monoclonal was extended and genomic DNA was purified from the clone and used as a template for PCR-based screening using the following three primers:

| | F1, |
|---|---|
| (SEQ ID NO: 57) | |
| | 5′-CGTTGCTGTGTGGTTATTGG-3′; |
| | R1, |
| (SEQ ID NO: 58) | |
| | 5′-TCAATCCTGCCTCTCCTGAT-3′; |
| | and |
| | target, |
| (SEQ ID NO: 59) | |
| | 5′-TGCAGGGGCCTATGTCCATATGG-3′. |

Knockout clones produced only one PCR product, while regular clones produced two. PCR products of knockout clones were purified using the JETsorb DNA Extraction Kit (Genomed, Leinfelden-Echterdingen, Germany), and deletions were confirmed by sequencing.

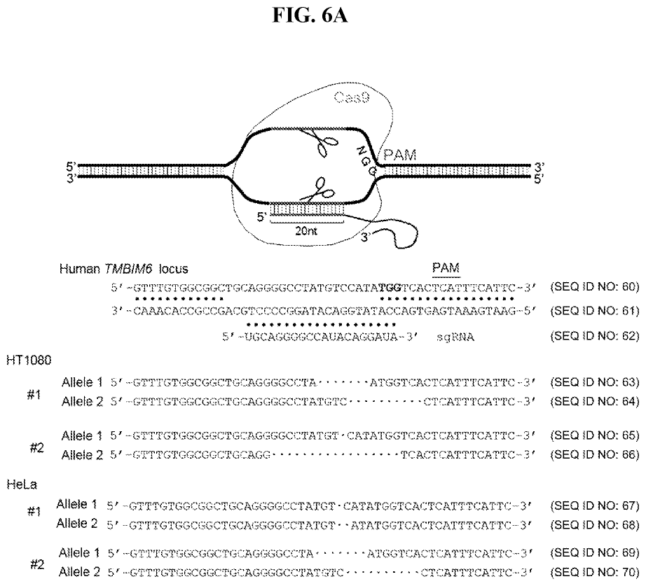

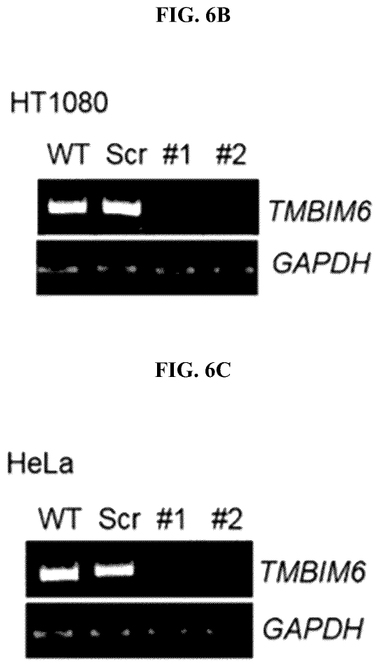

FIG. 6A shows the preparation of BI-1 (TMBIM6) knockout cells using CRISPR/Cas9 genome editing techniques. FIG. 6A is a schematic representation of genomic editing that uses sgRNA (SEQ ID NO: 62) with CRISPR/Cas9 technology to cut DNA site-specifically. It shows mutated allele sequences of BI-1 (SEQ ID NOS: 60 and 61), including insertion/deletion in HT1080 cells (shown by alleles 1 and 2 of HT1080 nos. 1 and 2, represented by SEQ ID NOS: 63-66) and HeLa cells (shown by alleles 1 and 2 of HeLa nos. 1 and 2, represented by SEQ ID NOS: 67-70).

FIG. 6B and FIG. 6C detect mRNA levels of BI-1 in HT108 cells and HeLa cells knocked out of WT and BI-1 by qRT-PCR, respectively. This is the result of one of two experiments that achieved similar results.

### Example <3> Inhibition of Tumorigenicity of Cancer by Knock-Out, Deficient of BI-1

Cell proliferation, migration and invasion analyses were performed to see the effect of BI-1 on cancer proliferation.

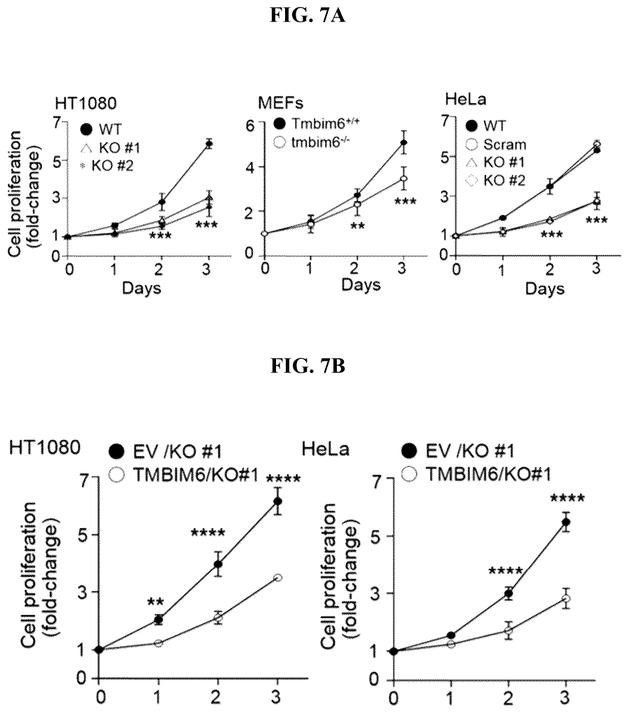

HT1080, HeLa cells , and mouse embryonic fibroblasts (MEF) with BI-1 knocked out all show slower growth compared to WT (WT) cells (see FIG. 7A), while BI-1 (TMBIM6) knockout cells reexpressing BI-1 have a growth rate It was restored (see FIG. 7B).

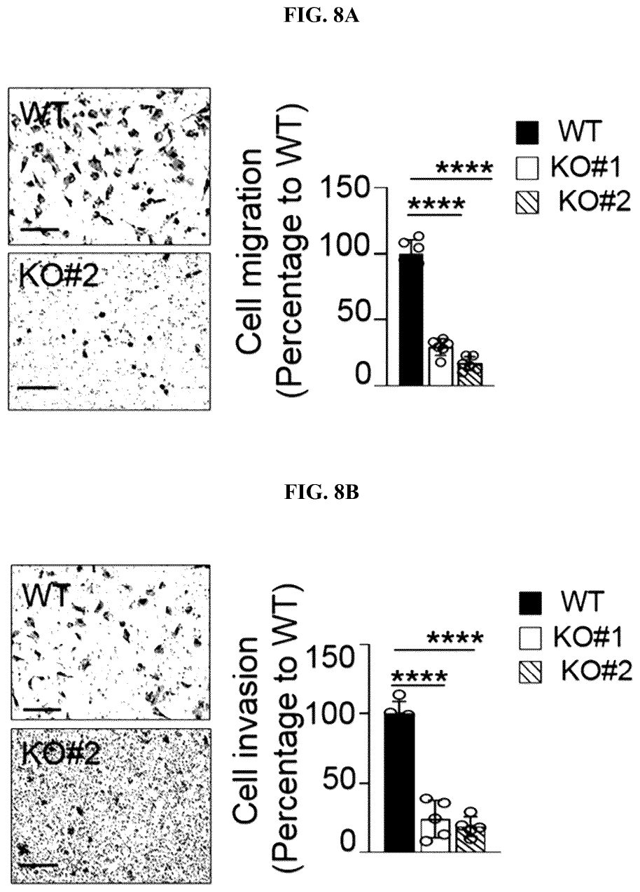

In cells lacking BI-1, cell migration (see FIG. 8A) and invasion (see FIG. 8B) were inhibited.

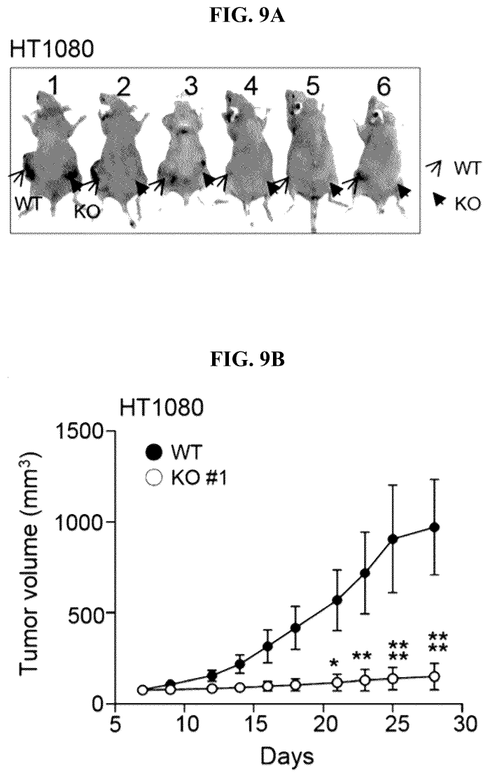

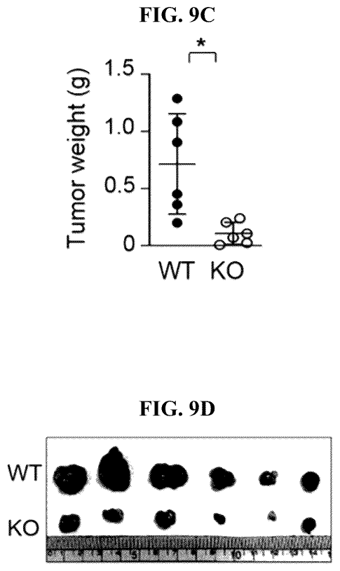

To investigate the role of BI-1 in tumor cell growth in mice, BI-1 (TMBIM6) WT (WT) and knockout HT1080 cells were injected subcutaneously on the left and right sides of immunocompromised mice. Tumor formation and tumor weight arising from BI-1 (TMBIM6) knockout HT1080 cells were significantly reduced compared to WT cells (see FIGS. 9A-9D).

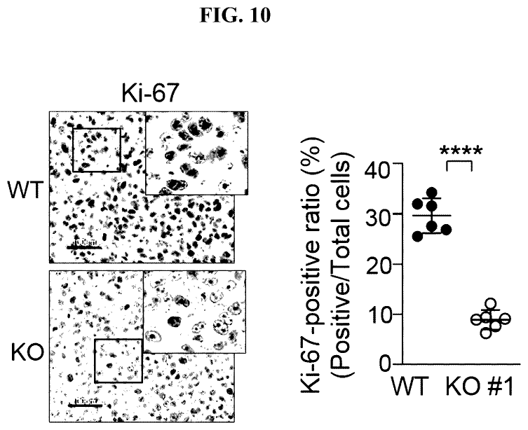

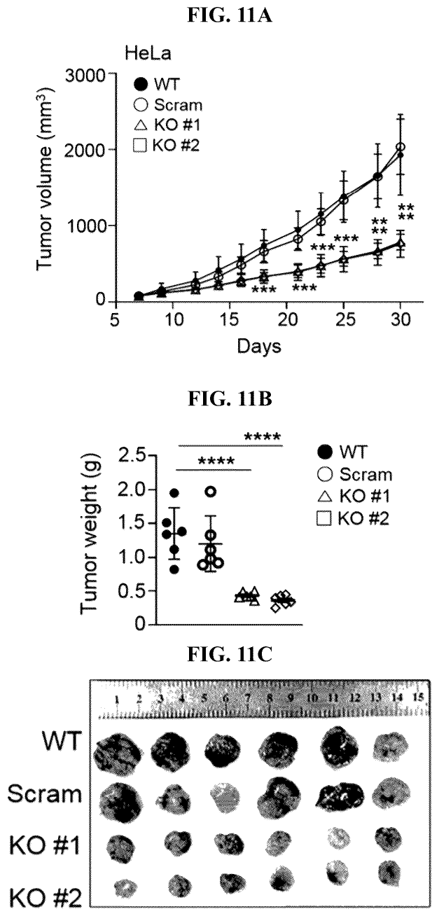

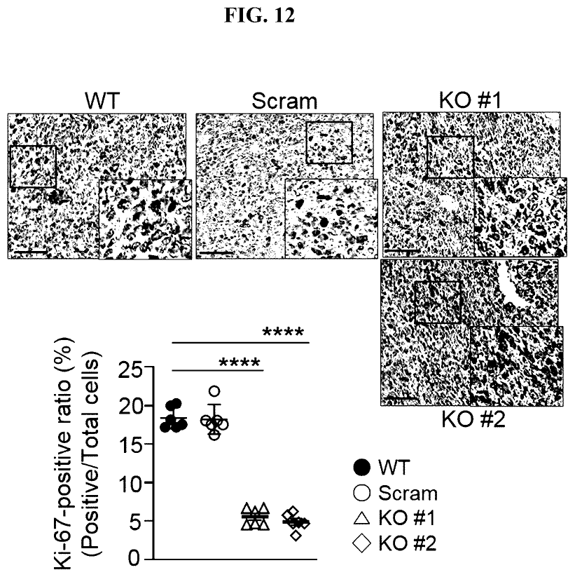

Immunohistochemical staining of Ki67-positive proliferative cells showed that the proliferation of xenograft tumors from BI-1 (TMBIM6) knockout cells was significantly reduced (see FIG. 10). Consistently, BI-1 (TMBIM6) knockout HeLa cells also clearly reduced tumor formation, weight, and expression of Ki-67 than in WT cells (see FIGS. 11 and 12).

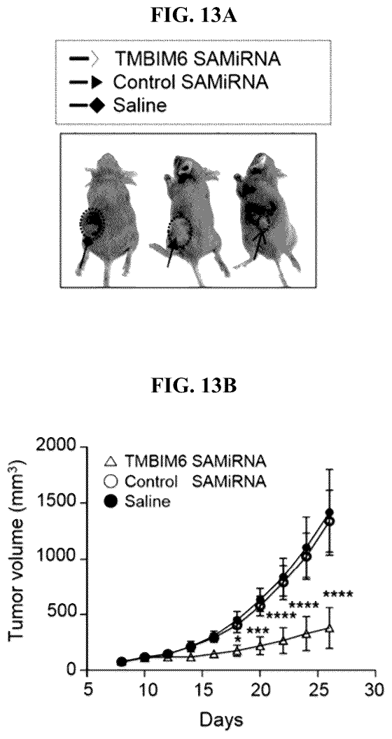

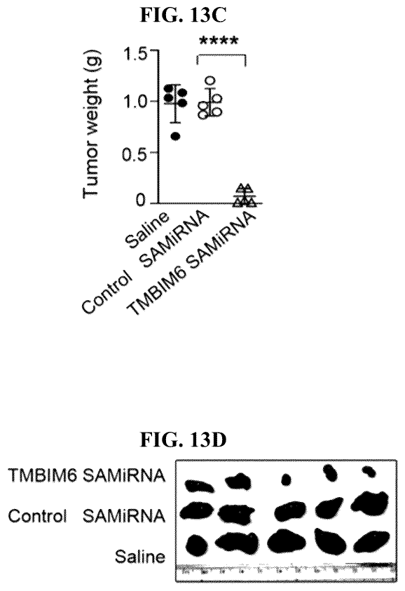

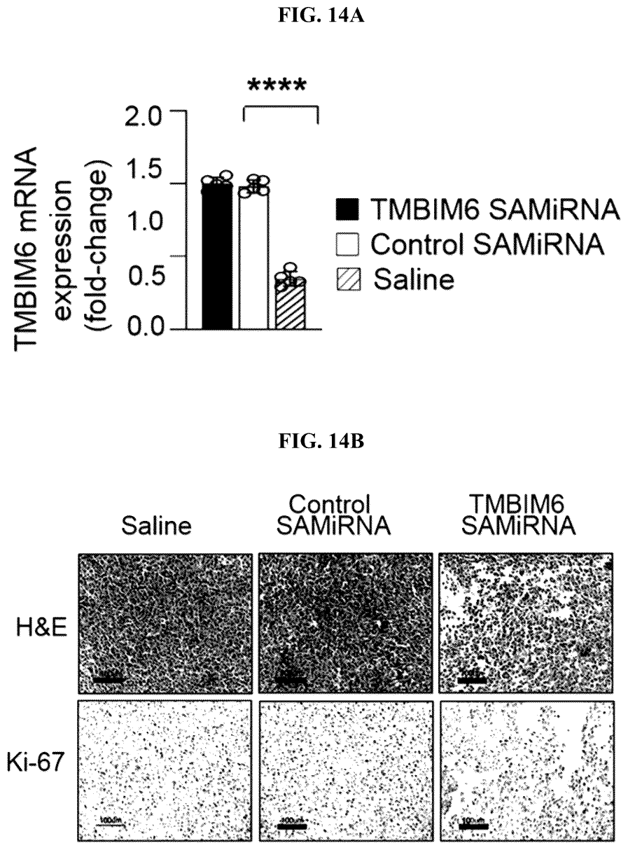

In addition, tumor formation and Ki6 7 expression were reduced even under BI-1 (TMBIM6) knockdown conditions injected with SAMiRNA (self-assembled micelle inhibitory RNA), a stable siRNA silencing platform for efficient in vivo targeting of genes (FIGS. 13A-14B).

The results of in vitro and in vivo experiments such as those above suggest that BI-1 promotes tumor growth.

### Example <4> AKR Path Activation of BI-1 Via mTORC2-Ribosome Axis

Protein phospho-kinase profiling assays were performed to evaluate signaling protein molecules that regulate cancer progression in WT (WT) and BI-1 (TMBIM6) knockout (KO) HT1080 cells.

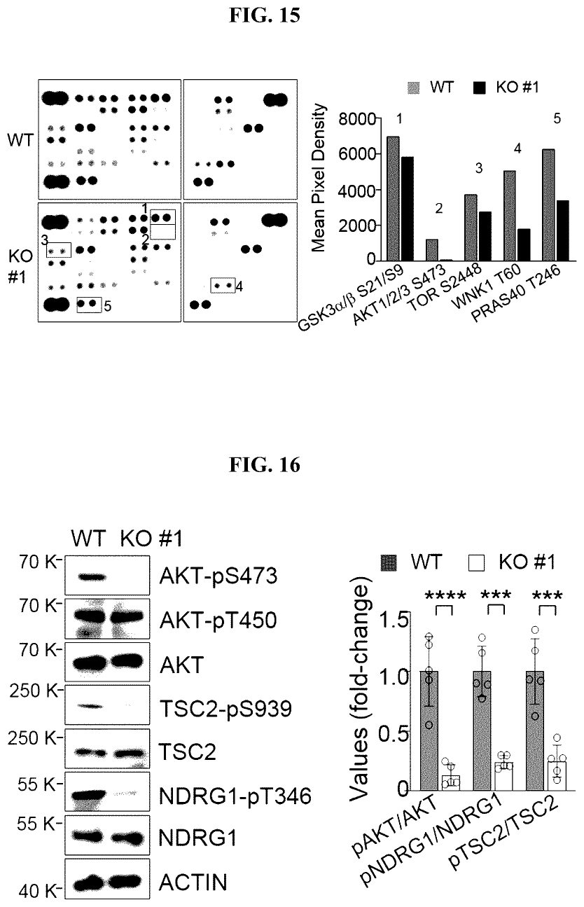

The results showed that phosphorylation of AKT (pAKT-S473), PRAS40, mTOR, GSK3-α/β, and WNK1 was reduced in BI-1 (TMBIM6) knockout HT1080 cells (see FIG. 15).

Since PRAS40, GSK3-α/β, and WNK1 are known substrates of AKT, we looked at whether mTORC2, the AKT higher signaler, was altered in BI-1 (TMBIM6) knockout cells. Removal of BI-1 reduced phosphorylation of mTORC2, AKT and NDRG1 and reduced TSC2 as an AKT substrate (see FIG. 16).

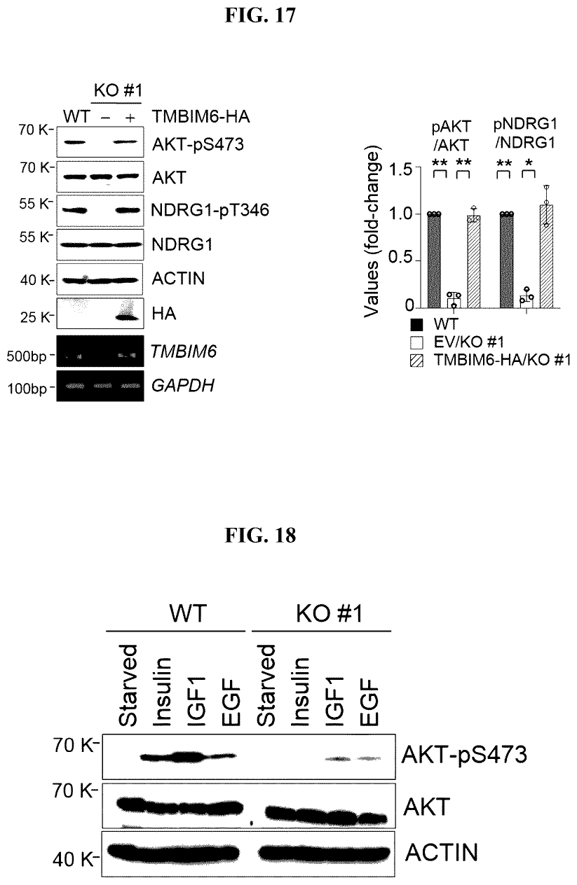

Reintroduction of BI-1 into BI-1 (TMBIM6) knockout HT1080 cells restored phosphorylation of AKT (pAKT-S473) and NDRG1 (pNDRG1-S939) (see FIG. 17).

Upon stimulation of insulin, IGF1 or EGF after starvation of serum, phosphorylation of AKT was induced higher in WT cells compared to BI-1 (TMBIM6) knockout cells (see FIG. 18).

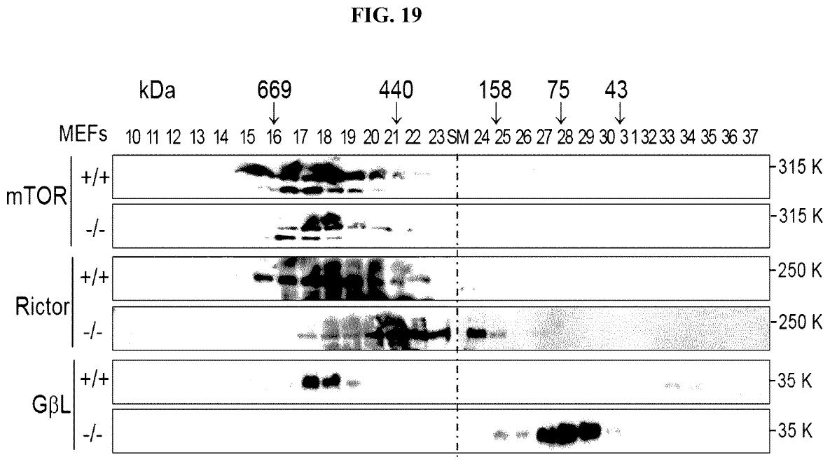

The assembly of mTORC2 and its association with ribosomes are closely related to AKT phosphorylation and were therefore evaluated in BI-1 (TMBIM6) knockout cells. Gel filtration assay with MEF showed that mTORC2 was downregulated by the deletion of BI-1 (see FIG. 19).

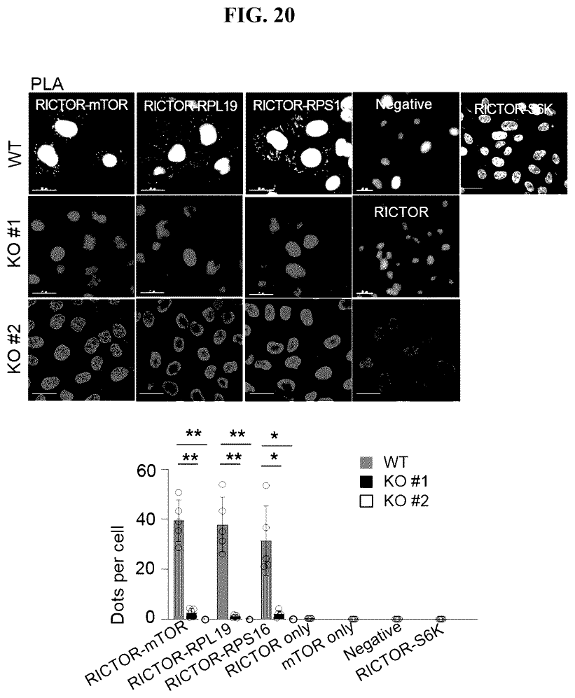

In PLA analysis (in situ proximity ligation assay), the interaction between RCTOR and mTOR, RPL19 and RPS16 was significantly reduced in BI-1 (TMBIM6) knockout HT1080 and HeLa cells (see FIG. 20).

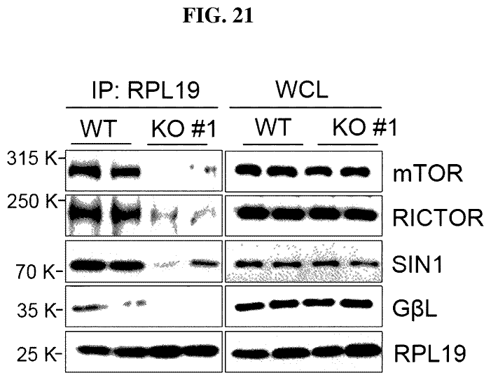

In an immunoprecipitation (Co-IP) assay using RPL19, the binding of mTOR, RICTOR, SIN1 and Gβ L (also known as mLST8) to RPL19 was mostly removed from knockout cells (see FIG. 21). Moreover, the expression levels of proteins and mRNA of these genes were the same in BI-1 (TMBIM6) WT and knockout cells (see FIG. 21).

The results of the above disclosure suggest that BI-1 is one of the essential genes for mTORC2 signaling that regulates AKT activity.

### Example <5> mTORC2 Activation Control of BI-1

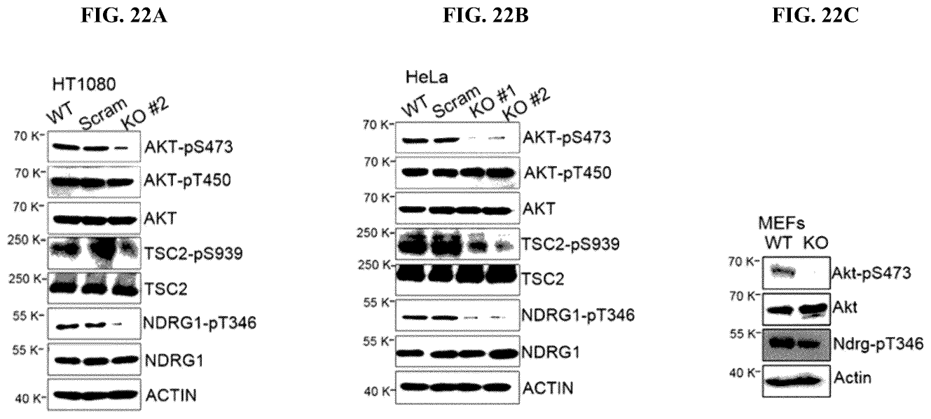

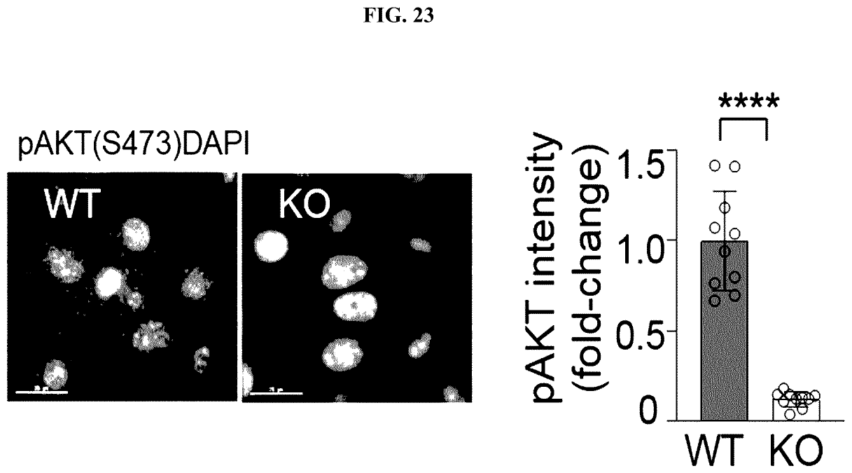

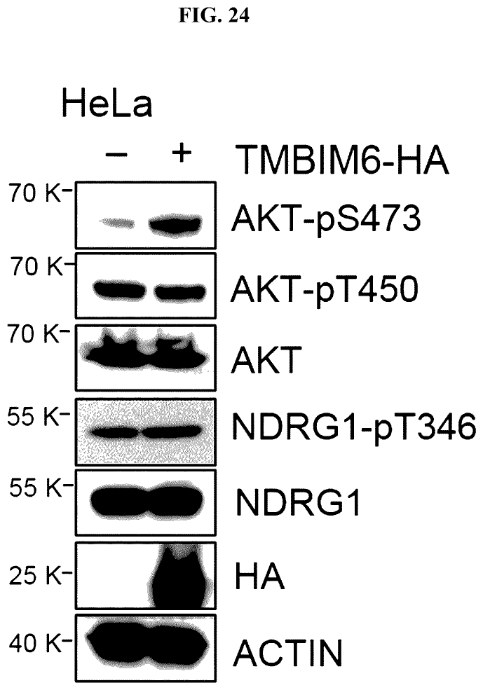

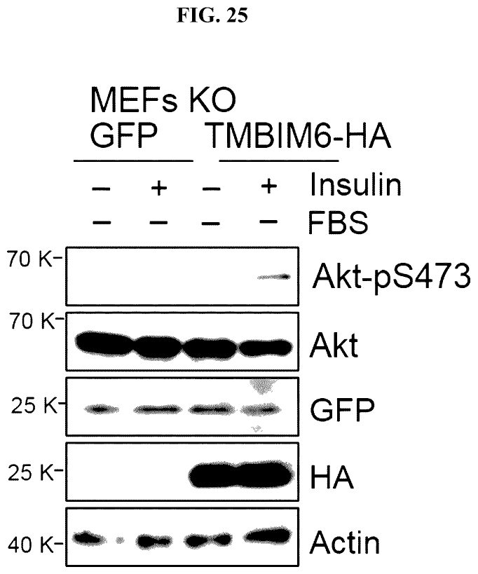

We investigated whether mTORC2, the upper regulator of AKT, varies in BI-1 (TMBIM6) knockout cells. Under BI-1 (TMBIM6) removal conditions, phosphorylation of AKT and NDRG1 as mTORC2 substrates was reduced, and phosphorylation of TSC2 as AKT substrates was reduced (see FIGS. 22A-22C). Immunofluorescence staining showed that phosphorylation of AKT was reduced by BI-1 (TMBIM6) deletion (see FIG. 23). Consistent with the above results, overexpression of BI-1 in HeLa cells increased mTORC2 activity (FIG. 24). Phosphorylation of AKT (pAKT-S473) in BI-1 (TMBIM6) knockout MEF cells (MEF−/−) with BI-1-HA overexpression increased during insulin stimulation after serum starvation (see FIG. 25).

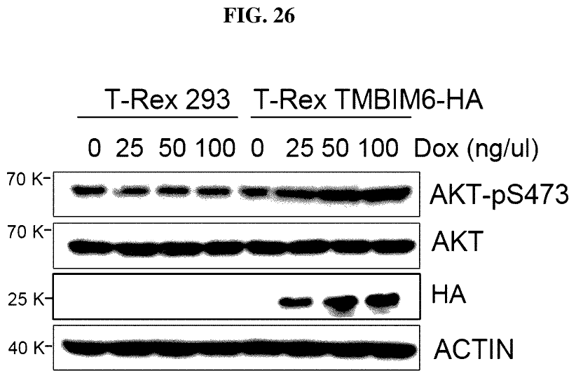

To further investigate whether AKT phosphorylation is BI-1-dependent, stable T-Rex-293 cells with tetracycline-induced BI-1 (TMBIM6) expression were established. BI-1 (TMBIM6) levels were increased by doxycycline treatment in a dose-dependent manner with a concomitant increase in AKT phosphorylation (see FIG. 26), suggesting that BI-1 is one of the essential genes for mTORC2 signaling that regulates AKT activity.

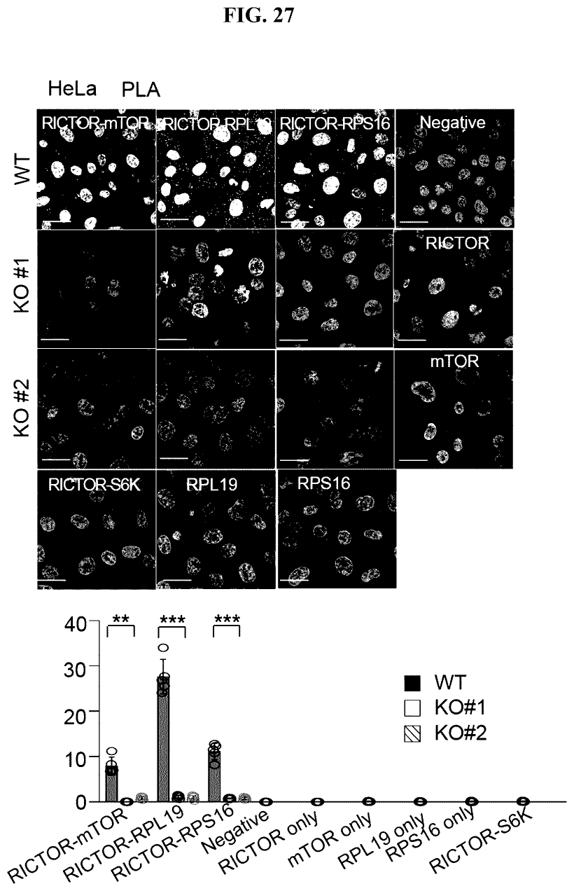

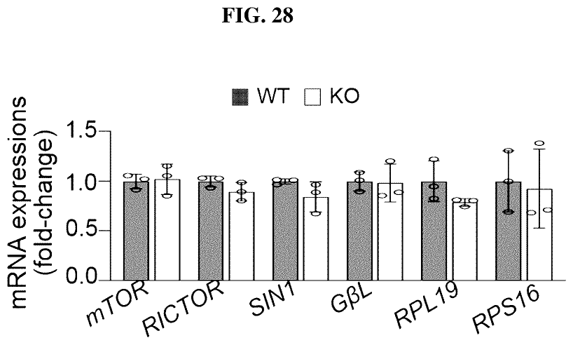

In PLA (in situ proximity ligation assay), the interaction between RICTOR and mTOR, RPL19 and RPS16 was significantly reduced in BI-1 (TMBIM6) knockout HT1080 and HeLa cells (see FIG. 27). The expression levels of proteins and mRNA of these genes were the same in BI-1 (TMBIM6) WT and knockout cells (see FIG. 28).

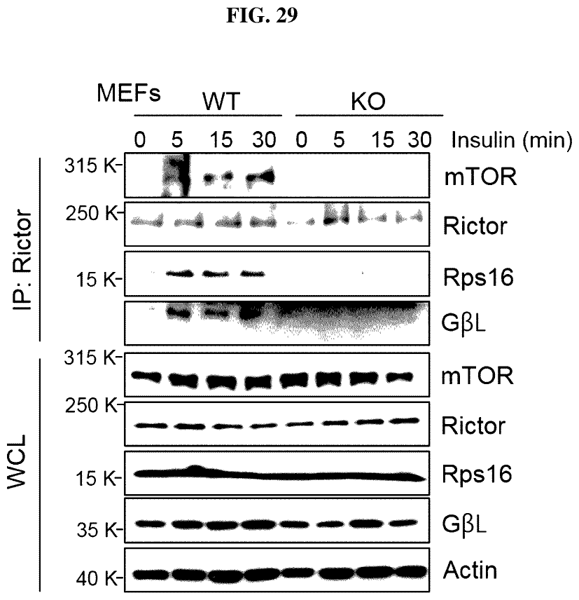

Co-IP assay with anti-RICTOR antibodies to insulin stimulation after serum starvation was performed to determine whether the binding between mTORC2 and ribosomes was BI-1 dependent. Anti-RICTOR antibodies were pulled down to mTOR, GfβL and RPS16 in WT cells, but not in BI-1 (TMBIM6) knockout cells (see FIG. 29).

### Example <6>Association regulation of mTORC2 and ribosomes of BI-1

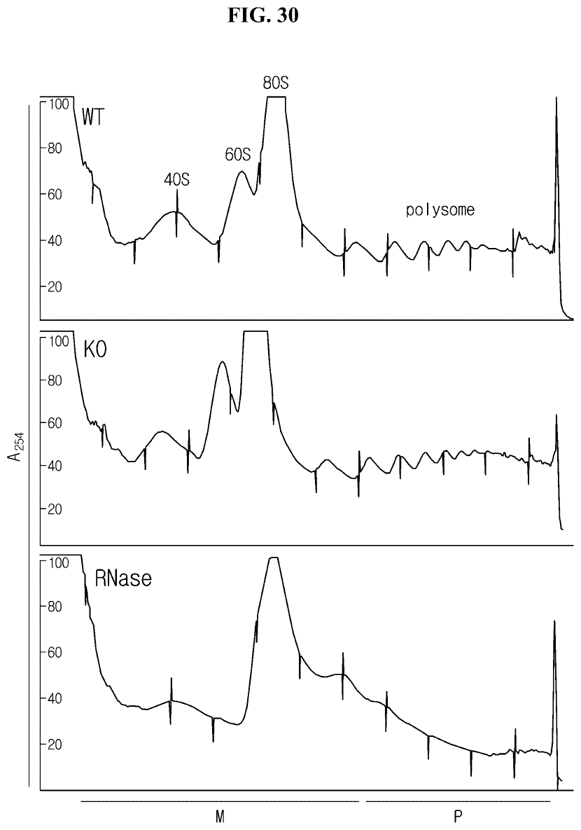

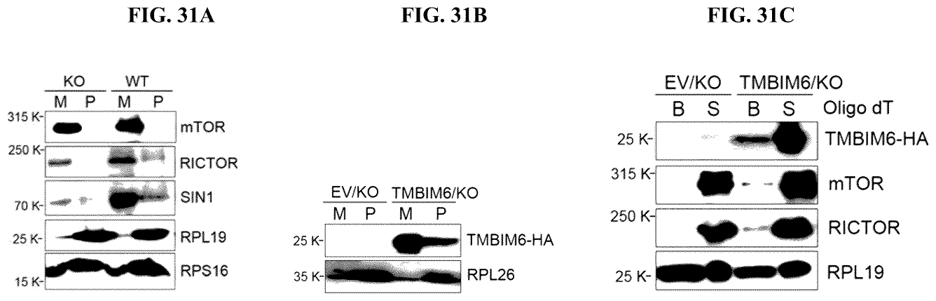

To determine whether reducing mTORC2 activity in BI-1 (TMBIM6) knockout cells was associated with impairment of ribosomal maturation, polysomes were isolated from 80S, 60S and 40S ribosomes through fractionation. The pattern of ribosomal profiling was the same between BI-1 (TMBIM6) WT and knockout cells, indicating that BI-1 is not associated with ribosomal maturation (see FIG. 30). However, mTORC2 components were relatively less detected in polysomes and ribosome fractions of BI-1 (TMBIM6) knockout HT1080 cells compared to components of WT cells (see FIG. 31A). In addition, BI-1 was co-purified with polysomes and ribosomal fractions in cells under BI-1 (TMBIM6) rescue conditions (see FIG. 31B). mTORC2 physically interacts with translation (mRNA binding) and untranslated Since 80S ribosomes 29 and BI-1 bind to mTORC2, we wanted to find out whether BI-1 co-purifies with mTORC2 in mRNA-bound ribosomes. In mRNA-bound ribosomes purified by pulldown of poly (A) mRNA with oligo (dT) cellulose, BI-1 was co-purified with mTOR, RICTOR and RPL19 (see FIG. 31C). These results suggest that BI-1 regulates the assembly of mTORC2 components and promotes a physical association between mTORC2 and ribosomes.

### Example <7> mTORC2 Residency Regulation on the Endoplasmic Reticulum of BI-1

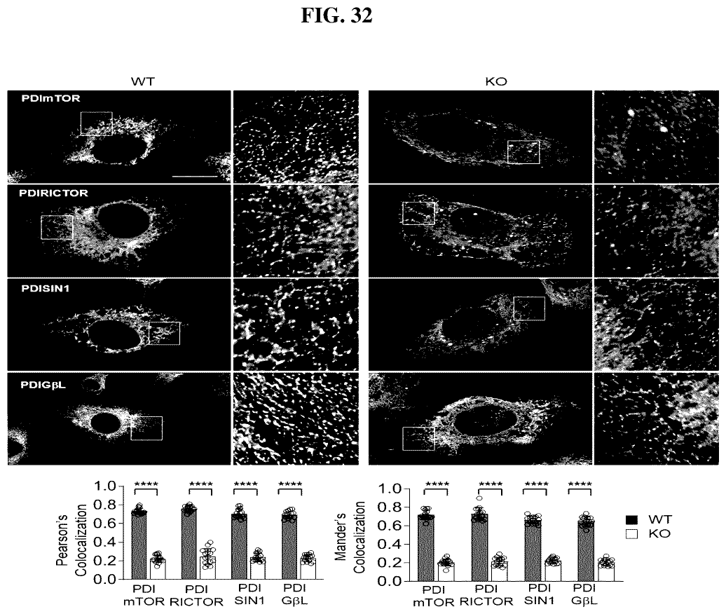

mTORC2 interacts with ER-bound ribosomes on the endoplasmic reticulum (ER) membrane, which is necessary for kinase activity. Immunofluorescence assays were performed to determine if localization of mTORC2 in ER was different between BI-1 (TMBIM6)WT and knockout cells. Co-localization of the ER marker protein PDI (protein disulfide isomerase) and mTORC2 components was also reduced in BI-1 (TMBIM6) knockout cells (see FIG. 32), which indicates that BI-1 is the residence of mTORC2 in ER (residency).

### Example <8> AKT-Dependent Metabolic Modulation of BI-1

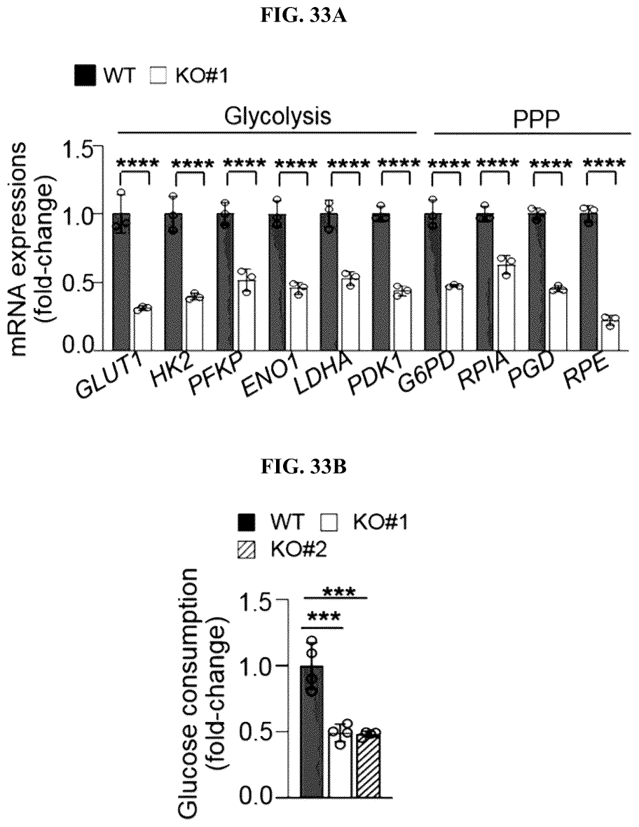

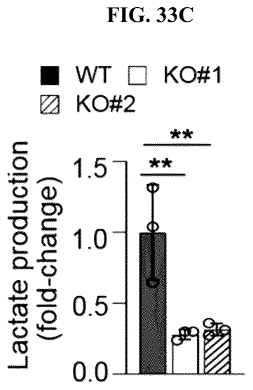

mTORC2 regulates cellular bioenergy by regulating glycolytic gene expression, aerobic glycolysis, glutathione (GSH) biosynthesis, hexosamine biosynthesis pathway (HBP) and glycosylation. BI-1 (TMBIM6) knockout cells showed downregulation of glycolysis genes (see FIG. 33A) and also reduced glucose consumption and lactate production (see FIG. 33B and FIG. 33C).

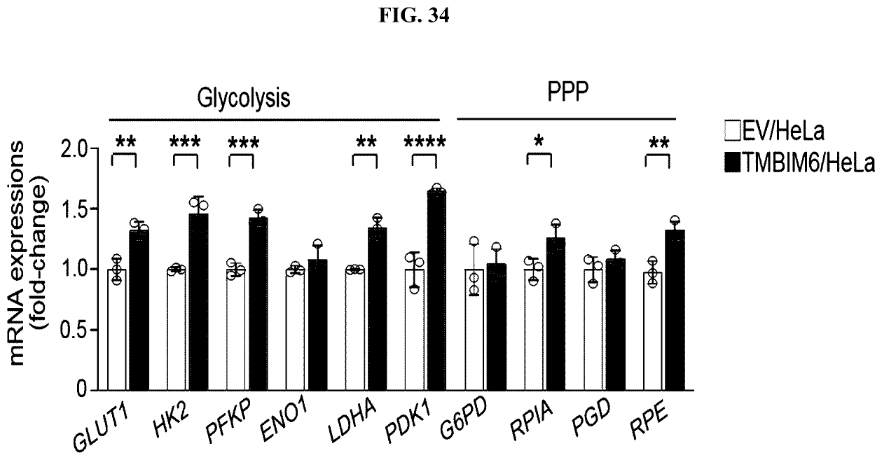

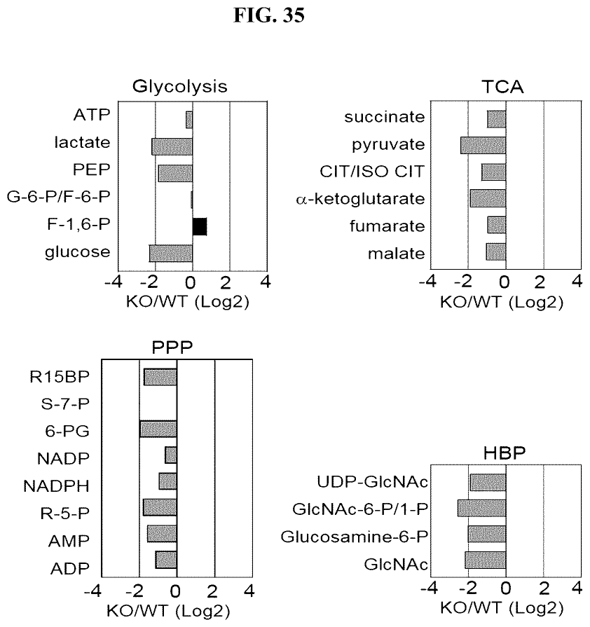

Expression of genes associated with the 5-tantose phosphate pathway (PPP) was also reduced in BI-1 (TMBIM6) knockouts, which were reversed in BI-1 (TMBIM6) and expressed HeLa cells (see FIG. 34). MS analysis showed that metabolite levels of glycolysis, tricarboxylic acid cycle (TCA), PPP and HBP in BI-1 (TMBIM6) knockout cells were reduced compared to WT cells (see FIG. 35). This suggests that the metabolic pathway is associated with the inhibition of BI-1, mTORC2 activity.

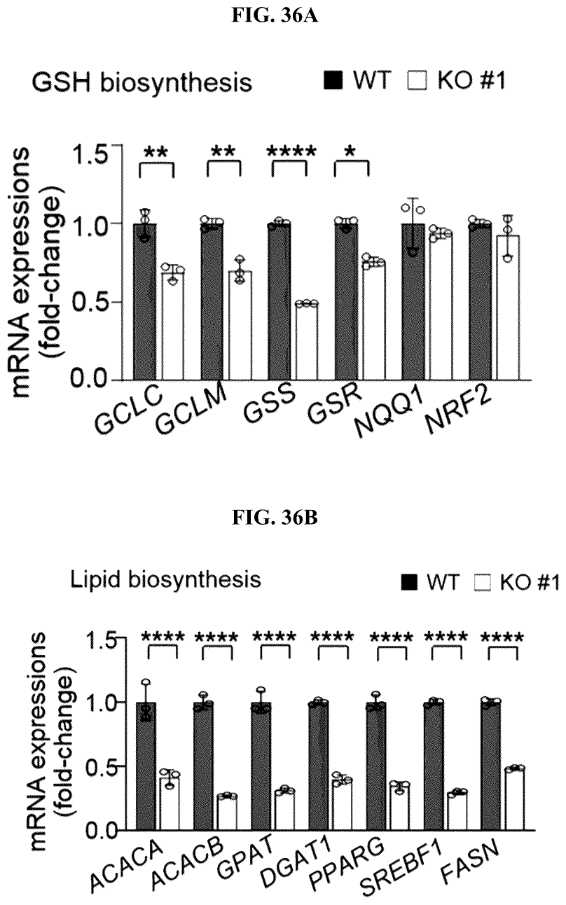

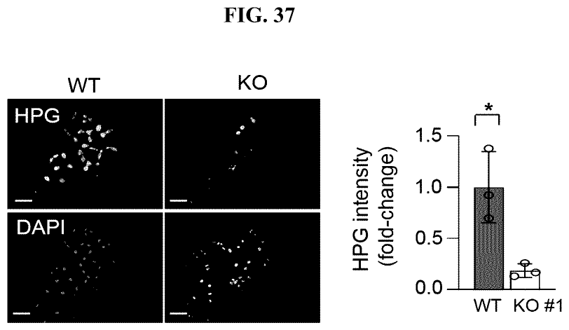

Next, we analyzed the expression of genes involved in AKT-related GSH biosynthesis, de novo lipogenesis, and protein synthesis. BI-1 (TMBIM6) knockout cells showed reduced expression of GCLC, GCLM, GSS and GSR (see FIG. 36A), denovo lipogenesis including SREBF1 necessary for cholesterol, fatty acids, triglycerides, phospholipid synthesis and The expression of the gene involved was also reduced in these cells (see FIG. 36B). Overall, protein synthesis was significantly reduced due to the loss of BI-1 (see FIG. 37).

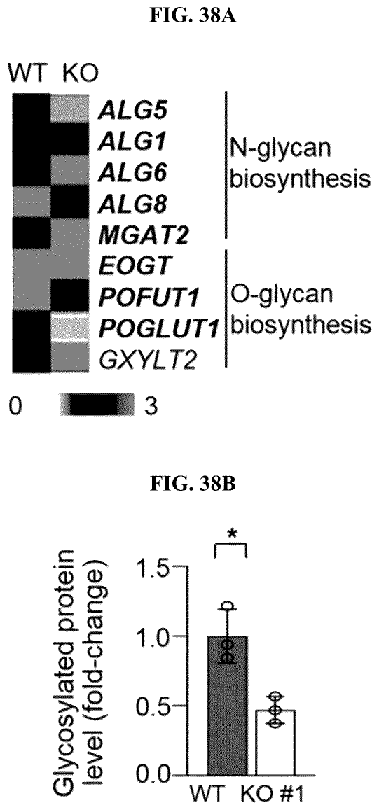

Investigation of glycoprotein folding status showed that the basal level of glycated protein was lower in the BI-1 (TMBIM6) knockout than in WT HT1080 cells (see FIG. 38A). Microarray analysis data showed a decrease in the expression of glycosylating gene including ALGS, ALG1, ALG6, ALG8, MGAT2, EOGT, POFUT1 and POGLUT1 in BI-1 (TMBIM6) knockout cells (see FIG. 38B). These results indicate that BI-1 regulates mTORC2 activity by altering signaling in metabolism.

### Example <9> BI-1's Direct Interaction With mTORC2 and Ribosomes

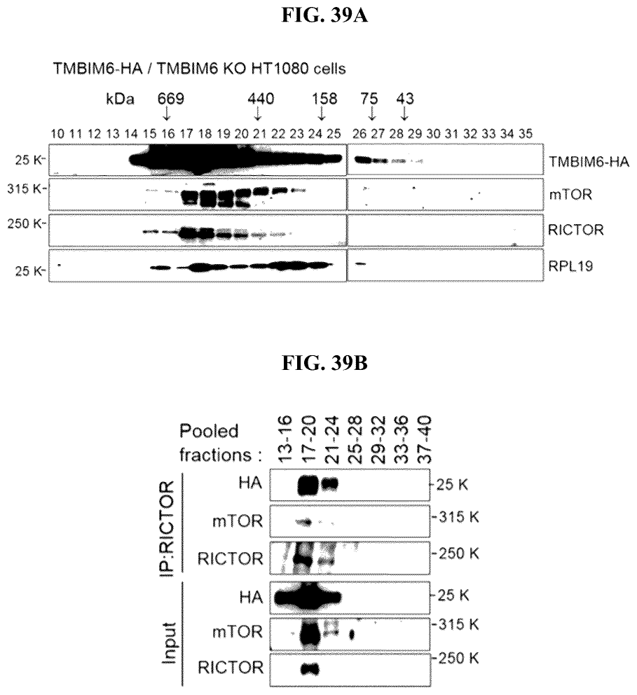

By transfecting BI-1 (TMBIM6) knockout HT1080 cells with HA-tagged BI-1 (BI-1-HA), we investigated whether BI-1 directly interacts with mTORC2 components and ribosomes. Gel filtration analysis and immunoprecipitation of pooled samples using anti-RICTOR antibodies demonstrated that BI-1 is directly bound to mTORC2 (see FIGS. 39A and 39B).

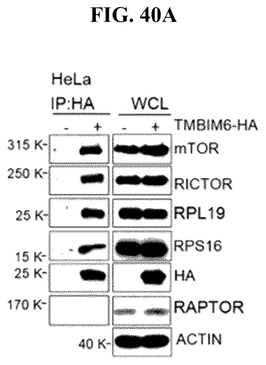

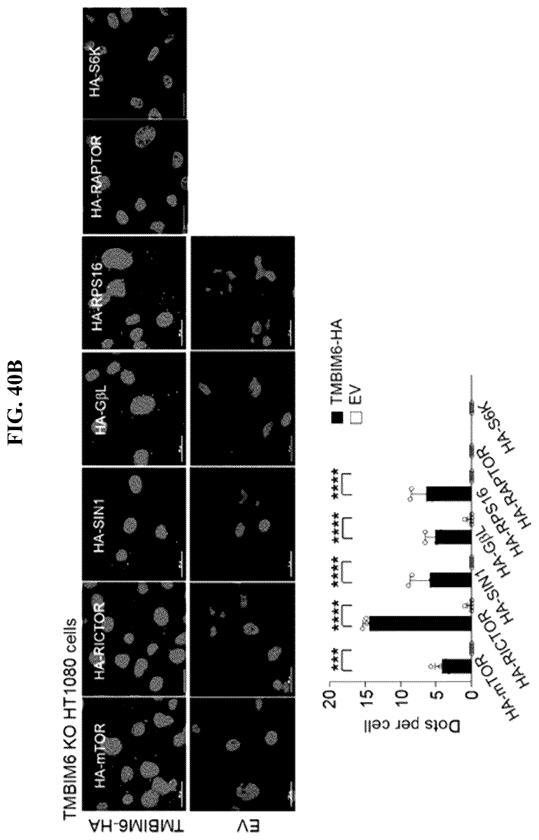

Moreover, the association between BI-1 and endogenous mTORC2 or ribosomes (60S RPL19 and 40S RPS16) in BI-1-HA-overexpressed HeLa and HT1080 cells was confirmed by immunoprecipitation and PLA analysis that it was not related to RAPTOR as a mTORC1 subunit. (See FIGS. 40A and 40B).

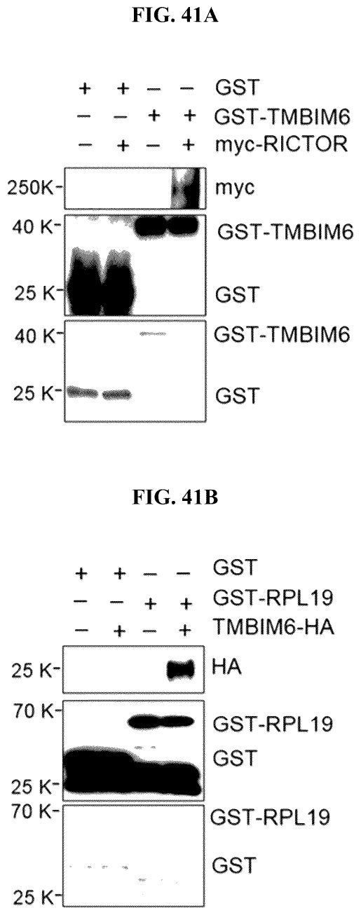

BI-1 was shown to bind BI-1 directly to RICTOR and RPL19 by glutathione S-transferase (GST) pull-down analysis associated with RPL19 and RIC TOR (see FIGS. 41A and 41B).

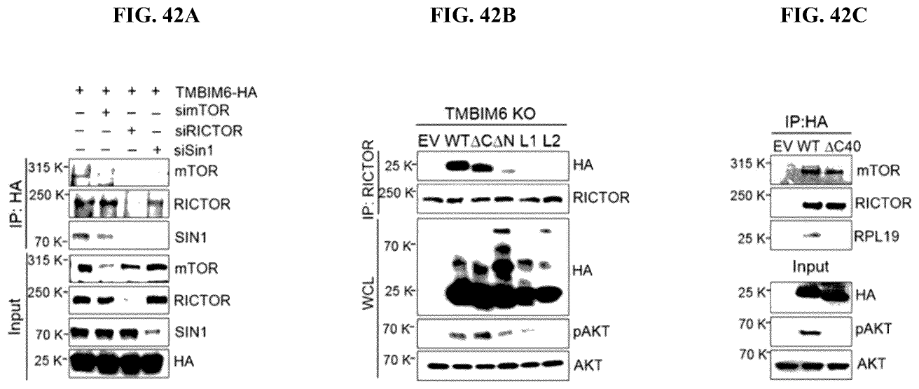

Immunoprecipitation with anti-RICTOR antibodies was performed to determine if BI-1 was related to mTORC2, and then the binding protein was analyzed using liquid chromatography-tandem mass spectrometry (LC-MS/MS). The results showed that BI-1 is one of the binding partners of mTORC2. In addition, we investigated the interaction between BI-1 (TMBIM6) and mTORC2 components. RCTOR is close to the FKBP12-rapamycin binding domain of mTOR and is bound by SIN1, while the mTOR kinase domain is bound by mLST840. RICTOR silencing by siRNA changes the interaction between mTORC2 and BI-1-HA mTOR dissociation was not observed (see FIG. 42A).

Deletion of 29 amino acids (AA) of the N-terminus (Δ N) and 9AA of the C-terminal (ΔC) to determine which BI-1 (TMBIM6) domain interacts with RIC TOR, and changes all residues of the cytoplasmic loop Containing BI-1 (TMBIM6) created mutant structures.

Loops 1 (L1) and 2 (L2) are connected by alanine residues for all six or seven transmembrane structures. The association between BI-1 and RICTOR was either reduced in BI-1-Δ N or was almost blocked by co-IP analysis at BI-1-L1 and L2 (see FIG. 42B).

To further study which BI-1 (TMBIM6) domain interacts with RPL19, we created a BI-1 (TMBIM6) mutation in which 40AA of the C-terminus (ΔC40) was lost. The immunoprecipitation assay confirmed that the RPL19 and BI-1-Δ C40 bindings were lost , while the interaction with RICTOR or mTOR remained unchanged (see FIG. 42C). Phosphorylation of AKT (pAKT-5473) was also reduced in RPL19 or RICTOR unassociated BI-1 (TMBIM6) mutants (see FIGS. 42B and 42C). These results indicate that BI-1 interacts with mTORC2 and ribosomes, and that this interaction is important for kinase activity of mTORC2.

### <Example 10> Topology of BI-1

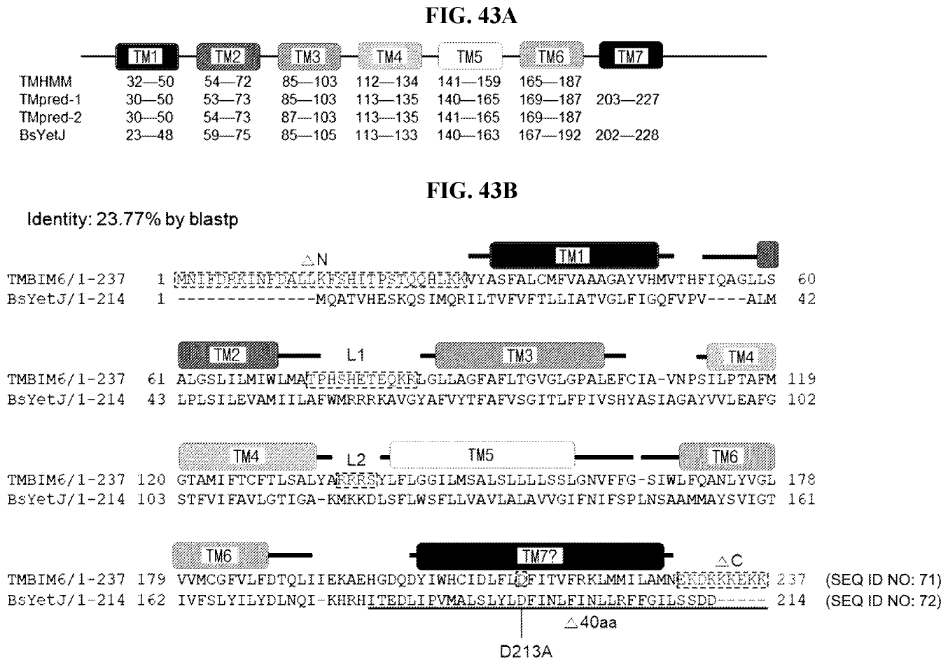

BI-1 (SEQ ID NO: 71) consists mostly of 6 or 7 transmembrane regions with an a-helix structure, and the C-terminus of BI-1 is present in the cytoplasm by TMHMM or in the ER intraductal space by bacterial homologue BsYetJ41-45 (See FIG. 43A). B sYetJ is a bacterial protein (SEQ ID NO: 72) associated with hBI-1, but amino acid identity by BLASTp is only 23.77% (see FIG. 43B).

To understand BI-1 (TMBIM6) topology, immunofluorescence staining was performed using cells overexpressing BI-1 with N-terminal (HA-BI-1) and C-terminal (BI-1-HA) HA tags. Triton X-100 permeates all membranes and induces staining of lumen and cytoplasmic epitopes, while digitonin has the property of only accessing the cytoplasmic epitopes of antibodies. PDI maintained in ER lumen was used as a negative control.

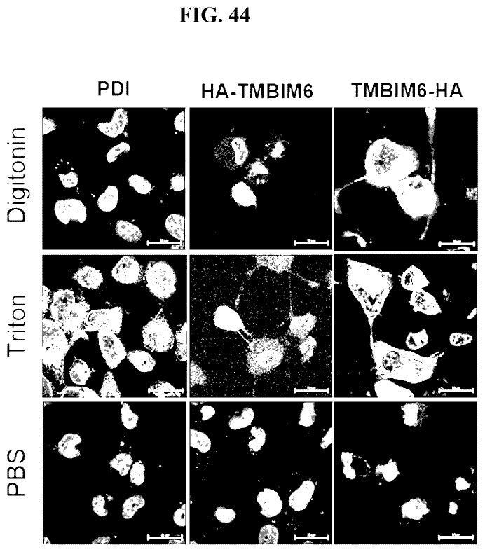

Considering that immunofluorescence of HA-BI-1 and BI-1-HA was detected under all conditions, but PDI fluorescence was not detected in the presence of digitonin (see FIG. 44), this result shows that the N- and C-terminus of BI-1 is 6- It suggests exposure to cytoplasm under transmembrane structure conditions.

### Example <11> Identification of Proteins Interacting With BI-1

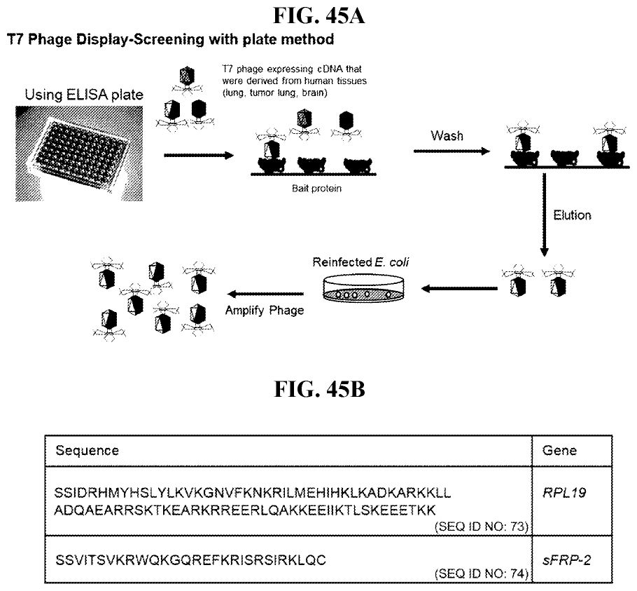

T4 phage display screening was performed using the cDNA library of human tissue and the 50 amino acid cytoplasmic domains of BI-1 as bait. 60S RPL19 (SEQ ID NO: 73) was found to act as a ligand for BI-1 (see FIGS. 45A and 45B). Consistent with the foregoing results, it suggests that physical interaction between BI-1 and RICOTR or ribosomes is necessary to enhance mTORC2 activity.

### Example <12> mTORC2 Activation Control by BI-1 (TMBIM6)-Associated ER Ca²⁺ Emission

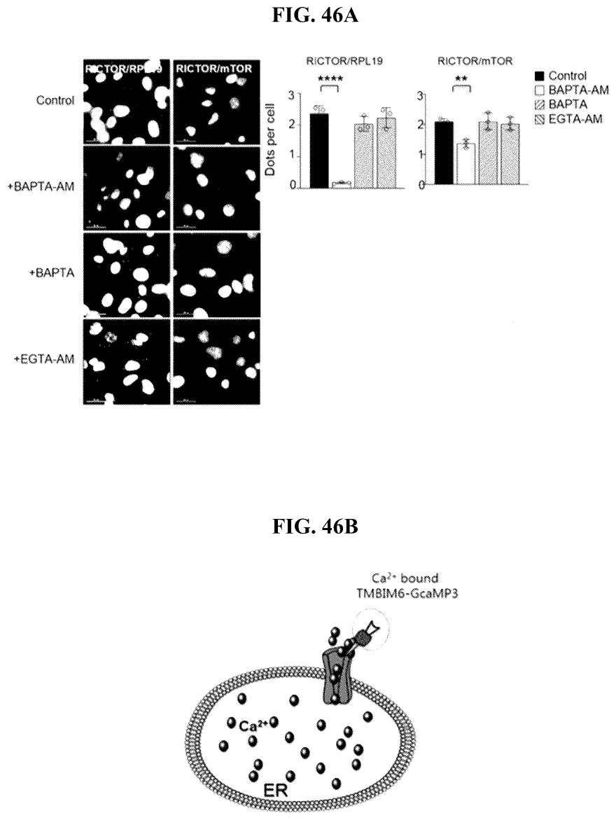

The role of Ca²⁺ in mTORC2 activation was investigated. Ca²⁺ depletion by BAPTA acetoxymethyl ester (BAPTA-AM) rather than BAPTA or slow Ca²⁺ chelator (ie, EGTA-AM) blocked the binding between mTORC2 and ribosomes as shown in PLA analysis results. (See FIG. 46A).

Thus, it was found that the topical Ca²⁺ concentration affected mTORC2 activation.

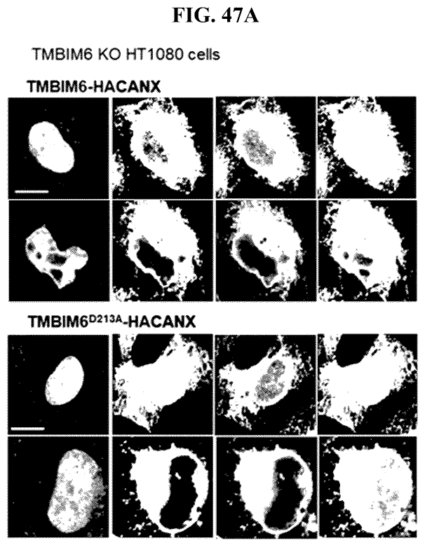

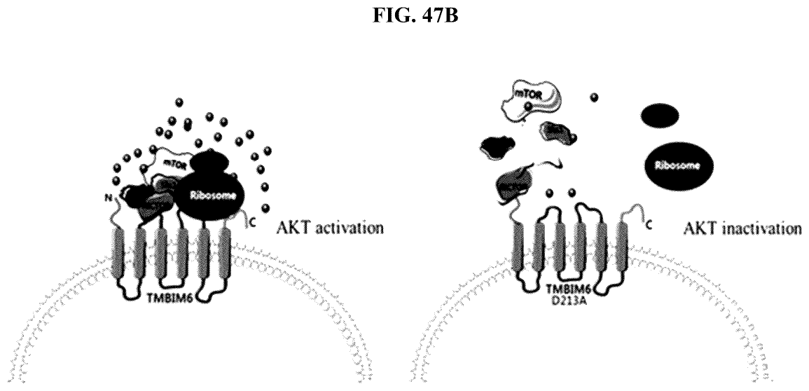

To evaluate whether the Ca²⁺ released from BI-1 affects the interaction between mTORC2 components and ribosomes, BI-1-GCaMP3 cells were first used to determine the Ca²⁺ leakage characteristics of BI-1. In HT213 cells to determine whether the calcium channel site D213A mutant cells and WT (WT) cell conditions (see FIG. 46B) affect mTORC2 assembly and AKT phosphorylation, first in HT1080 cells Expression patterns were identified (see FIG. 47A).

As a result of the confirmation, it was found that the expression degree and pattern in ER were maintained at a similar level in WT and BI-1D213A cells. Therefore, BI-1 (TMBIM6)-related AKT activation is characterized by protein interactions as follows. That is, it can be seen that the binding of RICTOR and BI-1 is independent of Ca²⁺ leakage, while the interaction of BI-1 with mTOR or ribosome depends on local Ca²⁺ leakage through BI- (see FIG. 47B).

### Example <13> Effect on mTORC2 Assembly and mTORC2 and Ribosome Association by BI-1-Induced Ca²⁺ Leakage

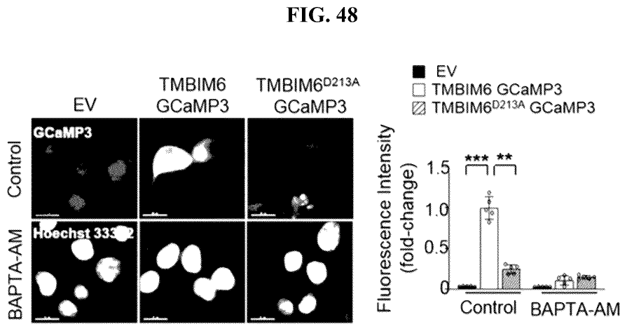

To evaluate whether the Ca²⁺ emitted from BI-1 affects the interaction between mTORC2 components and ribosomes, we first used BI-1-GCaMP3 to determine the Ca²⁺ leakage characteristics of BI-1. Ca²⁺ release associated with BI-1 (TMBIM6) shown by fluorescence intensity was detected in HT1080 cells expressing WT BI-1-GCaMP3, but not in Ca²⁺ channel mutant BI-1 (BI-1D213A)-GCaMP3 cells (see FIG. 48).

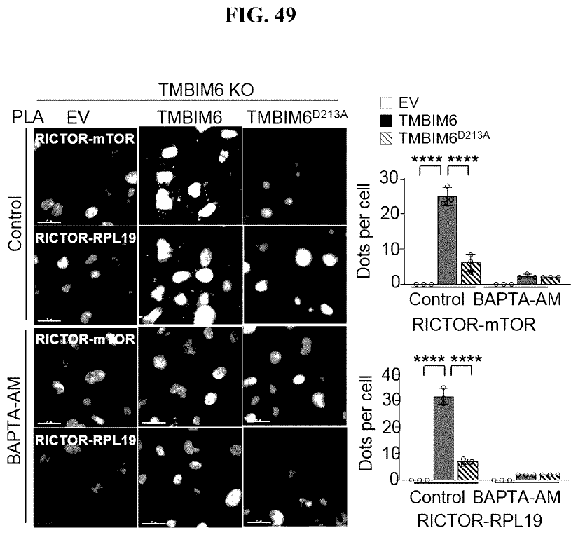

Next, PLA analysis is performed by RICTOR and mTOR; OR BETWEEN RICTOR AND RPL119; It was shown that the interaction was increased in HT1080 cells expressing BI-1 (TMBIM6) WT rather than BI-1D213A cells (see FIG. 49).

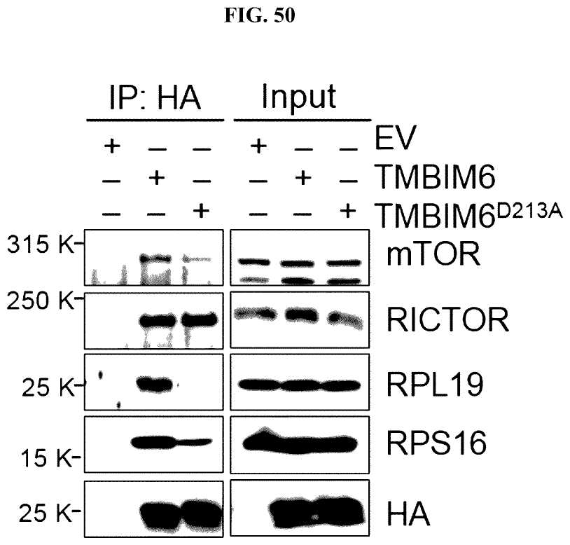

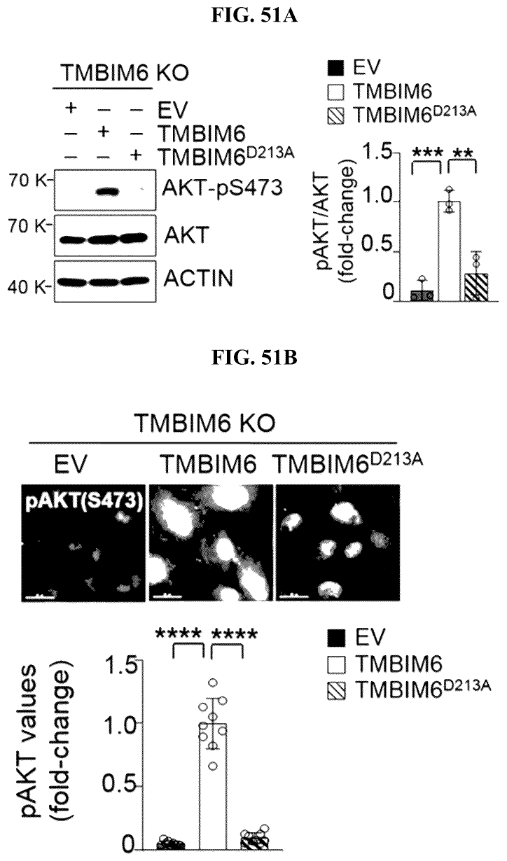

In co-IP analysis, BI-1 and RICTOR binding did not differ significantly between cells expressing WT and BI-1D213A. However, mTOR binding to BI-1 was slightly reduced in BI-1D213A cells. It is interesting to note that RPL19 and RPS16 binding to BI-1 was significantly reduced in D213A mutant cells (see FIG. 50). In addition, immunoblot and immunofluorescence analysis showed that phosphorylation of AKT in BI-1D213A cells was reduced (see FIGS. 51A and 51B).

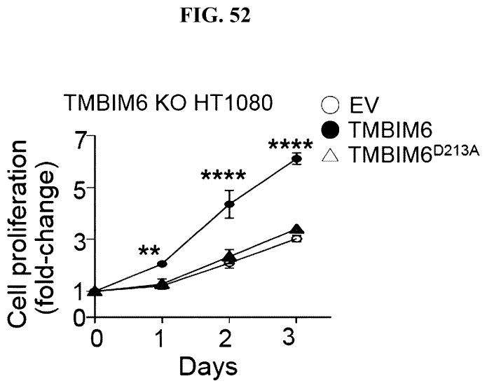

To investigate the importance of Ca²⁺ leakage through BI-1, we wanted to confirm the effect on cell metabolism after stably expressing WT (WT) or BI-1 (TMBIM6) D213A in BI-1 (TMBIM6) knockout HT1080 cells. Cell proliferation was recovered under BI-1 (TMBIM6) re-expression conditions in knockout cells regardless of the degree of BI-1 (TMBIM6) expression, and had little effect on cell proliferation in D213A re-expressed knockout cells (see FIG. 52).

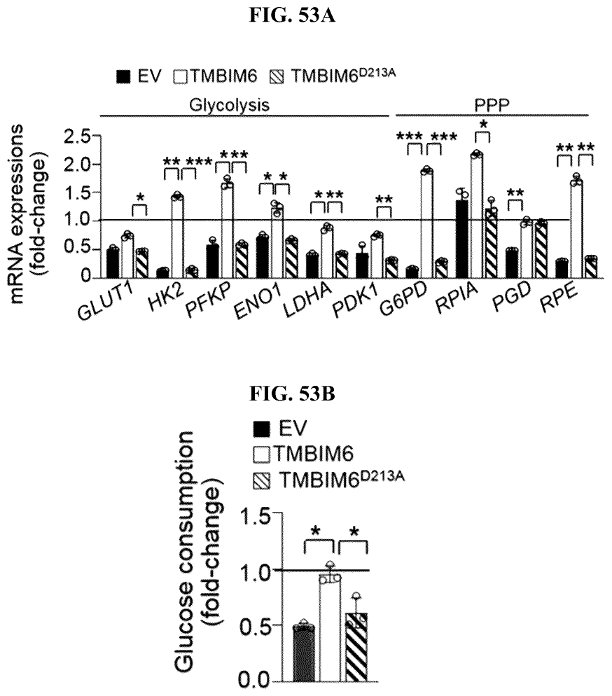

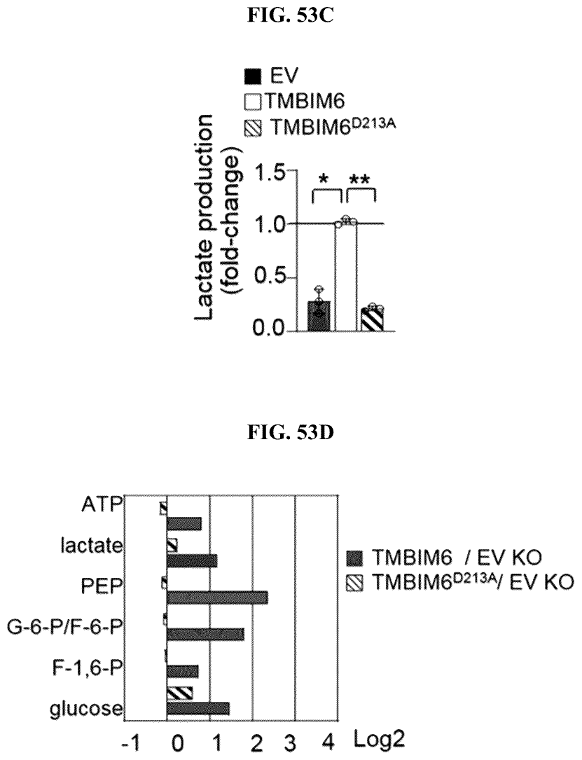

In addition, glycolysis and expression of genes associated with PPP showed recovery of glucose consumption and lactate production in cells that re-expressed BI-1 in BI-1 (TMBIM6) knockout cells (see FIGS. 53A-53D). By mass spectrometry, metabolite levels of glycolysis, TCA, PPP, and HBP were recovered in BI-1 (TMBIM6) structural cells compared to BI-1D213A cells (see FIG. 53D). These results suggest that BI-1 regulates the metabolic pathway through the “Ca²⁺ leakage-related mTORC2 activation” characteristic.

### Example <14> mTORC2-Dependent Metabolic Regulation Function by BI-1

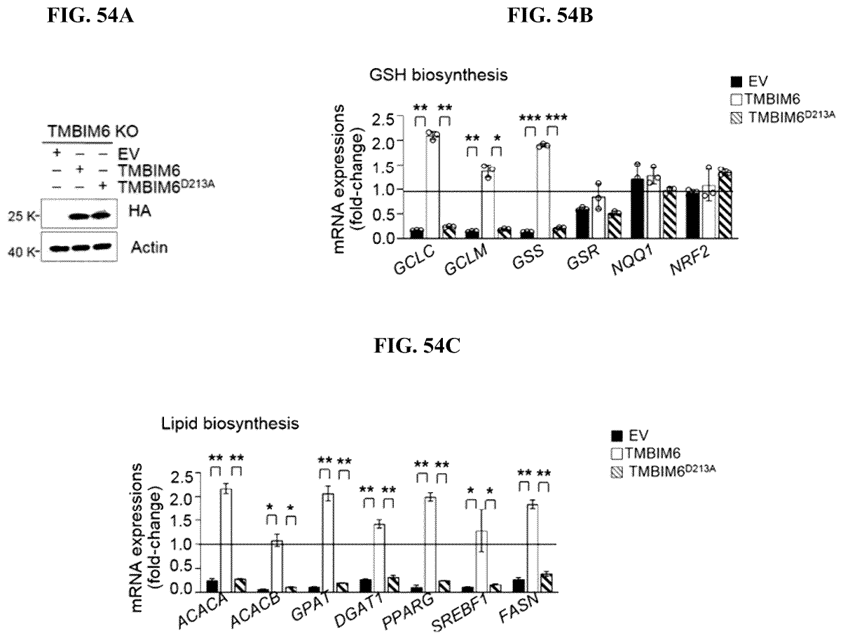

Expression of genes associated with GSH biosynthesis and de novo lipogenesis was recovered in BI-1 (TMBIM6) reexpressed knockout cells and did not affect D213A cells (see FIGS. 54A-54C).

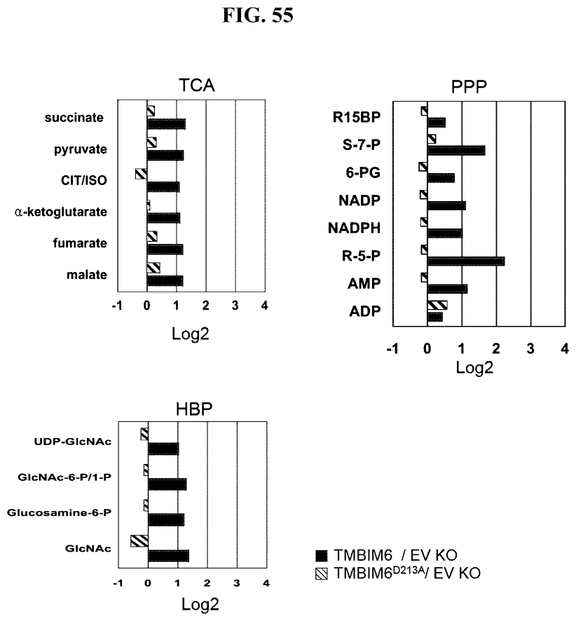

Mass spectrometry showed that metabolite levels of glycolysis, TCA, PPP and HBP were recovered in BI-1 (TMBIM6) structural cells compared to BI-1D213A cells (see FIG. 55).

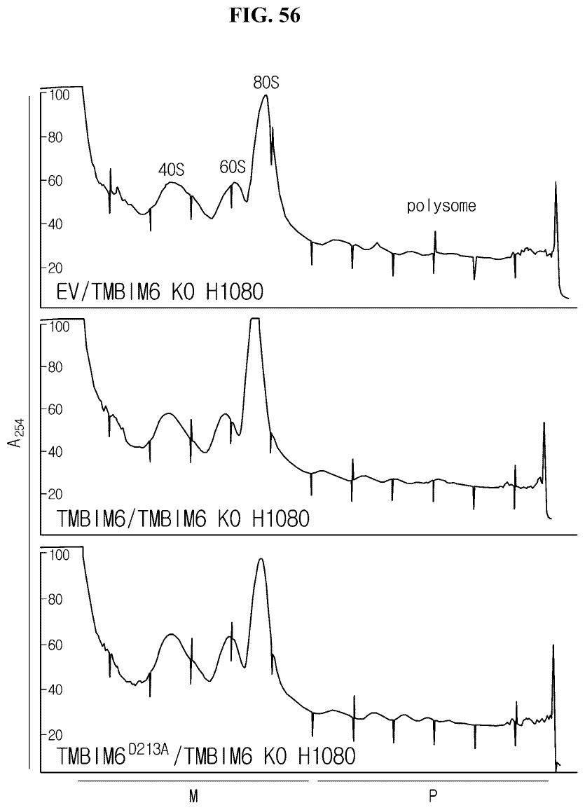

In addition, the pattern of ribosomal profiling was the same in all cells (see FIG. 56), indicating that BI-1 is independent of ribosomal maturation.

These results suggest that BI-1 regulates the metabolic pathway through its “Ca²⁺ leakage-related mTORC2 activation” characteristic.

### Example <15> Preparation of BI-1 (TMBIM6) Antagonist Compounds

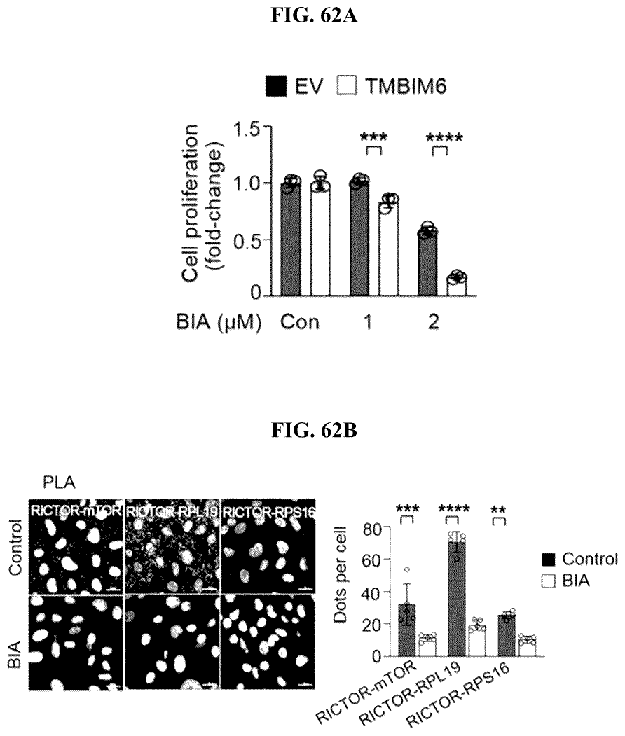

ER-TMBIM6-GCaMP3L1, a tool for detecting calcium glass from ER due to BI-1 (TMBIM6) shown in FIGS. 62A and 62B to find BI-1 (TMBIM6) antagonist compounds and ER—Screening by the Cepia assay method resulted in the following chalcone scaffold as a potential BI-1 (TMBIM6) antagonist.

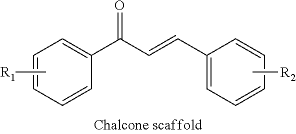

Various compounds were synthesized through the optimization of R1 and R2 positions with various substituents.

It was confirmed that 2E-1-2-aminophenyl-3-3-nitrophenyl-2-propen-1-one had an excellent antagonistic effect on BI-1 and named it BIA (BI Antagonist). In addition, analogs of BIA were synthesized, and the synthesized compounds were named as shown in the table below.

GM-90128

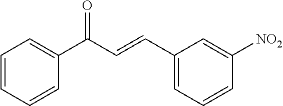 [SMILES: O=C(/C=C/c1cccc([N+](=O)[O-])c1)c1ccccc1]

GM-90129

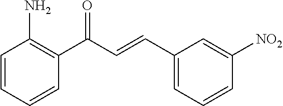 [SMILES: Nc1ccccc1C(=O)/C=C/c1cccc([N+](=O)[O-])c1]

GM-90130

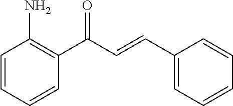 [SMILES: Nc1ccccc1C(=O)/C=C/c1ccccc1]

GM-90131

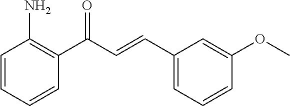 [SMILES: COc1cccc(/C=C/C(=O)c2ccccc2N)c1]

GM-90132

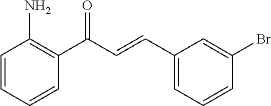 [SMILES: Nc1ccccc1C(=O)/C=C/c1cccc(Br)c1]

GM-90133

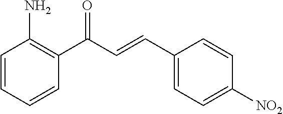 [SMILES: Nc1ccccc1C(=O)/C=C/c1ccc([N+](=O)[O-])cc1]

GM-90134

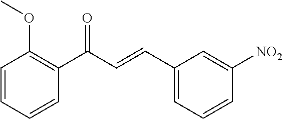 [SMILES: COc1ccccc1C(=O)/C=C/c1cccc([N+](=O)[O-])c1]

GM-90135

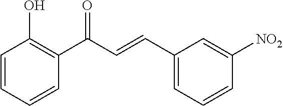 [SMILES: O=C(/C=C/c1cccc([N+](=O)[O-])c1)c1ccccc1O]

GM-90222

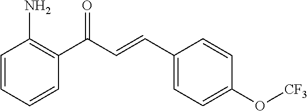 [SMILES: Nc1ccccc1C(=O)/C=C/c1ccc(OC(F)(F)F)cc1]

GM-90223

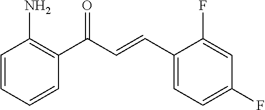 [SMILES: Nc1ccccc1C(=O)/C=C/c1ccc(F)cc1F]

GM-90224

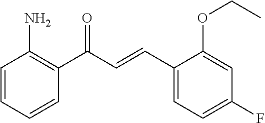 [SMILES: CCOc1cc(F)ccc1/C=C/C(=O)c1ccccc1N]

GM-90225

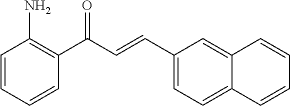 [SMILES: Nc1ccccc1C(=O)/C=C/c1ccc2ccccc2c1]

GM-90226

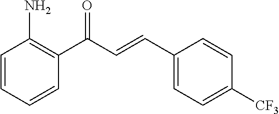 [SMILES: Nc1ccccc1C(=O)/C=C/c1ccc(C(F)(F)F)cc1]

GM-90227

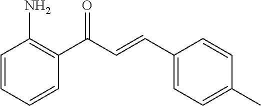 [SMILES: Cc1ccc(/C=C/C(=O)c2ccccc2N)cc1]

GM-90228

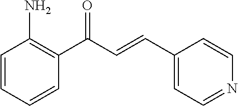 [SMILES: Nc1ccccc1C(=O)/C=C/c1ccncc1]

GM-90229

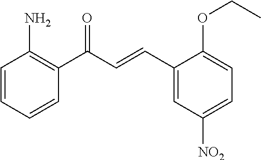 [SMILES: CCOc1ccc([N+](=O)[O-])cc1/C=C/C(=O)c1ccccc1N]

GM-90230

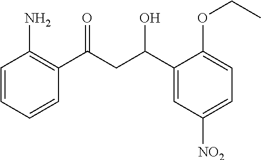 [SMILES: CCOc1ccc([N+](=O)[O-])cc1C(O)CC(=O)c1ccccc1N]

GM-90243

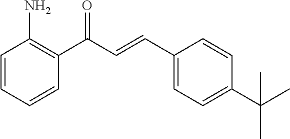 [SMILES: CC(C)(C)c1ccc(/C=C/C(=O)c2ccccc2N)cc1]

GM-90254

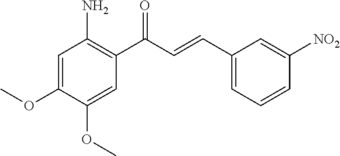 [SMILES: COc1cc(N)c(C(=O)/C=C/c2cccc([N+](=O)[O-])c2)cc1OC]

GM-90255

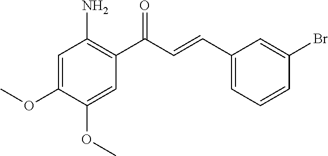 [SMILES: COc1cc(N)c(C(=O)/C=C/c2cccc(Br)c2)cc1OC]

GM-90256

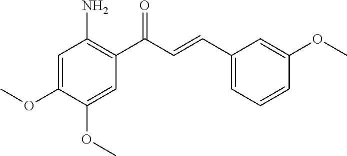

GM-90281

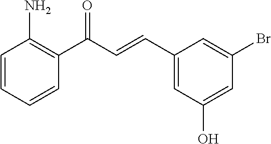 [SMILES: Nc1ccccc1C(=O)/C=C/c1cc(O)cc(Br)c1]

GM-90282

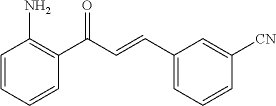 [SMILES: N#Cc1cccc(/C=C/C(=O)c2ccccc2N)c1]

GM-90283

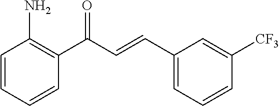 [SMILES: Nc1ccccc1C(=O)/C=C/c1cccc(C(F)(F)F)c1]

GM-90284

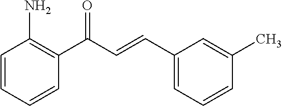 [SMILES: Cc1cccc(/C=C/C(=O)c2ccccc2N)c1]

GM-90285

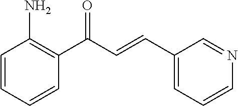 [SMILES: Nc1ccccc1C(=O)/C=C/c1cccnc1]

GM-90295

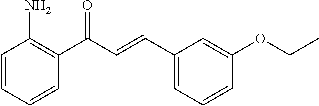 [SMILES: CCOc1cccc(/C=C/C(=O)c2ccccc2N)c1]

GM-90296

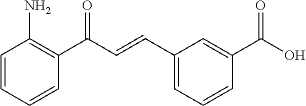 [SMILES: Nc1ccccc1C(=O)/C=C/c1cccc(C(=O)O)c1]

GM-90297

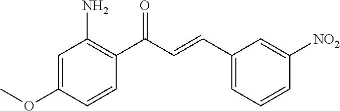 [SMILES: COc1ccc(C(=O)/C=C/c2cccc([N+](=O)[O-])c2)c(N)c1]

GM-90298

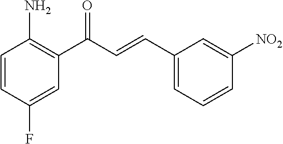 [SMILES: Nc1ccc(F)cc1C(=O)/C=C/c1cccc([N+](=O)[O-])c1]

GM-90299

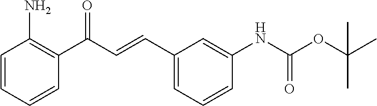 [SMILES: CC(C)(C)OC(=O)Nc1cccc(/C=C/C(=O)c2ccccc2N)c1]

GM-90300

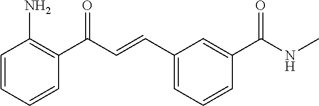 [SMILES: CNC(=O)c1cccc(/C=C/C(=O)c2ccccc2N)c1]

GM-90315

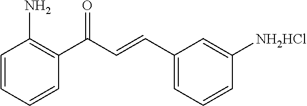

GM-90316

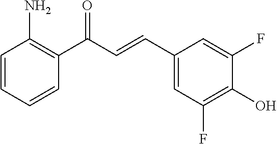 [SMILES: Nc1ccccc1C(=O)/C=C/c1cc(F)c(O)c(F)c1]

GM-90317

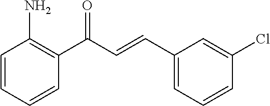 [SMILES: Nc1ccccc1C(=O)/C=C/c1cccc(Cl)c1]

GM-90318

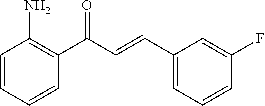 [SMILES: Nc1ccccc1C(=O)/C=C/c1cccc(F)c1]

GM-90319

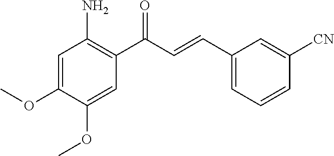 [SMILES: COc1cc(N)c(C(=O)/C=C/c2cccc(C#N)c2)cc1OC]

GM-90320

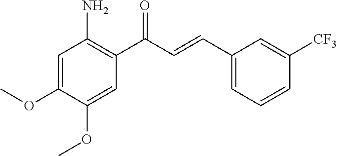 [SMILES: COc1cc(N)c(C(=O)/C=C/c2cccc(C(F)(F)F)c2)cc1OC]

GM-90321

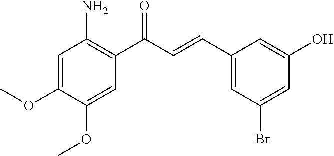 [SMILES: COc1cc(N)c(C(=O)/C=C/c2cc(O)cc(Br)c2)cc1OC]

GM-90337

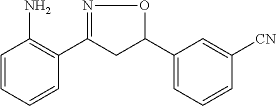 [SMILES: N#Cc1cccc(C2CC(c3ccccc3N)=NO2)c1]

GM-90338

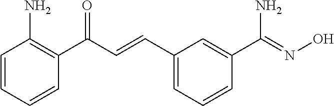 [SMILES: N/C(=N\O)c1cccc(/C=C/C(=O)c2ccccc2N)c1]

GM-90339

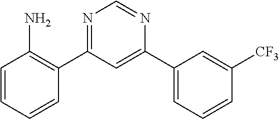 [SMILES: Nc1ccccc1-c1cc(-c2cccc(C(F)(F)F)c2)ncn1]

GM-90340

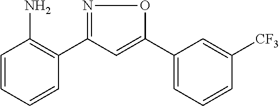 [SMILES: Nc1ccccc1-c1cc(-c2cccc(C(F)(F)F)c2)on1]

BIA

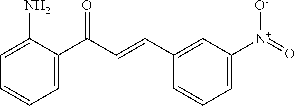 [SMILES: Nc1ccccc1C(=O)/C=C/c1cccc([N+](=O)[O-])c1]

Each compound was prepared in the following manner.

The derivative compounds of the present disclosure having the formula (1) were prepared as in the representative embodiments of the following semi condensation formula I, and are not limited thereto.

Among the compounds of formula 1 prepared as shown in Scheme I, GM-90222, GM-90223, GM-90224, GM-90229, GM-90230, GM-90243, GM-90254, GM-90255, GM-90259, GM-90230, GM-90315, GM-90316, GM-90319, GM-90320, GM-90321, GM-90337, GM-90338 , GM-90339, and GM-90340 are newly synthesized new compounds. Among the novel compounds, NMR data was described for GM-90223.

### Representative Examples

Substituted acetophenone (1.25 mmol) and sodium hydroxide (0.251 mmol) were dissolved in ethanol (5 mL), the mixture was stirred at room temperature for 10 minutes and then substituted benzaldehyde (1.272 mmol) was added . The reaction mixture was then stirred at room temperature and monitored with TLC using 25% ethyl acetate/nucleic acid as a solvent system. The solvent was evaporated and removed, the residue was treated with water (40 mL) and extracted with ethyl acetate (30 mL×3). The extracted and collected organic layer was dried and concentrated with sodium sulfate anhydrous and purified by silica gel column chromatography using a mixture of EA and Hexane to obtain the corresponding α,β-unsaturated ketone.

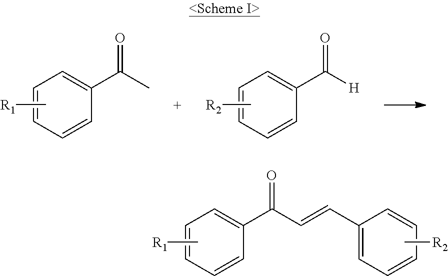

### Manufacturing Example 1. (E)-3-(3-nitrophenyl)-1-phenyl prop-2-en-1-one (GM-90128)

The title compound was prepared from acetophenone (120 mg, 1.00 mmol) and 3-nitrobenzaldehyde (154 mg, 1.02 mmol) according to representative embodiments above. 76% yield (192 mg, 0.76 mmol)

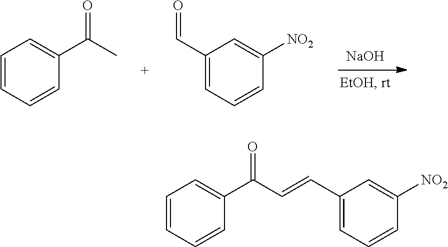

### Manufacturing Example 2. (E)-1-(2-aminophenyl)-3-(3-nitrophenyl) prop-2-en-1-one (GM-90129)

The heading compound was prepared from 1-(2-aminophenyl) ethane-1-one (135 mg, 1.00 mmol) and 3-nitrobenzaldehyde (154 mg, 1.02 mmol) according to representative embodiments above. 74% yield (199 mg, 0.74 mmol)

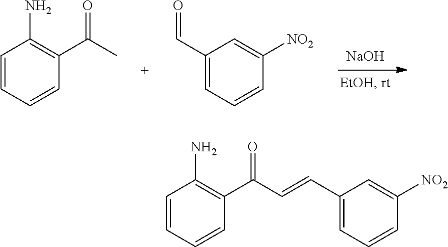

### Manufacturing Example 3. (E)-1-(2-aminophenyl)-3-phenylprop-2-en-1-one (GM-90130)

The title compound was prepared from 1-(2-aminophenyl) ethane-1-one (135 mg, 1.0 mmol) and benzaldehyde (108 mg, 1.02 mmol) according to representative embodiments above. 78% yield (174 mg, 0.78 mmol)

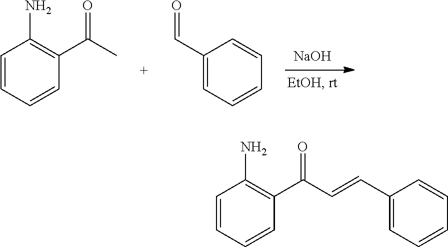

### Manufacturing Example 4. (E)-1-(2-aminophenyl)-3-(3-methoxyphenyl)prop-2-en-1-one (GM-90131)

The title compound was prepared from 1-(2-aminophenyl) ethane-1-one (135 mg, 1.00 mmol) and 3-methoxybenzaldehyde (139 mg, 1.02 mmol) according to representative embodiments above. 71% yield (180 mg, 0.71 mmol)

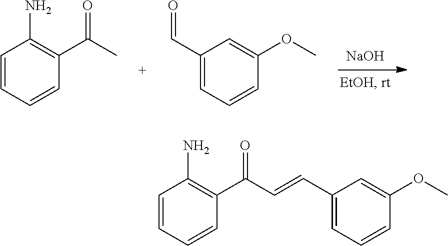

### Manufacturing Example 5. (E)-1-(2-aminophenyl)-3-(3-bromophenyl)prop-2-en-1-one (GM-90132)

The title compound was prepared from 1-(2-aminophenyl) ethane-1-one (135 mg, 1.00 mmol) and 3-bromobenzaldehyde (189 mg, 1.02 mmol) according to representative embodiments above. 82% yield (248 mg, 0.82 mmol)

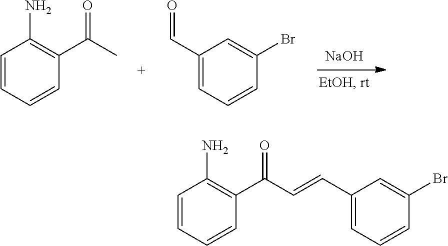

### Manufacturing Example 6. (E)-1-(2-aminophenyl)-3-(4-nitrophenyl)prop-2-en-1-one (GM-90133)

The title compound was prepared from 1-(2-aminophenyl) ethane-1-one (135 mg, 1.00 mmol) and 4-nitrobenzaldehyde (154 mg, 1.02 mmol) according to representative embodiments above. 80% yield (215 mg, 0.80 mmol)

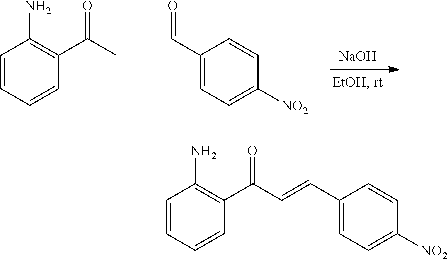

### Manufacturing Example 7. (E)-1-(2-methoxyphenyl)-3-(3-nitrophenyl)prop-2-en-1-one (GM-90134)

The title compound was prepared from 1-(2-methoxyphenyl) ethane-1-one (150 mg, 1.00 mmol) and 3-nitrobenzaldehyde (154 mg, 1.02 mmol) according to representative embodiments above. 80% yield (227 mg, 0.80 mmol)

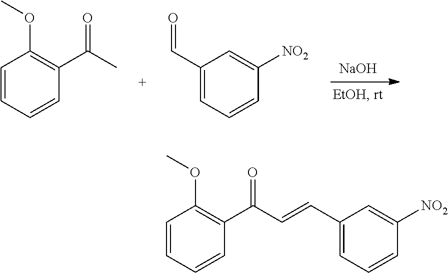

### Manufacturing Example 8. (E)-1-(2-hydroxyphenyl)-3-(3-nitrophenyl)prop-2-en-1-one (GM-90135)

1M BCl₃ (in dichloromethane, 0.3 mL) was added at 0° C. to the GM-90134 (57 mg, 0.20 mmol) solution in dichloromethane. The reaction mixture was stirred at 0° C. for 3 hours. After completion, the reaction mixture was treated with water (3 mL) and extracted with dichloromethane (5 mL×3). The organic layers were combined and dried and concentrated on sodium sulfate anhydrous and purified by silica gel column chromatography using a mixture of EA and hexane to obtain GM-90135 at a yield of 90% (48 mg, 0.18 mmol).

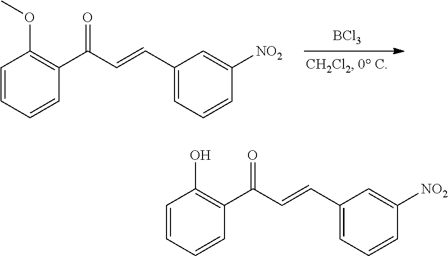

### Manufacturing Example 9. (E)-1-(2-aminophenyl)-3-(4-(trifluoromethoxy)phenyl)prop-2-en-1-one (GM-90222)

The title compound was prepared from 1-(2-aminophenyl) ethane-1-one (135 mg, 1.0 mmol) and 4-(trifluoromethoxy) benzaldehyde (194 mg, 1.02 mmol) according to representative embodiments above. 84% yield (258 mg, 0.84 mmol)

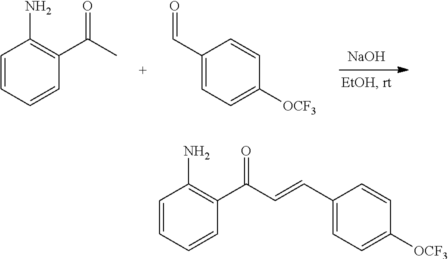

### Manufacturing Example 10. (E)-1-(2-aminophenyl)-3-(2,4-difluorophenyl)prop-2-en-1-one (GM-90223)

The title compound was prepared from 1-(2-aminophenyl) ethane-1-one (135 mg, 1.0 mmol) and 2,4-difluorbenzaldehyde (145 mg, 1.02 mmol) according to representative embodiments above. 83% yield (215 mg, 0.83 mmol)

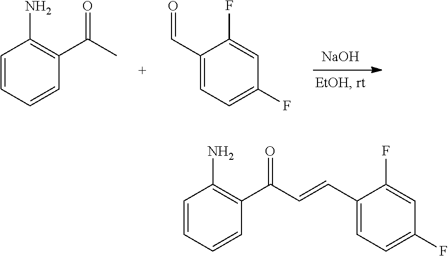

¹H NMR (500 MHz, CDCl₃) δ7.82 (dd, J=8.4, 1.5 Hz, 1H), 7.75 (d, J=15.8 Hz, 1H), 7.65 (d, J=15.7 Hz, 1H), 7.60 (td, J=8.5, 6.4 Hz, 1H), 7.28 (ddd, J=8.5, 7.2, 1.5 Hz, 1H), 6.94-6.89 (m, 1H), 6.87 (ddd, J=11.1, 8.8, 2.5 Hz, 1H), 6.69 (d, J=6.8 Hz, 1H), 6.67 (dt, J=5.4, 3.3 Hz, 1H).

### Manufacturing Example 11. (E)-1-(2-aminophenyl)-3-(2-ethoxy-4-fluorophenyl)prop-2-en-1-one (GM-90224)

The heading compound was prepared from 1-(2-aminophenyl) ethan-1-one (135 mg, 1.0 mmol) and 2,4-ethoxy-4-fluorobenzaldehyde (172 mg, 1.02 mmol) according to the usual procedure. 70% yield (200 mg, 0.70 mmol)

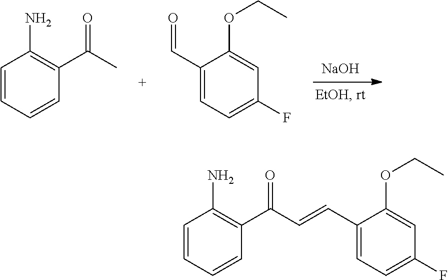

### Manufacturing Example 12. (E)-1-(2-aminophenyl)-3-(naphthalen-2-yl) prop-2-en-1-one (GM-90225)

The title compound was prepared from 1-(2-aminophenyl) ethane-1-one (135 mg, 1.0 mmol) and 2-naphthalaldehyde (159 mg, 1.02 mmol) according to representative embodiments above. 79% yield (216 mg, 0.79 mmol)

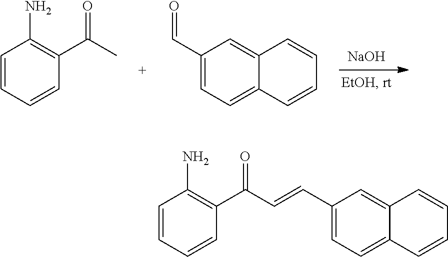

### Manufacturing Example 13. (E)-1-(2-aminophenyl)inophenyl)-3-(4-(trifluoromethyl)phenyl)prop-2-en-1-one (GM-90226)

The title compound was prepared from 1-(2-aminophenyl) ethane-1-one (135 mg, 1.0 mmol) and 4-(trifluoromethyl) benzaldehyde (178 mg, 1.02 mmol) according to the representative embodiment above. 85% yield (253 mg, 0.85 mmol)

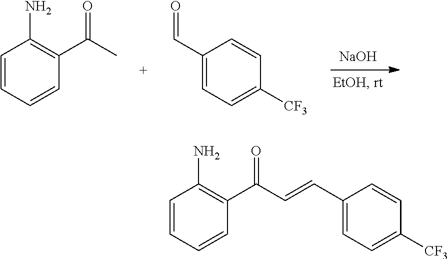

### Manufacturing Example 14. (E)-1-(2-aminophenyl)-3-(p-tolyl)prop-2-en-1-one (GM-90227)

The title compound is 1-(2-aminophenyl) ethane-1-one (135 mg, 1.00 mmol) and 4-methylbenzaldehyde (123 mg, 1.02 mmol) It was prepared from. 77% yield (183 mg, 0.77 mmol)

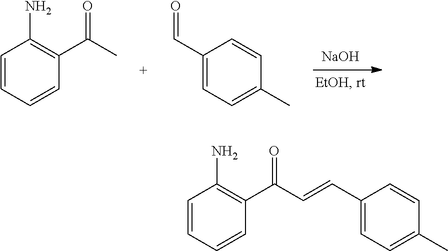

### Manufacturing Example 15. (E)-1-(2-aminophenyl)-3-(pyridin-4-yl)prop-2-en-1-one (GM-90228)

The title compound was prepared from 1-(2-aminophenyl) ethane-1-one (135 mg, 1.0 mmol) and isonicotinaldehyde (109 mg, 1.02 mmol) according to representative embodiments above. 69% yield (155 mg, 0.69 mmol)

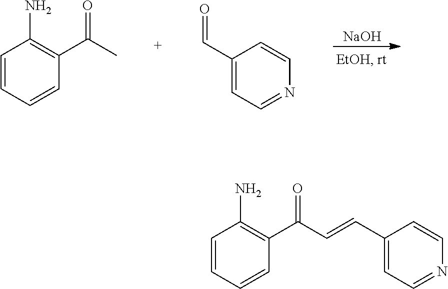

### Manufacturing Example 16. (E)-1-(2-aminophenyl)-3-(2-ethoxy-5-nitrophenyl)prop-2-en-1-one (GM-90229)

The title compound was prepared from 1-(2-aminophenyl) ethane-1-one (135 mg, 1.0 mmol) and 2-ethoxy-5-nitrobenzaldehyde (199 mg, 1.02 mmol) according to the representative embodiment above. 52% yield (162 mg, 0.52 mmol)

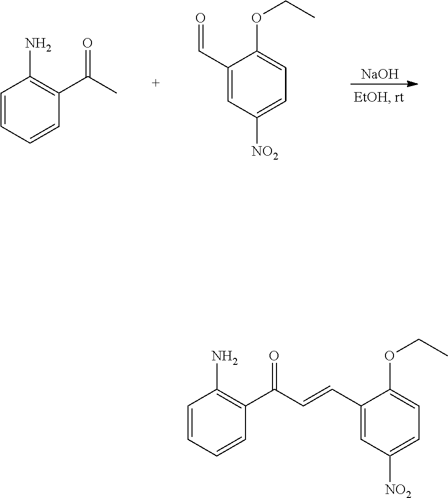

### Manufacturing Example 17. 1-(2-aminophenyl)-3-(2-ethoxy-5-nitro phenyl)-3-hydroxypropan-1-one (GM-90230)

The title compound was prepared from 1-(2-aminophenyl) ethane-1-one (135 mg, 1.00 mmol) and 2-ethoxy-5-nitrobenzaldehyde (199 mg, 1.02 mmol) according to the representative embodiment above. 34% yield (112 mg, 0.34 mmol)

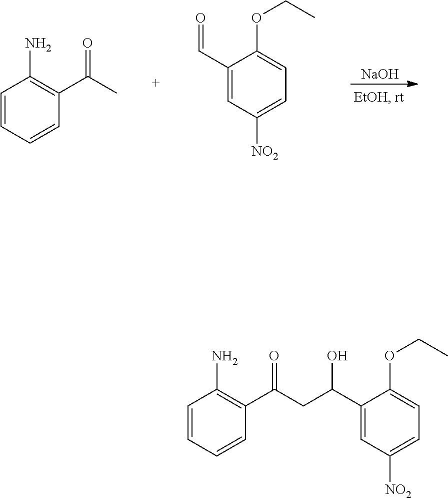

### Manufacturing Example 18. (E)-1-(2-aminophenyl)-3-(4-(tert-butyl)phenyl)prop-2-en-1-one (GM-90243)

The title compound was prepared from 1-(2-aminophenyl) ethane-1-one (135 mg, 1.0 mmol) and 4-(tert-butyl) benzaldehyde (165 mg, 1.02 mmol) according to representative embodiments above. 81% yield (226 mg, 0.81mmol)

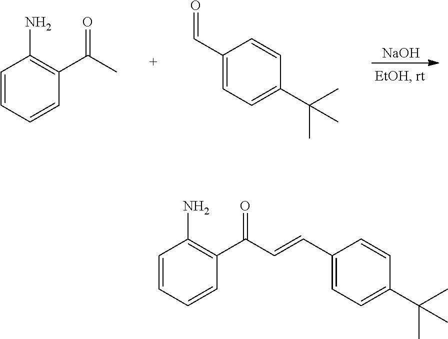

### Manufacturing Example 19. (E)-1-(2-amino-4,5-dimethoxyphenyl)-3-(3-nitrophenyl)prop-2-en-1-one (GM-90254)

The title compound was prepared from 1-(2-amino-4,5-dimethoxyphenyl) ethane-1-one (195 mg, 1.0 mmol) and 3-nitrobenzaldehyde (154 mg, 1.02 mmol) according to the representative embodiment above. 84% yield (276 mg, 0.84 mmol)

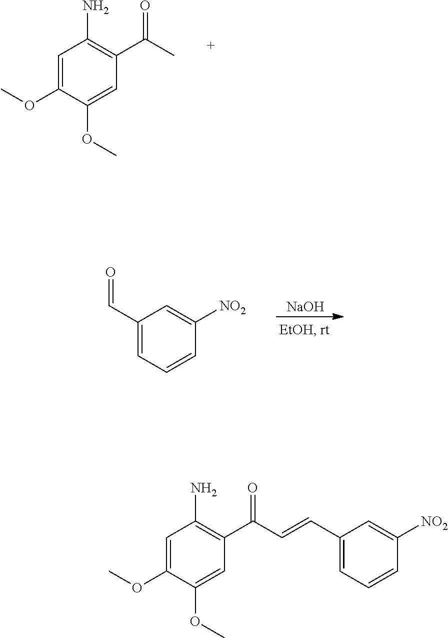 [SMILES: COc1cc(N)c(C(C)=O)cc1OC ; O=Cc1cccc([N+](=O)[O-])c1 ; "NaOH" ; COc1cc(N)c(C(=O)/C=C/c2cccc([N+](=O)[O-])c2)cc1OC]

### Manufacturing Example 20. (E)-1-(2-amino-4,5-dimethoxyphenyl)-3-(3-bromophenyl)prop-2-en-1-one (GM-90255)

The title compound was prepared from 1-(2-amino-4,5-dimethoxyphenyl) ethane-1-one (195 mg, 1.0 mmol) and 3-bromobenzaldehyde (188 mg, 1.02 mmol) according to the representative embodiment above. 80% yield (290 mg, 0.80 mmol)

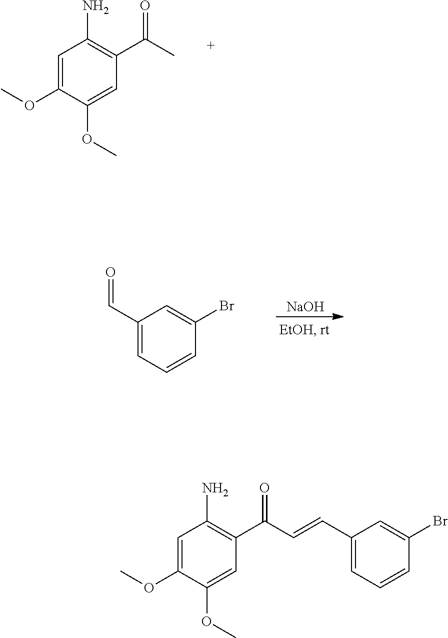 [SMILES: COc1cc(N)c(C(C)=O)cc1OC ; O=Cc1cccc(Br)c1 ; "NaOH" ; COc1cc(N)c(C(=O)/C=C/c2cccc(Br)c2)cc1OC]

### Manufacturing Example 21. (E)-1-(2-amino-4,5-dimethoxyphenyl)-3-(3-methoxyphenyl)prop-2-en-1-one (GM-90256)

The title compound was prepared from 1-(2-amino-4,5-dimethoxyphenyl) ethane-1-one (195 mg, 1.0 mmol) and 3-methoxybenzaldehyde (139 mg, 1.02 mmol) according to the representative embodiment above. 70% yield (219 mg, 0.70 mmol)

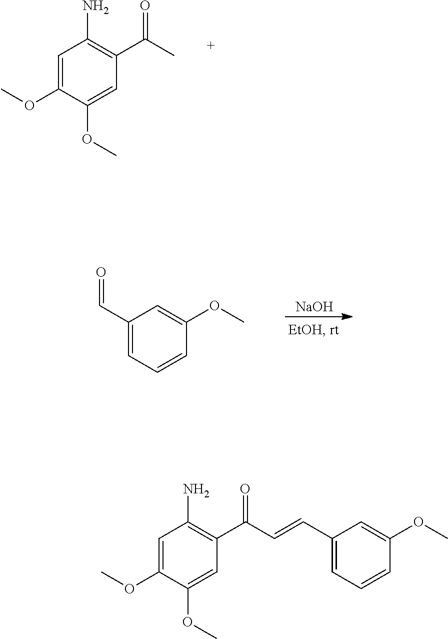

### Manufacturing Example 22. (E)-1-(2-aminophenyl)-3-(3-bromo-5-hydroxyphenyl)prop-2-en-1-one (GM-90281)

The title compound was prepared from 1-(2-aminophenyl) ethane-1-one (135 mg, 1.0 mmol) and 3-bromo-5-hydroxybenzaldehyde (205 mG, 1.02 mmol) according to representative embodiments above. 61% yield (194 mg, 0.61mmol)

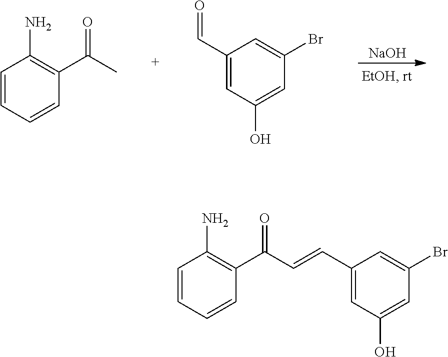

### Manufacturing Example 23. (E)-3-(3-(2-aminophenyl)-3-oxoprop-1-and-1-yl)benzonitrile (GM-90282)

The title compound was prepared from 1-(2-aminophenyl) ethane-1-one (135 mg, 1.00 mmol) and 3-formylbenzonitrile (134 mg, 1.02 mmol) according to representative embodiments described above. 77% yield (191 mg, 0.77 mmol)

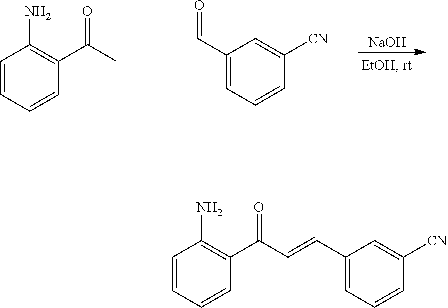

### Manufacturing Example 24. (E)-1-(2-aminophenyl)-3-(3-(trifluoromethyl)phenyl)prop-2-en-1-one (GM-90283)

The title compound was prepared from 1-(2-aminophenyl) ethane-1-one (135 mg, 1.0 mmol) and 3-(trifluoromethyl) benzaldehyde (178 mg, 1.02 mmol) according to the representative embodiment above. 77% yield (224 mg, 0.77 mmol)

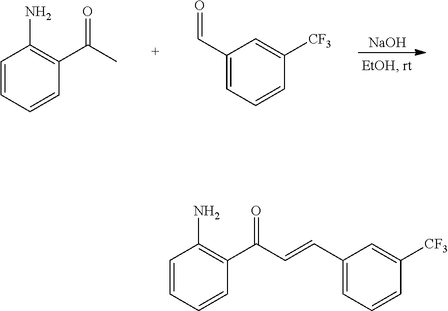

### Manufacturing Example 25. (E)-1-(2-aminophenyl)-3-(m-tolyl)prop-2-en-1-one (GM-90284)

The title compound was prepared from 1-(2-aminophenyl) ethane-1-one (135 mg, 1.0 mmol) and 3-methylbenzaldehyde (123 mg, 1.02 mmol) according to representative embodiments above. 68% yield (161 mg, 0.68 mmol)

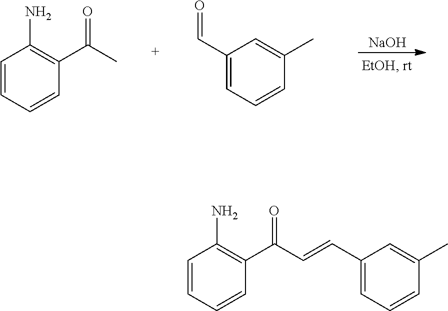

### Manufacturing Example 26. (E)-1-(2-aminophenyl)-3-(pyridin-3-yl)prop-2-en-1-one (GM-90285)

The title compound was prepared from 1-(2-aminophenyl) ethane-1-one (135 mg, 1.00 mmol) and nicotinaldehyde (109 mg, 1.02 mmol) according to representative embodiments above. 70% yield (157 mg, 0.70 mmol)

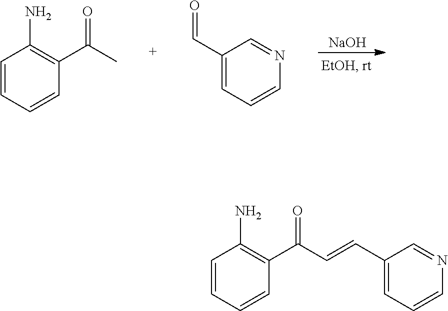

### Manufacturing Example 27. (E)-1-(2-aminophenyl)-3-(3-ethoxyphenyl)prop-2-en-1-one (GM-90295)

The title compound was prepared from 1-(2-aminophenyl) ethane-1-one (135 mg, 1.0 m mol) and 3-ethoxybenzaldehyde (153 mg, 1.02 mmol) according to the representative embodiment above. 71% yield (190 mg, 0.71mmol)

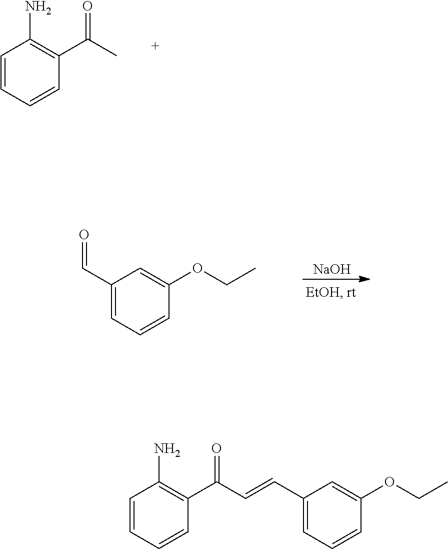

### Manufacturing Example 28. (E)-3-(3-(2-aminophenyl)-3-oxoprop-1-en-1-yl)benzoic Acid (GM-90296)

The title compound was prepared from 1-(2-aminophenyl) ethane-1-one (135 mg, 1.0 mmol) and 3-formylbenzoic acid (123 mg, 1.02 mmol) according to the representative embodiment above. 67% yield (179 mg, 0.67 mmol)

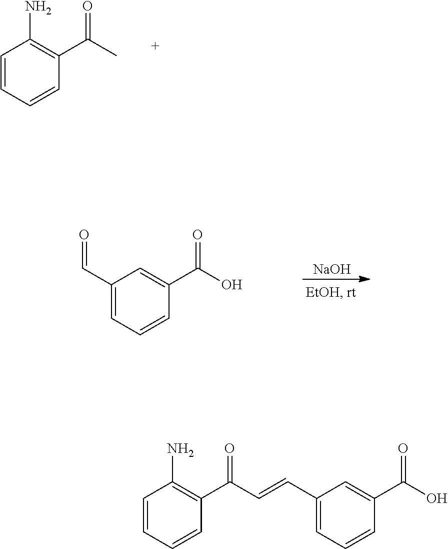

### Manufacturing Example 29. (E)-1-(2-amino-4-methoxyphenyl)-3-(3-nitrophenyl)prop-2-en-1-one (GM-90297)

The title compound was prepared from 1-(2-amino-4-methoxyphenyl) ethane-1-one (165 mg, 1.00 mmol) and 3-nitrobenzaldehyde (154 mg, 1.02 mmol) according to the representative embodiment above. 85% yield (254 mg, 0.85 mmol)

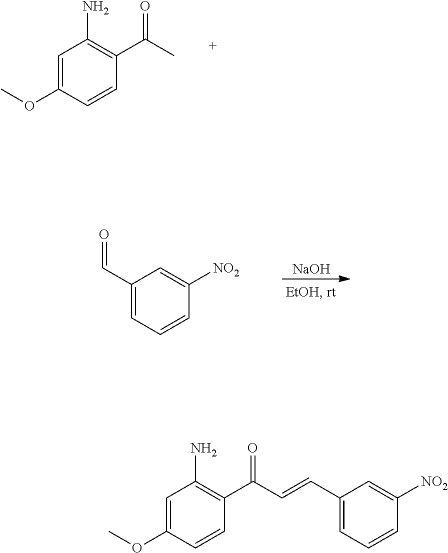 [SMILES: COc1ccc(C(C)=O)c(N)c1 ; O=Cc1cccc([N+](=O)[O-])c1 ; "NaOH" ; COc1ccc(C(=O)/C=C/c2cccc([N+](=O)[O-])c2)c(N)c1]

### Manufacturing Example 30. (E)-1-(2-amino-5-fluorophenyl)-3-(3-nitrophenyl)prop-2-en-1-one (GM-90298)

The title compound was prepared from 1-(2-amino-5-fluorophenyl) ethane-1-one (153 mg, 1.0 mmol) and 3-nitrobenzaldehyde (154 mg, 1.02 mmol) according to representative embodiments above. 82% yield (235 mg, 0.82 mmol)

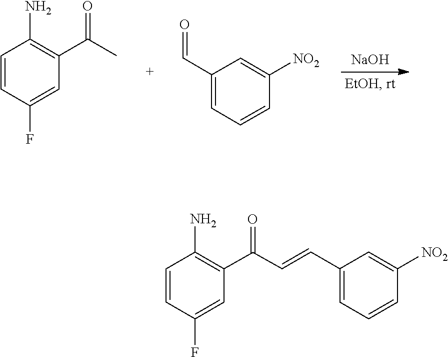

### Manufacturing Example 31. tert-butyl (E)-(3-(3-(2-aminophenyl)-3-oxoprop-1-en-1-yl)phenyl) carbamate (GM-90299)

The heading compound is 1-(2-aminophenyl) ethane-1- according to the representative embodiment described above.

It was prepared from one (135 mg, 1.0 mmol) and tert-butyl (3-formylphenyl) carbamate (226 mg, 1.02 mmol). 73% yield (247 mg, 0.73 mmol)

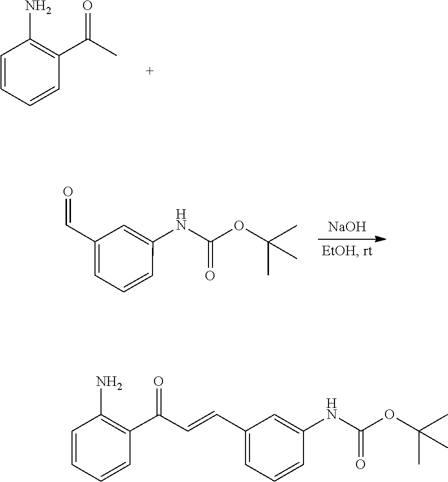

### Manufacturing Example 32. (E)-3-(3-(2-aminophenyl)-3-oxoprop-1-en-1-yl)-N-methylbenze Measured (GM-90300)

The title compound was prepared from 1-(2-amino-4-methoxyphenyl) ethane-1-one (165 mg, 1.00 mmol) and 3-formyl-N-methylbenzamide (166 mg, 1.02 mmol) according to representative embodiments above. 83% yield (233 mg, 0.83 mmol)

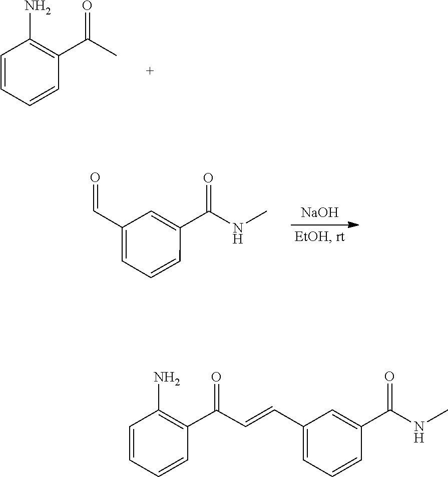

### Manufacturing Example 33. (E)-1-(2-aminophenyl)-3-(3-aminophenyl)prop-2-en-1-one Hydrochloride (GM-90315)

In a solution of GM-90299 (68 mg, 0.20 mmol) in dry dioxane (0.5 mL), 4M HC1 (in dioxane, 2 mL) was added. The reaction was stirred at room temperature for 3 hours. It was then concentrated and dried in a vacuum to obtain GM-90315 at a yield of 98% (54 mg, 0.20 mmol).

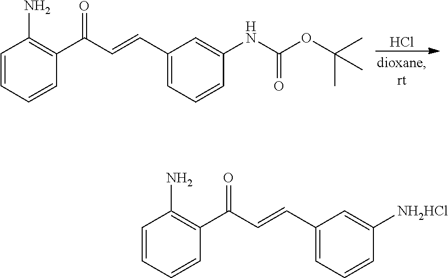

### Manufacturing Example 34. (E)-1-(2-aminophenyl)-3-(3,5-difluoro-4-hydroxyphenyl)prop-2-en-1-one (GM-90316)

The title compound was prepared from 1-(2-aminophenyl) ethane-1-one (135 mg, 1.0 mmol) and 3,5-difluoro-4-hydroxybenzaldehyde (161 mg, 1.02 mmol) according to representative embodiments above. 77% yield (212 mg, 0.77 mmol)

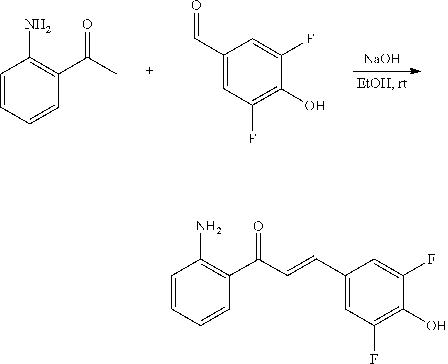

### Manufacturing Example 35. (E)-1-(2-aminophenyl)-3-(3-chlorophenyl)prop-2-en-1-one (GM-90317)

The title compound was prepared from 1-(2-aminophenyl) ethane-1-one (135 mg, 1.0 mmol) and 3-chlorobenzaldehyde (143 mg, 1.02 mmol) according to representative embodiments above. 85% yield (219 mg, 0.85 mmol)

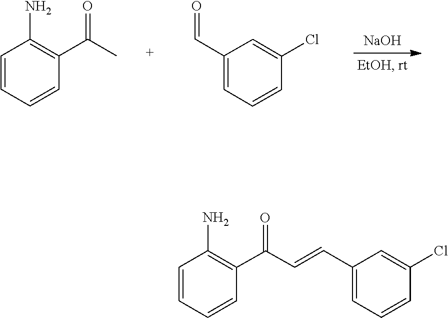

### Manufacturing Example 36. (E)-1-(2-aminophenyl)-3-(3-fluorophenyl)prop-2-en-1-one (GM-90318)

The title compound was prepared from 1-(2-aminophenyl) ethane-1-one (135 mg, 1.0 mmol) and 3-fluorobenzaldehyde (127 mg, 1.02 mmol) according to the representative embodiment above. 85% yield (205 mg, 0.85 mmol)

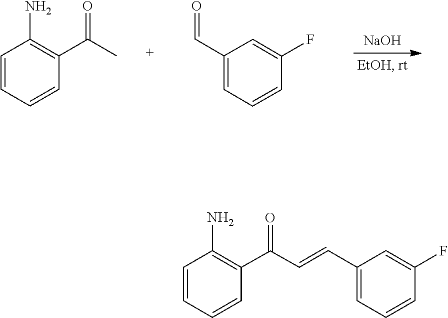

### Manufacturing Example 37. (E)-3-(3-(2-amino-4,5-dimethoxyphenyl)-3-oxoprop-1-en-1-yl) Benzonitrile (GM-90319)

The title compound was prepared from 1-(2-amino-4,5-dimethoxyphenyl) ethane-1-one (195 mg, 1.0 mmol) and 3-formylbenzonitrile (134 mg, 1.02 mmol) according to representative embodiments above. 80% yield (247 mg, 0.80 mmol)

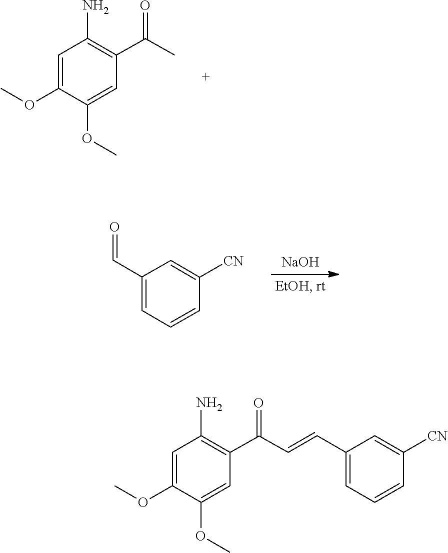

### Manufacturing Example 38. (E)-1-(2-amino-4,5-dimethoxyphenyl)-3-(3-(trifluoromethyl)phenyl)prop-2-en-1-one (GM-90320)

The title compound was prepared from 1-(2-amino-4,5-dimethoxyphenyl) ethane-1-one (195 mg, 1.0 mmol) and 3-(trifluoromethyl) benzaldehyde (178 mg, 1.02 mmol) according to the representative embodiment above. 82% yield (288 mg, 0.82 mmol)

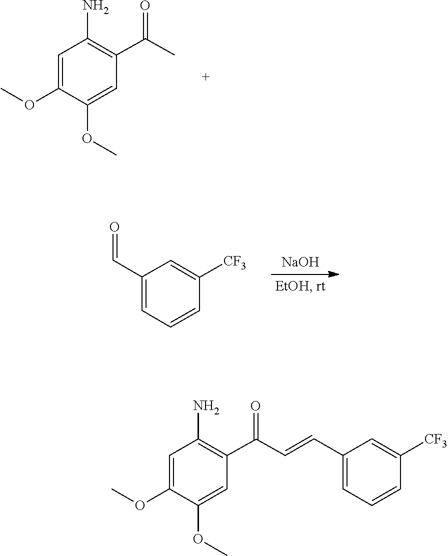

### Manufacturing Example 39. (E)-1-(2-amino-4,5-dimethoxyphenyl)-3-(3-bromo-5-hydroxyphenyl)prop-2-en-1-one (GM-90321)

The title compound was prepared from 1-(2-amino-4,5-dimethoxyphenyl) ethane-1-one (195 mg, 1.0 mmol) and 3-bromo-5-hydroxybenzaldehyde (205 mg, 1.02 mmol) according to representative embodiments above. 72% yield (272 mg, 0.72 mmol)

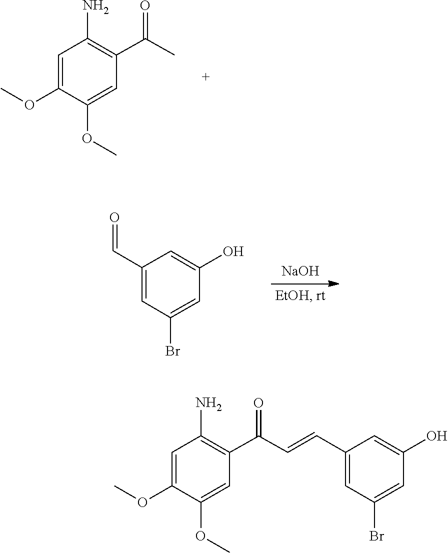

### Manufacturing Example 40. 3-(3-(2-aminophenyl)isoxazol-5-yl)benzonitrile (GM-90337)

K2 CO₃ (221 mg, 1.60 mmol) is added to a solution of N-hydroxyl-4-toluenesulfonamide (281 mg, 1.50 mmol) dissolved in 1.4 mL methanol/water (6: 1), followed by 0.2 mmol GM-90282 (50 mg) dissolved in 0.6 ml of methanol, Reaction The mixture was stirred at 40° C. for 24 hours and monitored with TLC. An additional K₂CO₃ (111 mg, 0.80 mmol) was then added and the mixture was stirred at 40° C. for 10 hours. After completion of stirring, the reaction mixture was treated with water (3 mL) and extracted with ethyl acetate (5 mL×3). The extracted organic layer was dried on anhydrous natrium sulfate, concentrated, and purified by silica gel column chromatography using a mixture of EA and hexane to obtain GM-90337 at a yield of 68% (36 mg, 0.136 mmol).

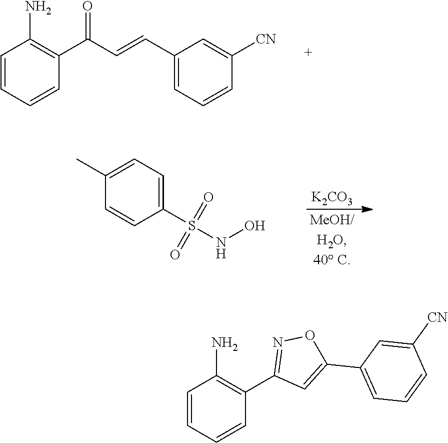

### Manufacturing Example 41. (Z)-3-((E)-3-(2-aminophenyl)-3-oxoprop-1-en-1-yl)-N′-hydroxy Benzimidamide (GM-90338)

The title compound was prepared from 1-(2-aminophenyl) ethane-1-one (135 mg, 1.0 mmol) and (Z)-3-formyl-N′-hydroxybenzimide (167 mg, 1.02 mmol) according to representative embodiments above. 54% yield (152 mg, 0.54 mmol)

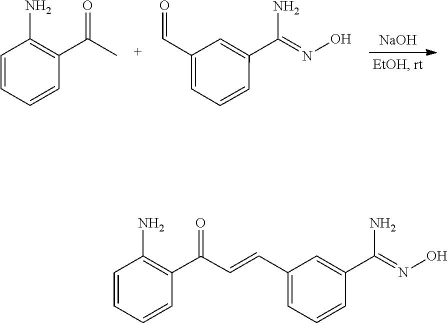

### Manufacturing Example 42. 2-(5-(3-(trifluoromethyl)phenyl)isoxazol-3-yl)aniline (GM-90339)

A solution of guanidine hydrochloride (38 mg, 0.40 mmol) dissolved in N,N-dimethylacetamide (0.5 mL) was treated with sodium ethoxide (28 mg, 0.41 mmol) and stirred for 15 minutes and filtered. The filtrate was added to GM-90283 (58 mg, 0.20 mmol) solution in N, N-dimethylacetamide (0.5 mL), and the resulting mixture was heated to 100° C. for 18 hours while stirring. After completion, the reaction mixture was treated with water (40 mL) and extracted with ethyl acetate (30 mL×3). The extracted organic layer was dried and concentrated on sodium sulfate anhydrous and purified by silica gel column chromatography using a mixture of EA and hexane to obtain GM-90339 at a yield of 54% (34 mg, 0.108 mmol).

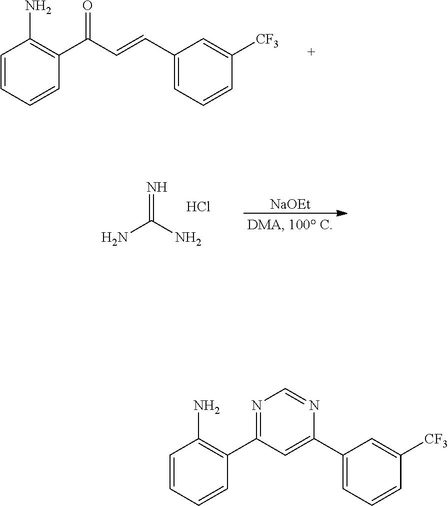

### Manufacturing Example 43. 2-(5-(3-(trifluoromethyl)phenyl)isoxazol-3-yl)aniline (GM-90340)

K₂CO₃ (221 mg, 1.60 mmol) was added to a solution of N-hydroxyl-4-toluenesulfonamide (281 mg, 1.50 mmol) dissolved in 1.4 mL methanol/water (6:1). 0.2 mmol GM-90283 (58 mg) dissolved in 0.6 ml of methanol was then added, and the reaction mixture was stirred at 40° C. for 24 hours and monitored with TLC. An additional K₂CO₃ (111 mg, 0.80 mmol) was then added and the mixture was stirred at 60° C. for 10 hours. After completion of the reaction, the reaction mixture was treated with water (3 mL) and extracted with ethyl acetate (5 m×3). After drying and concentration on sodium sulfate anhydrous, the extracted organic layer was purified by silica gel column chromatography using a mixture of EA and hexane to obtain GM-90340 at a yield of 46% (28 mg, 0.092 mmol).

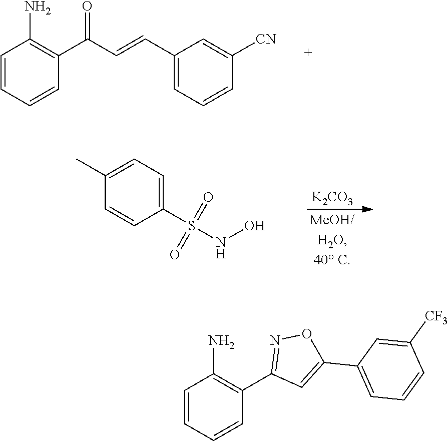

### Manufacturing Example 44. 2E-1-2-Aminophenyl-3-3-nitrophenyl-2-propen-1-one (BIA)

Aq. NaOH solution (NaOH 36 mg/H2O 0.25 mL) in Et0H (10 mL) was kept below 10° C. in an ice bath and 1-(2-aminophenyl) ethane-1-one (100 mg, 0.740 mmol) was added.

The reaction mixture was stirred at 10° C. for 30 minutes. 3-nitrobenzaldehyde (111.8 mg, 0.740 mmol) was then added to the resulting mixture. After stirring at 10° C. for an additional 1 hour, the reaction mixture was stirred at room temperature for an additional 26 hours. EtOH was removed from the vacuum and the residue dissolved in EtOAc. The organic layer was washed with 3N HCl and water. The organic layer was washed with brine and dried and concentrated with sodium sulfate. The crude product was purified by column chromatography to obtain (E)-1-(2-aminophenyl)-3-(3-nitrophenyl)prop-2-n-1-one (164 mg, 82%).

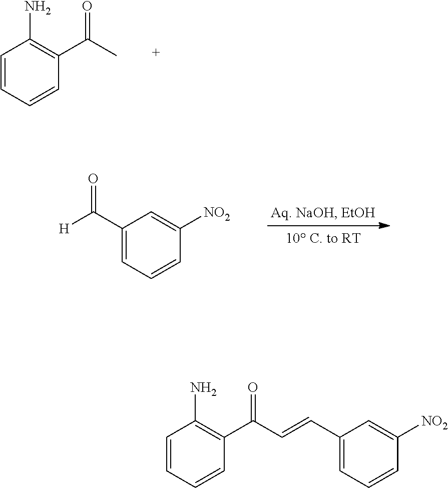

### Example <16> Inhibition of BI-1-Associated Cancer Formation by BI-1 (TMBIM6) Antagonist

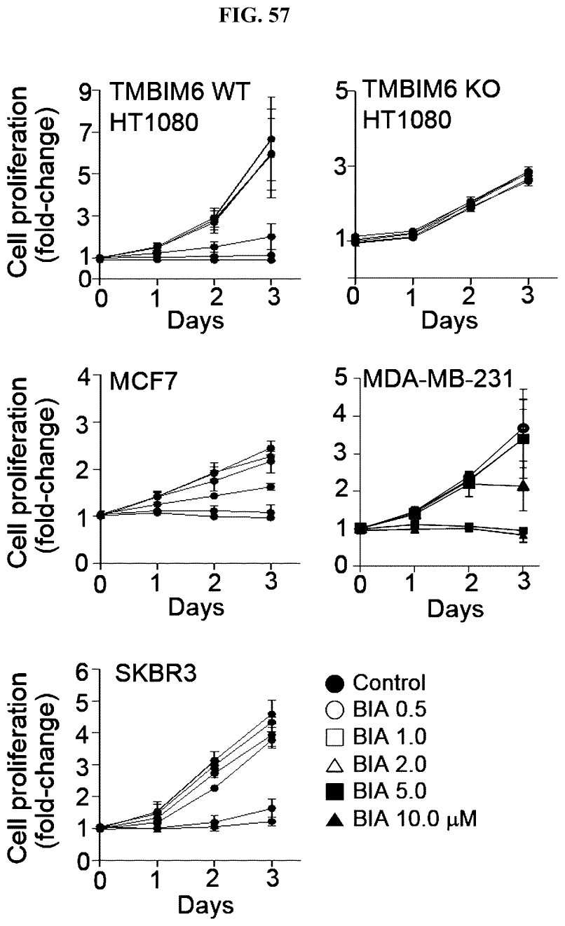

BI-1 (TMBIM6) WT HT1080 cells, BI-1 (TMBIM6) knockout HT1080 , human breast cancer cell lines MCF7, MDA-MB-231 and SKBR3 were treated with BIA 0.5, 1.0, 2.0, 5.0, 10.0 μM and cell proliferation was confirmed. Cells with proliferation of all cell lines by 5 μM BIA treatment It was confirmed that viability was inhibited (see FIG. 57).

The IC₅₀ values obtained by treatment for 3 days are 1.7±0.1 μM for HT1080, 2.6±0.4 μM for MCF cells, 2.6±0.5 μM for MDA-MB-231 cells, and 2.4±0.4 μ for SKBR3 cells. m. In addition, it was found that BI-1 (TMBIM6) knockout cells did not show a decrease in the dose-dependent cell survival of BIA.

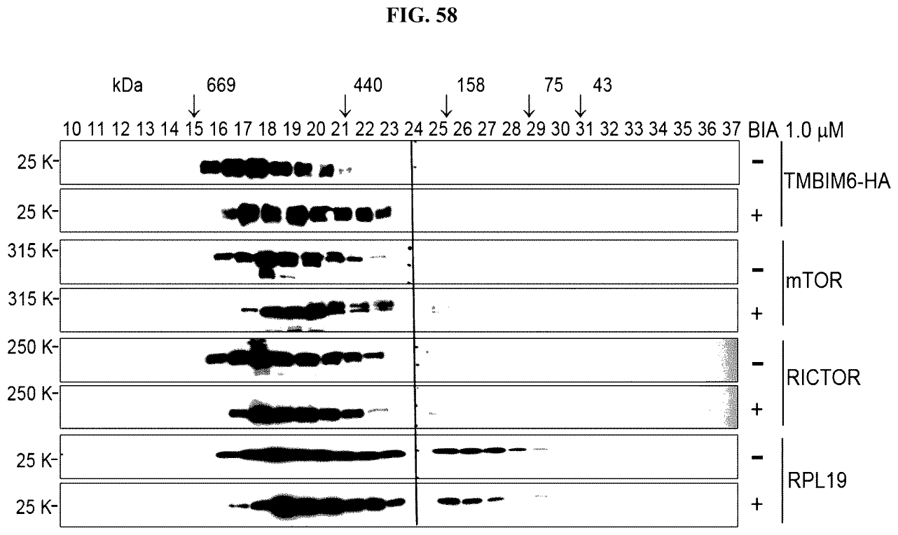

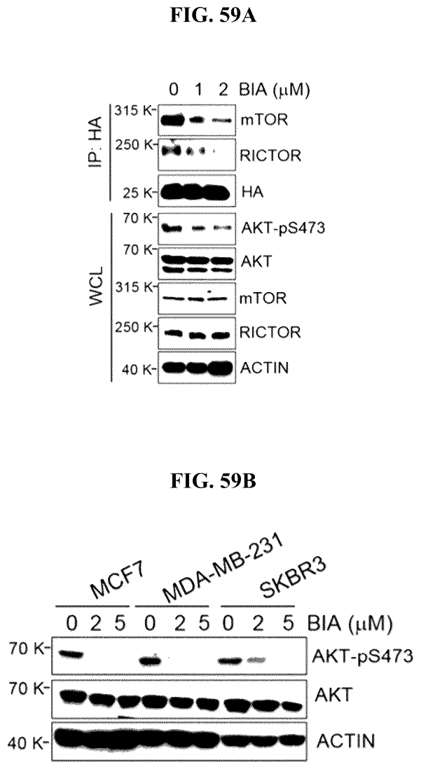

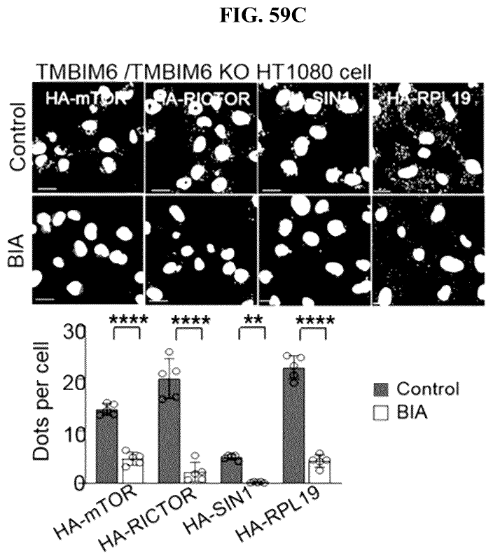

To determine whether BIA reduces the binding between BI-1 and mTORC2, gel filtration assays were performed on BI-1 (TMBIM6) overexpressed HT1080 cells after treatment with BIA for 24 hours. As shown in FIG. 58, the co-elution pattern with mTORC2 components (mTOR and RICTOR) and ribosomes (RPL19) was delayed in BIA-treated HT1080 cells. PLA and immunoprecipitation assays show that endogenous protein interactions between mTORC2 and ribosomes or binding of BI-1 and mTORC2 and ribosomes were inhibited by BIA (see FIGS. 59A-59C), resulting in a complete reduction in phosphorylation of AKT. shows. These results demonstrate that in several cancer cells, BIA induces dissociation of BI-1 from mTORC2 and ribosomes, reducing cell proliferation and resulting in apoptosis.

Cell proliferation in BI-1 (TMBIM6) knockout HT1080 cells was investigated to determine if the antiproliferative effect by BIA at this dose was an off-target effect. The cell proliferation rate and AKT phosphorylation of BI-1 (TMBIM6) knockout HT1080 cells were the same with or without BIA except for high concentrations of “20 and 30 μM” (see FIG. 57). The BIA suggests a targeted effect on BI-1 up to 10 μM.

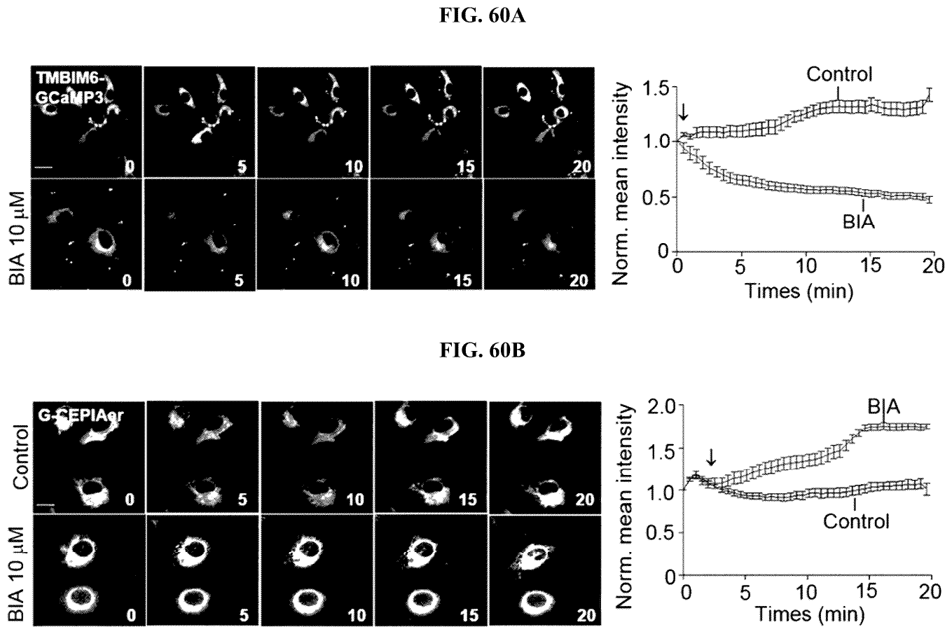

Since BI-1 regulates mTORC2 activation through ER Ca2+ release, the above results confirmed whether BIA inhibits Ca2+ release from BI-1. BI-1-GCaMP3 green fluorescence showed a decreasing pattern in BIA-treated cells (see FIG. 60A, green fluorescence is shown as white). In addition, the ER calcium status was demonstrated using the endoplasmic reticulum (ER) lumen calcium indicator (G-CEPIAer) using a 10 μM BIA. The fluorescence intensity was increased by BIA treatment compared to untreated control cells (see FIG. 60B, green fluorescence is shown in white), which indicates that BIA inhibits the ER release of Ca²⁺ from BI-1. It is suggestive.

### Example <17> Inhibition of Migration and Invasion of Cancer Cells in BIA

Cell migration and invasion are typical in vitro markers for cancer characteristics.

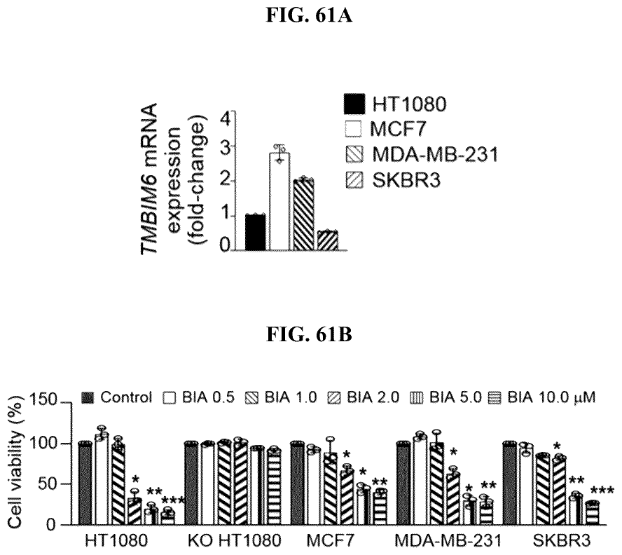

First, we investigated BI-1 (TMBIM6) expression in breast cancer cell lines. MCF7 and MDA-MB-231 cells expressed BI-1 highly, while SKBR3 cells showed low expression compared to HT1080 cells (see FIG. 61A). 5 μM BIA treatment reduces the proliferation and cell viability of all cell lines It was suppressed (see FIG. 61B).

HT1080 cells stably overexpressing BI-1 showed high sensitivity to BIA (see FIG. 62A). PLA analysis shows that endogenous protein interactions between mTORC2 and ribosomes or binding of BI-1 to mTORC2 and ribosomes were inhibited by BIA (see FIG. 62B), resulting in a complete reduction in phosphorylation of AKT.

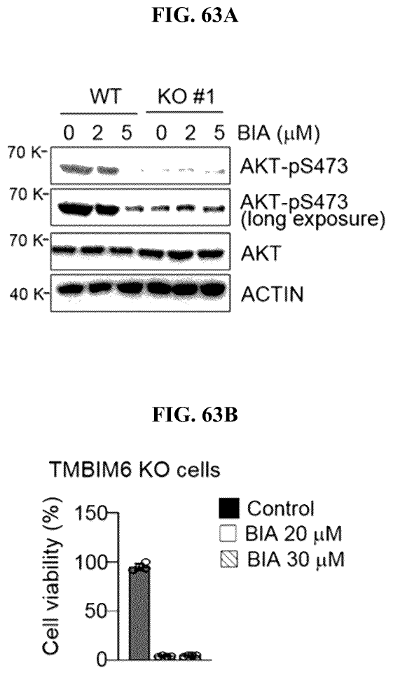

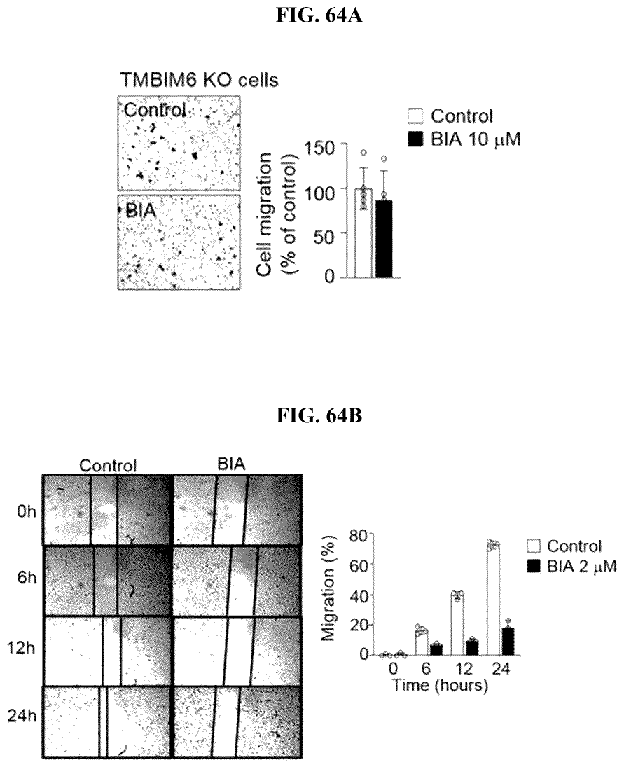

Cell proliferation was investigated in BI-1 (TMBIM6) knockout HT1080 cells to determine if the antiproliferative effect by BIA at the dose was a non-targeted effect. The cell proliferation rate and AKT phosphorylation of BI-1 (TMBIM6) knockout HT1080 cells were the same in the treated and untreated groups of BIA except for high concentrations of “20 and 30 μM” (see FIGS. 63A and 63B). This suggests that BIA has a targeted effect on BI-1 up to 10 μM, and BIA treatment reduced cell migration (see FIGS. 64A and 64B).

### Example <18> Inhibition of AKT Activity and Tumor Progression by BIA Due to the Dissociation of BI-1 From mTORC2

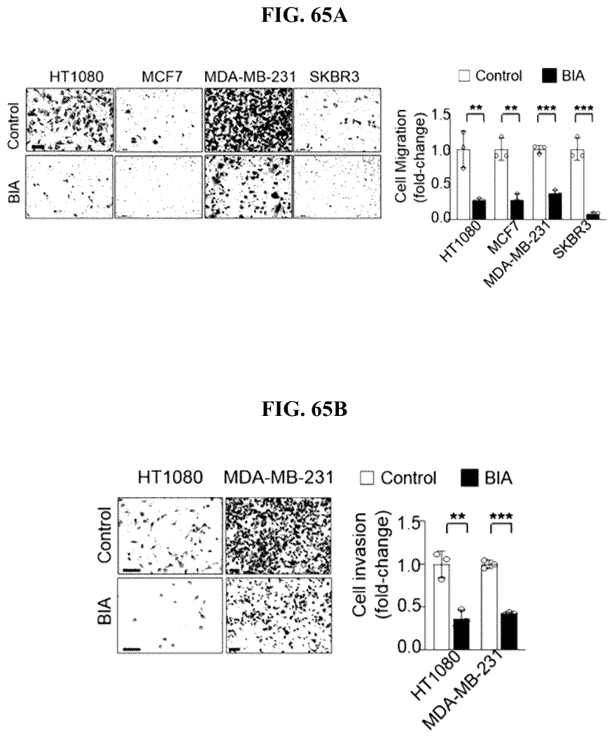

Cell migration and invasion are typical in vitro markers for cancer characteristics. BIA treatment reduced cell migration in HT1080, MCF7, MDA-MB-231 and SKBR3 cells (see FIG. 65A). Cell invasion of MDA-MB-231 and HT1080 cells was also reduced in BIA-treated cells (see FIG. 65B).

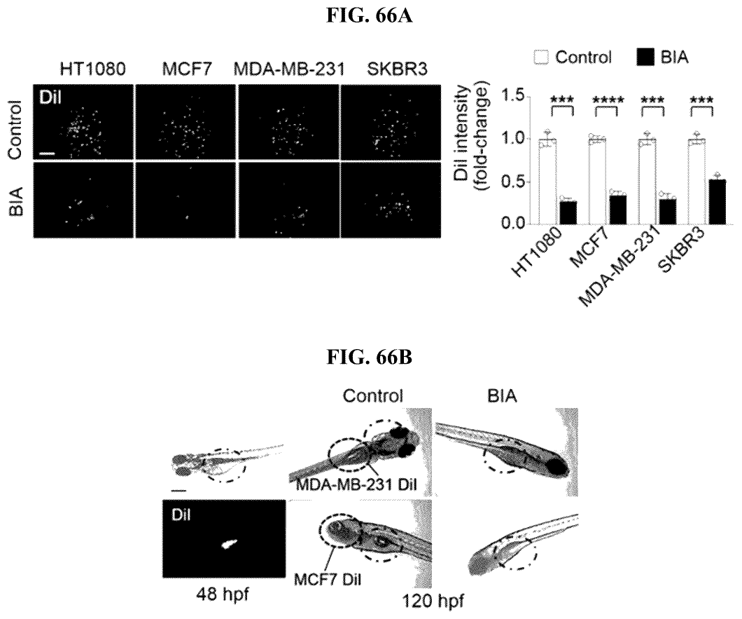

In addition, the number and size of spheroids were formed by three-dimensional cultured cells and did not show multilayer structures damaged by BIA (see FIG. 66A). We established Zebrafish tumor models 48-51 in which human breast cancer cells labeled with DiI dye were injected into the surrounding membrane cavity into embryos 48 hours after fertilization. On the third day after transplantation, the control tumor cells moved away from the primary site, while almost all tumor cells in the BIA-treated group remained at the injection site (see FIG. 66B).

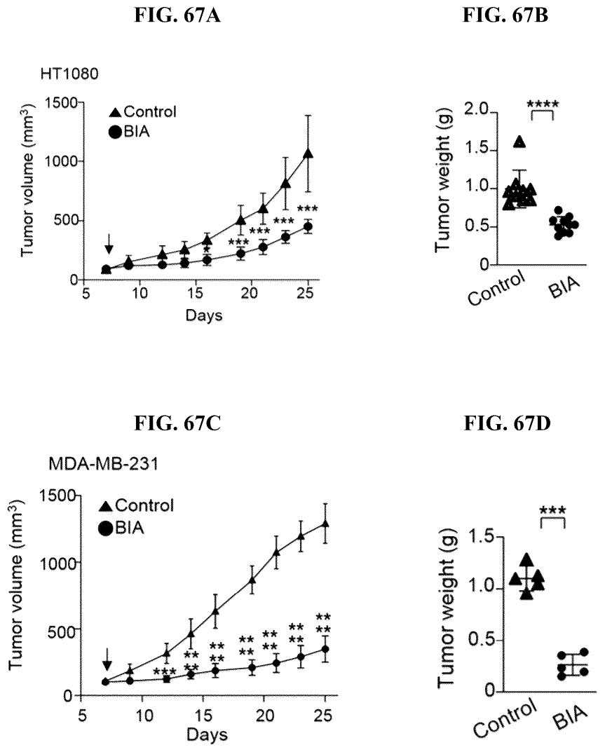

To further determine whether BIA regresses tumor growth in vivo, HT1080 and MDA-MB-231 cells are injected subcutaneously into immunocompromised mice, and 1 mg/kg BIA or vehicle (0.1% DMSO with saline) for 5 days a week Additional injections were made. For 25 days. Xenograft results showed that BIA significantly impaired tumor growth (see FIGS. 67A-67D). These results suggest that inhibition of AKT activity and tumor progression by BIA is due to dissociation of BI-1 from mTORC2.

### Example <19> Reduction of Cancer Cell Survival by BIA

The PIK3CA-AKT-mTOR signaling pathway is frequently activated in human cancers and many small molecule compounds have been developed that target the various pathways of the pathway, but in breast cancer resulting in mTORC1 inhibition, mTOR mutations induce AKT activation through upregulation of receptor tyrosine kinase, resulting in these provokes resistance to inhibitors;

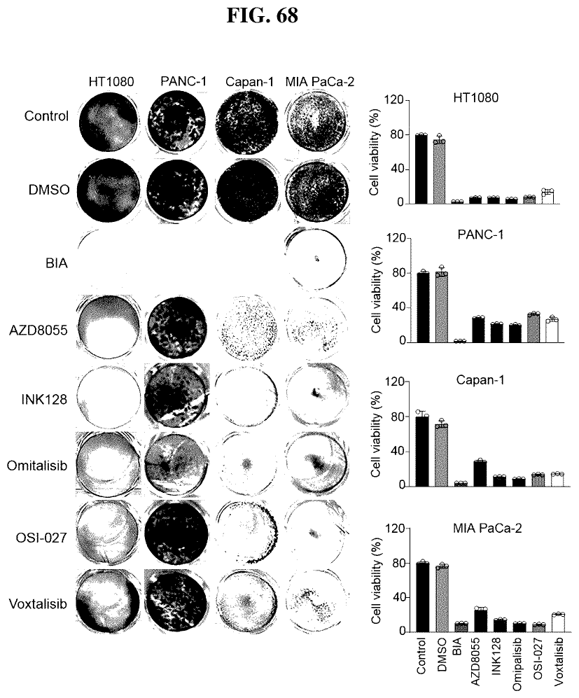

To determine if BIA is effective against PANC-1 pancreatic cancer cells resistant to HT1080, mTOR inhibitors, and other pancreatic cancer cells, including Capan-1 and MIA PaCa-2 cells, we compared it with mTOR inhibitor anticancer drugs such as AZD8055, INK128, Omitalisib, and OSI . The BIA treatment group significantly reduced cell viability compared to OSI-027 and Voxtalisib and other mTOR inhibitors. In particular, BIA almost removed live cells from PANC-1 cells (see FIG. 68).

From the description of the present disclosure, it can be seen that BIA has a better effect than well-known anticancer agents.

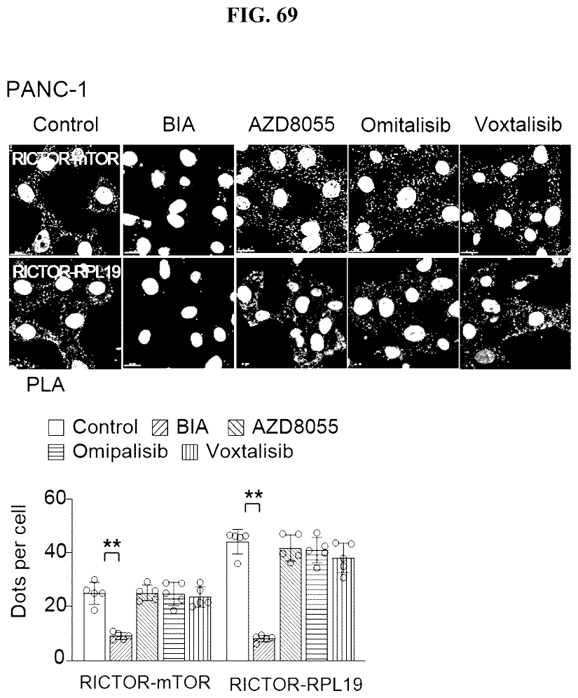

In PLA analysis, BIA reduced the association between RICTOR and mTOR or between RICTOR and RPL19, but mTOR inhibitors did not affect any association in PANC-1 cells (see FIG. 69), which indicates that BIA controls cancer cells It suggests that it has potential as an effective anticancer agent.

### Example <20> Cell Fertility Experiment on BIA and Its Analogs

The compound prepared in Example 15 was treated with HT1080 fibrosarcoma cell line at 5 μM and 10 μM and cell viability was measured. In addition, the compound prepared in Example 15 on the DU145 prostate cancer cell line was treated with 10 μM, 20 μM, and 30 μM and cell viability was measured (see Table 2, Table 3).

TABLE 2
Cell viability for BIA and analogues of HT1080 cell line

| | 10 uM | | 5 uM | |
|---|---|---|---|---|
| | AVERAGE | STDEV | AVERAGE | STDEV |
| Control | 100.00 | 0.18 | 99.87 | 0.03 |
| DMSO | 101.13 | 0.20 | 101.23 | 0.16 |
| BEER | 7.21 | 0.46 | 19.93 | 0.00 |
| GM-90128 | 57.83 | 3.45 | 101.56 | 0.37 |
| GM-90129 | 24.34 | 1.23 | 97.92 | 0.71 |
| GM-90130 | 31.69 | 2.12 | 99.48 | 0.49 |
| GM-90131 | 37.42 | 1.34 | 100.89 | 0.17 |
| GM-90132 | 30.94 | 1.08 | 100.10 | 0.07 |
| GM-90133 | 54.86 | 1.55 | 100.86 | 0.08 |
| GM-90134 | 96.62 | 0.05 | 99.10 | 0.09 |
| GM-90135 | 96.82 | 2.21 | 99.92 | 0.01 |
| GM-90222 | 92.00 | 4.44 | 108.75 | 1.78 |
| GM-90223 | 85.95 | 6.00 | 93.80 | 7.67 |
| GM-90224 | 99.67 | 6.44 | 108.14 | 8.78 |
| GM-90225 | 43.46 | 7.13 | 70.46 | 8.40 |
| GM-90226 | 15.38 | 1.85 | 83.91 | 2.53 |
| GM-90227 | 96.30 | 4.84 | 108.46 | 0.57 |
| GM-90228 | 22.77 | 2.10 | 30.45 | 0.91 |
| GM-90229 | 15.73 | 0.47 | 64.43 | 0.16 |
| GM-90230 | 98.80 | 1.77 | 108.35 | 1.87 |
| GM-90243 | 39.20 | 1.34 | 66.84 | 3.04 |
| GM-90254 | 8.59 | 0.11 | 14.69 | 0.33 |
| GM-90255 | 33.43 | 3.29 | 88.06 | 0.94 |
| GM-90256 | 46.30 | 6.71 | 82.40 | 0.71 |
| GM-90281 | 11.13 | 0.10 | 11.36 | 0.04 |
| GM-90282 | 7.14 | 0.10 | 12.23 | 0.08 |
| GM-90283 | 11.94 | 0.25 | 18.08 | 0.12 |
| GM-90284 | 18.81 | 0.23 | 86.44 | 0.85 |
| GM-90285 | 8.35 | 0.12 | 71.54 | 1.44 |
| GM-90295 | 82.42 | 1.19 | 103.39 | 0.64 |
| GM-90296 | 91.62 | 1.38 | 107.05 | 1.86 |
| GM-90297 | 18.20 | 1.72 | 93.28 | 1.82 |
| GM-90298 | 18.96 | 1.87 | 21.18 | 2.06 |
| GM-90299 | 20.10 | 1.33 | 93.15 | 3.17 |
| GM-90300 | 23.40 | 1.65 | 99.48 | 5.22 |
| GM-90315 | 11.19 | 0.38 | 41.10 | 5.94 |
| GM-90316 | 67.82 | 7.15 | 100.61 | 9.08 |
| GM-90317 | 14.40 | 2.81 | 46.75 | 9.53 |
| GM-90318 | 11.83 | 1.40 | 21.37 | 1.55 |
| GM-90319 | 9.94 | 1.26 | 46.62 | 0.78 |
| GM-90320 | 62.73 | 7.03 | 94.54 | 6.05 |
| GM-90321 | 21.25 | 2.94 | 61.16 | 0.46 |
| GM-90337 | 21.42 | 2.15 | 83.67 | 2.12 |
| GM-90338 | 10.49 | 1.66 | 44.67 | 1.75 |
| GM-90339 | 97.30 | 0.98 | 99.61 | 4.18 |
| GM-90340 | 80.21 | 1.57 | 88.71 | 0.06 |

TABLE 3
Cell viability for BIA and analogues of DU145 cell line

| | 10 uM | | 20 uM | | 50 rpm | |
|---|---|---|---|---|---|---|
| | AVERAGE | STDEV | AVERAGE | STDEV | AVERAGE | STDEV |
| Control | 100.00 | 0.18 | 100.00 | 0.06 | 100.00 | 0.40 |
| DMSO | 99.96 | 0.11 | 99.66 | 0.14 | 99.47 | 0.33 |
| GM-90128 | 80.96 | 0.69 | 72.24 | 1.45 | 62.19 | 1.39 |
| GM-90129 | 91.39 | 0.43 | 90.18 | 0.03 | 61.39 | 2.05 |
| GM-90130 | 78.72 | 2.36 | 79.36 | 0.40 | 48.29 | 3.23 |
| GM-90131 | 81.53 | 1.57 | 74.04 | 10.12 | 33.21 | 2.96 |
| GM-90132 | 81.45 | 1.75 | 71.19 | 2.20 | 19.89 | 3.63 |
| GM-90133 | 50.54 | 9.99 | 35.23 | 1.81 | 26.75 | 2.38 |
| GM-90134 | 31.47 | 1.35 | 25.37 | 2.20 | 22.80 | 0.07 |
| GM-90135 | 88.51 | 0.36 | 87.33 | 1.60 | 64.55 | 0.16 |
| GM-90222 | 40.68 | 0.48 | 31.27 | 0.11 | 28.95 | 0.35 |
| GM-90223 | 26.48 | 1.38 | 26.34 | 0.63 | 21.14 | 1.49 |
| GM-90224 | 90.46 | 0.58 | 90.00 | 0.43 | 75.80 | 1.44 |
| GM-90225 | 87.41 | 0.23 | 79.44 | 3.49 | 71.18 | 4.27 |
| GM-90226 | 68.10 | 0.04 | 46.76 | 4.22 | 11.84 | 0.57 |
| GM-90227 | 43.32 | 2.55 | 36.08 | 1.25 | 28.20 | 5.05 |
| GM-90228 | 54.08 | 3.26 | 37.77 | 1.74 | 15.05 | 0.38 |
| GM-90229 | 53.11 | 1.63 | 40.51 | 9.87 | 21.14 | 2.75 |
| GM-90230 | 66.34 | 12.08 | 29.14 | 0.86 | 29.30 | 1.20 |
| GM-90243 | 31.80 | 1.63 | 28.19 | 0.20 | 14.29 | 0.40 |
| GM-90254 | 61.08 | 2.34 | 31.52 | 0.51 | 8.51 | 0.16 |
| GM-90255 | 88.67 | 0.39 | 70.18 | 1.43 | 66.43 | 2.46 |
| GM-90256 | 76.31 | 0.95 | 60.23 | 2.12 | 54.94 | 1.85 |
| GM-90281 | 77.43 | 1.18 | 76.39 | 4.28 | 67.05 | 0.79 |
| GM-90282 | 72.93 | 0.64 | 53.36 | 6.88 | 9.57 | 0.17 |
| GM-90283 | 59.94 | 1.89 | 33.06 | 0.84 | 22.86 | 1.98 |
| GM-90284 | 88.41 | 2.57 | 81.00 | 0.80 | 79.05 | 0.23 |
| GM-90285 | 83.47 | 0.79 | 82.84 | 1.08 | 78.76 | 1.05 |
| GM-90295 | 91.72 | 0.75 | 86.74 | 0.26 | 84.68 | 0.65 |
| GM-90296 | 91.04 | 0.58 | 82.30 | 0.74 | 76.94 | 0.24 |
| GM-90297 | 85.70 | 2.37 | 87.18 | 2.51 | 83.04 | 2.50 |
| GM-90298 | 68.94 | 0.95 | 62.31 | 2.97 | 48.73 | 0.46 |
| GM-90299 | 82.16 | 0.43 | 59.03 | 0.41 | 17.21 | 0.30 |
| GM-90300 | 62.60 | 0.43 | 58.51 | 1.47 | 54.46 | 4.43 |
| GM-90315 | 98.04 | 0.01 | 97.94 | 0.03 | 88.88 | 0.03 |
| GM-90316 | 87.40 | 1.77 | 60.00 | 3.28 | 38.38 | 0.39 |
| GM-90317 | 32.39 | 0.70 | 28.11 | 0.73 | 11.94 | 0.16 |
| GM-90318 | 44.64 | 1.44 | 43.09 | 1.11 | 13.39 | 0.17 |
| GM-90319 | 109.67 | 3.09 | 88.14 | 0.25 | 85.75 | 0.63 |
| GM-90320 | 89.08 | 0.98 | 89.08 | 0.89 | 82.37 | 2.14 |
| GM-90321 | 83.59 | 1.06 | 81.80 | 1.11 | 84.79 | 0.88 |
| GM-90337 | 61.70 | 0.32 | 39.06 | 1.14 | 41.40 | 0.78 |
| GM-90338 | 42.78 | 3.38 | 31.89 | 3.23 | 11.13 | 0.12 |
| GM-90339 | 95.29 | 1.84 | 87.82 | 0.87 | 74.80 | 0.43 |
| GM-90340 | 92.89 | 0.66 | 89.29 | 0.66 | 76.07 | 2.20 |

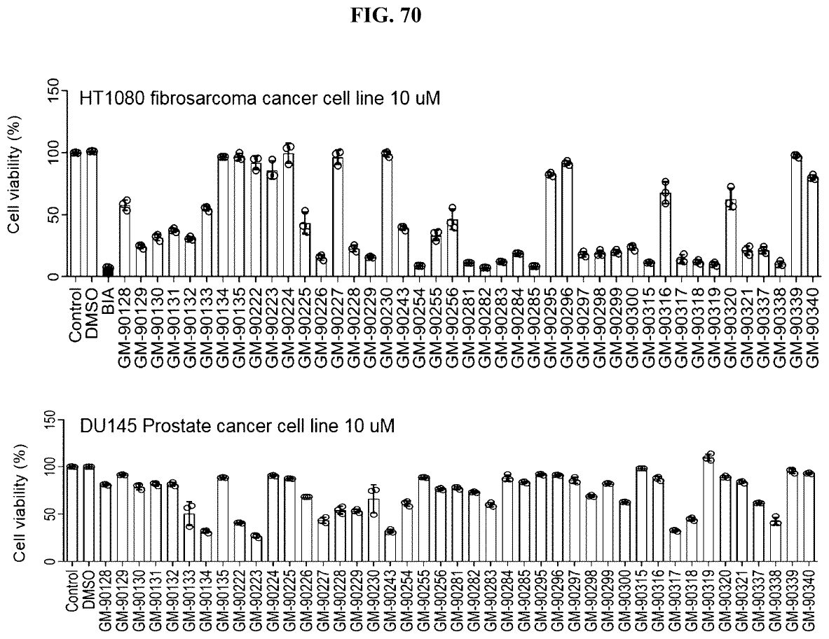

FIG. 70 shows the results of treating 10 uM of the compound prepared in Example 15 with fibrosarcoma cells and prostate cancer cell lines and confirming cell viability.

### Example <21> Inhibition of AKT Phosphorylation by BIA and its Analogues

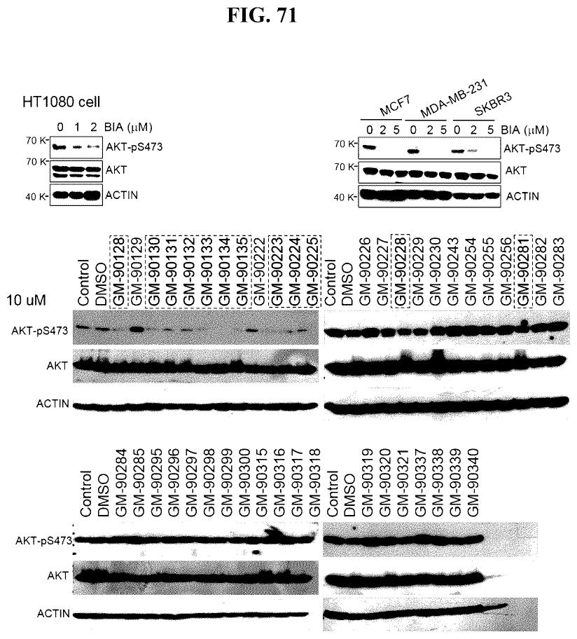

When BIA was treated at a concentration of 1 or 2 μM, it was confirmed that it inhibited AKT serine phosphorylation S473 in HT1080 cells, and MDA-MB-231 cells and SKBR3 cells were equally inhibited to inhibit the phosphorylation of AKT (see FIG. 71).

Breast cancer cells including HT1080 cells, MCF cells, MDA-MB-231 cells, and SKBR3 cells were cultured and treated with 10 μM of BIA analogues.

In the compounds marked with the following dotted box, including GM-90128, AKT serine phosphorylation was inhibited at a concentration of 10 μM. In other compounds, higher concentrations than 10 μM were required for complete inhibition of AKT, but overall inhibition tended (see FIG. 71).

### Example <22> Inhibition of Migration and Invasion of Cancer Cells by BIA and its Analogues

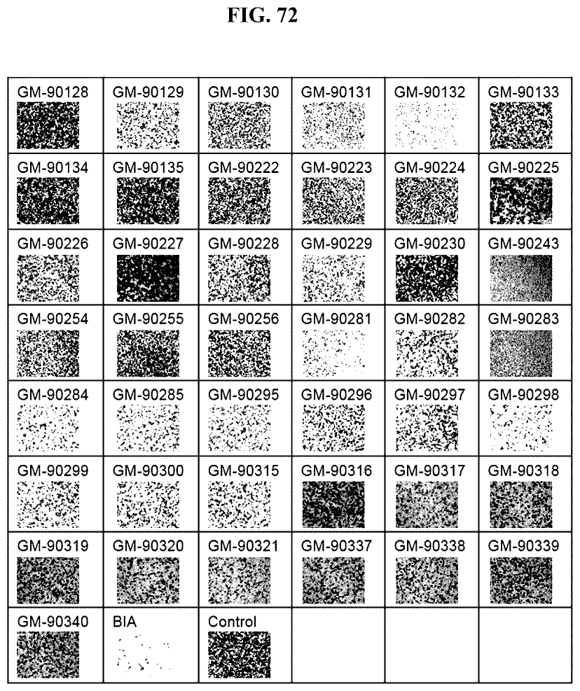

BIA and its analogues HT1080 measured cell migration characteristics of cancer cells after treatment on fibrosarcoma cells. BIA strongly inhibited the migration of cancer cells, and other compounds also showed significant inhibition (see FIG. 72).

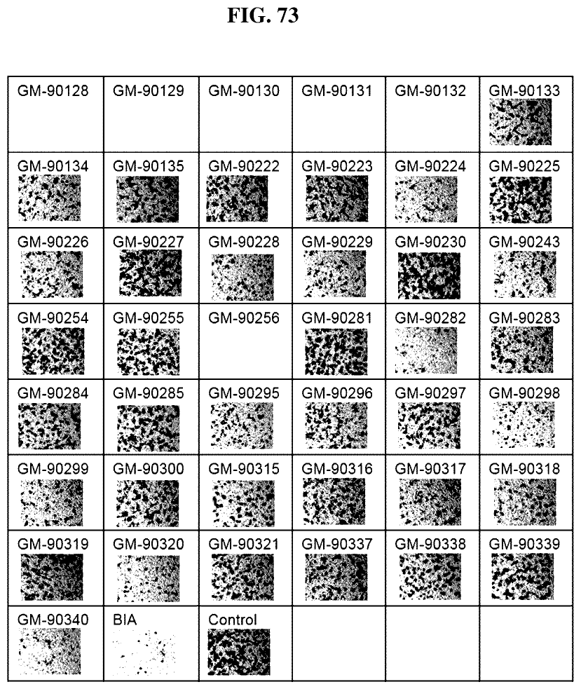

BIA and its analogues HT1080 measured cell invasion characteristics of cancer cells after treatment on fibrosarcoma cells. BIA showed a very pronounced cell invasion inhibition phenomenon among the compounds measured, and other compounds also showed significant inhibition (see FIG. 73).

### Example <23> Free Inhibition of Ca2+ in Endoplasmic Reticulum by BIA

The most basic property of BIA is that it “prevents calcium-free (leakage) in the endoplasmic reticulum (ER) of the BI-1 (TMBIM6) protein.” The key mechanism is that mTORC2 and ribosomes are recruited through this. To confirm this, BTMBIM6-GCaMP3 plasmid was first made and transfection HT1080 cells to create stable cells. These transfected cells can only be used to detect calcium coming out through BI-1.

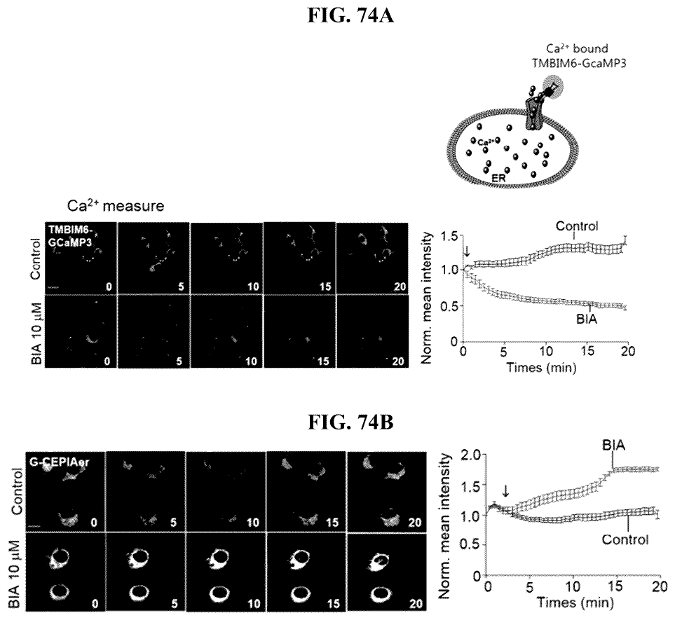

BIA 10 μM was treated with transfected cells, the fluorescence intensity of GCaMP3 was observed over time, and it was confirmed that the fluorescence intensity decreased over time (see FIG. 74A).

On the other hand, when BIA is treated, calcium inside ER increases because nine in ER inhibits calcium. This was expressed G-CEPIAer, which measures the calcium concentration inside ER, and metamorphed HT1080 cells using CEPIAer plasmid to create stable cells. This transfected cell can be used to detect only free calcium from ER via BI-1 (TMBIM6) based on the calcium channel-like nature of BI-1 (TMBIM6). By confirming the increase in fluorescence, it was possible to confirm the specificity of the BIA (see FIG. 74B).

BIA has been proven to inhibit calcium free from the endoplasmic reticulum, precisely the basic properties of BI-1.

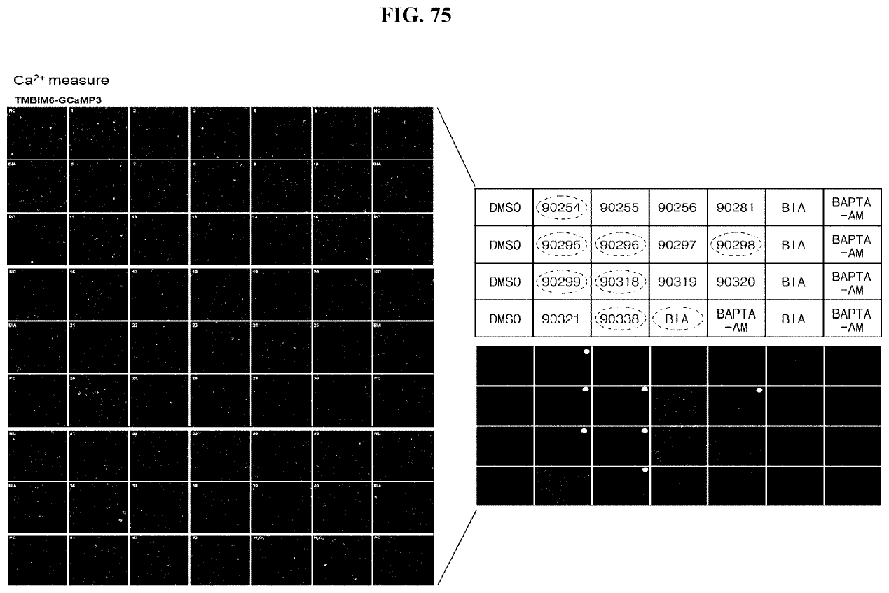

When the analogues including BIA were treated with cells under control (DMSO treatment conditions) conditions in which the fluorescence of TMBIM6-GCaMP3 was expressed, the calcium image coming out through BI-1 was confirmed by confirming the change in fluorescence (see FIG. 75).

Most suppressed calcium glass to some extent. In particular, the following compounds showed a stronger inhibitory effect.
(E)-1-(2-amino-4,5-dimethoxyphenyl)-3-(3-nitrophenyl)prop-2-en-1-one (GM-90254),
(E)-1-(2-aminophenyl)-3-(3-ethoxyphenyl)prop-2-en-1-one (GM-90295),
(E)-1-(2-amino-5-fluorophenyl)-3-(3-nitrophenyl) prop-2-en-1-one (GM-90298),
tert-butyl (E)-(3-(3-(2-aminophenyl)-3-oxoprop-1-en-1-yl)phenyl)carbamate (GM-90299),
(E)-3-(3-(2-aminophenyl)-3-oxoprop-1-en-1-yl)benzoic acid (GM-90296),
(E)-1-(2-aminophenyl)-3-(3-fluorophenyl)prop-2-en-1-one (GM-90318),
-(Z)-3-((E)-3-(2-aminophenyl)-3-oxoprop-1-en-1-yl)-N′-hydroxybenzimid amide (GM-90338)

### Example <24> Inhibition of Inflammatory Cell Growth in Asthma Model by Knockout of BI-1

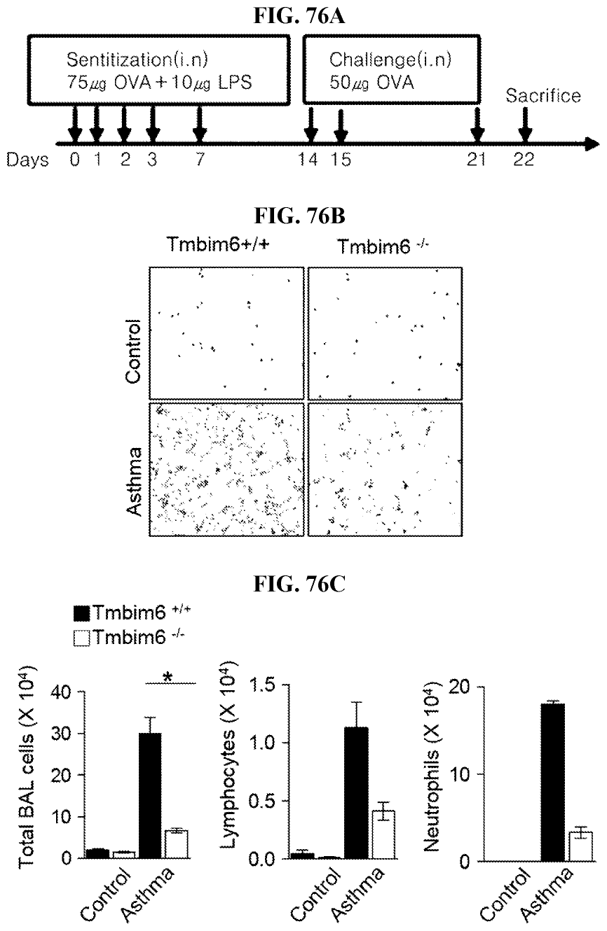

As shown in FIG. 76A, OVA and LPS were treated with BI-1 (TMBIM6)WT WT(+/+) mice and knockout KO mice (−/−) to induce asthma.

On day 22 (3 hours after the last airway challenge with OVA), 1 mg/kg of IC87114 (PI3K inhibitor), 0.01 mg/kg or 0.1 mg/kg of BIA or 0.9% NaC1 (0.05% dimethyl sulfoxide) diluted in 30 μl It was administered by volume by the endotracheal non-surgical method Inflammatory cells of bronchoalveolar lavage fluid (BAL) were very increased in the erotic group of WT mice and were inhibited in BI-1 (TMBIM6) knockout mice (see FIG. 76B).

BAL cell counts and lymphocytes and neutrophil counts were also increased in the asthma-inducing group of WT mice and were inhibited in BI-1 (TMBIM6) knockout mice (see FIG. 76C).

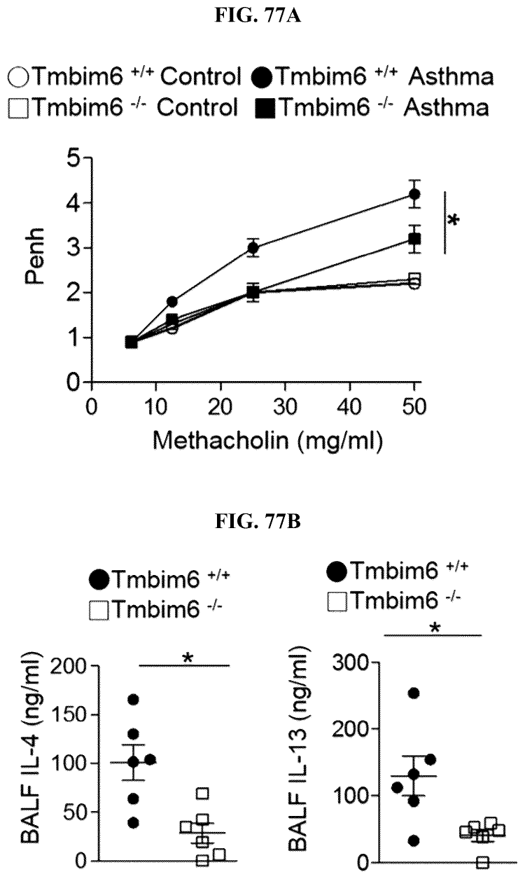

Measurement of airway hypersensitivity with the Methacholine test showed increased sensitivity in WT asthma-inducing mice (+/+ group) (see FIG. 77A).

Concentrations of interleukin (IL)-4 and IL-13 in the entire BALP were measured using individual enzyme-linked immunosorbent assay kits according to the manufacturer's instructions (BD Biosciences, San Jose, CA, USA). IL-4 and IL-13 cytokines were significantly increased in WT asthma-inducing mice and the increase was inhibited in knockout asthma-inducing mice (see FIG. 77B).

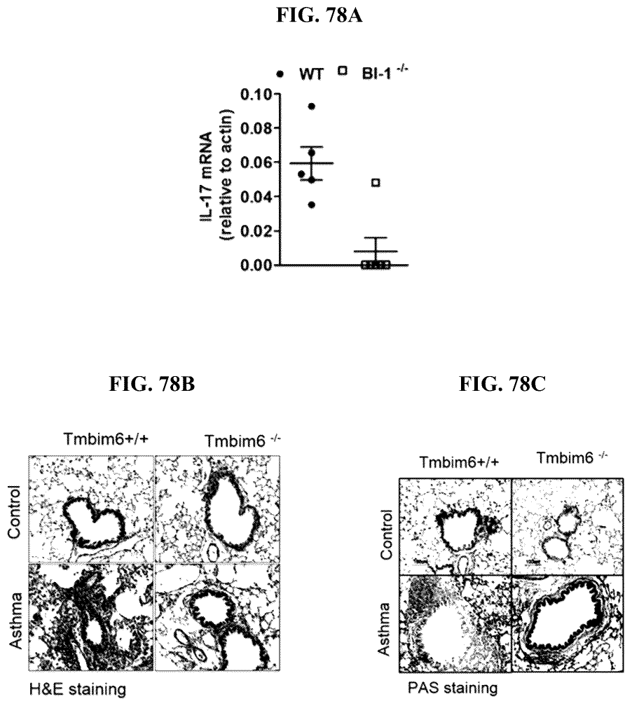

There was also a difference in expression levels of IL-17 mRNA which showed that it was inhibited in knockout asthma-inducing mice (see FIG. 78A).

Observations performed by hematoxylin-eosin staining showed that the airway was restored to normal under knockout conditions (see FIG. 78B). As a result of observation by PAS staining, it was observed that fibrosis of the lungs occurred when asthma was induced under WT conditions, and fibrosis was inhibited under KO conditions (see FIG. 78C).

### Example <25> Inhibition of Inflammation Due to Activation of AKT by BIA

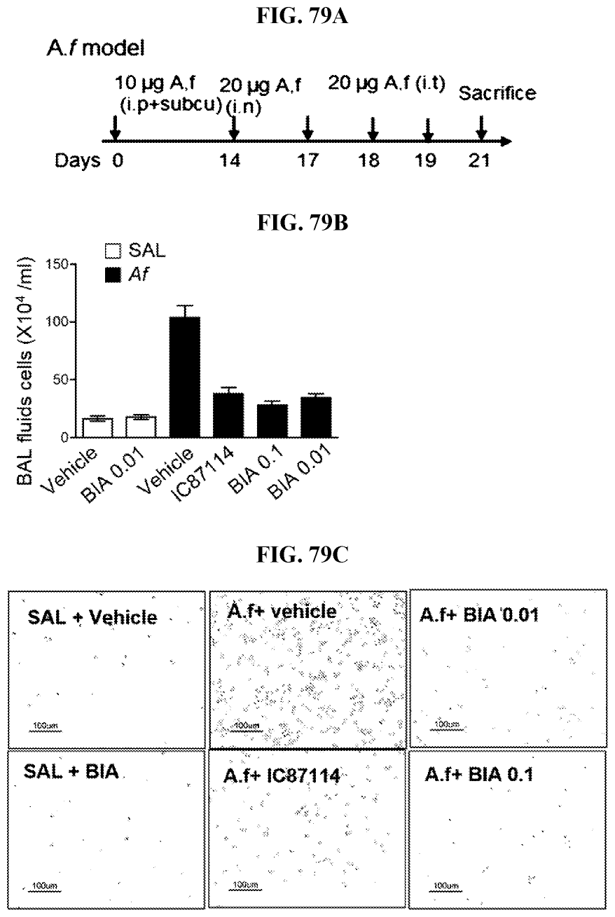

As shown in FIG. 79A, a female C57BL/6 mouse aged 7-8 weeks is a Korean Seongnam Orient Bio Inc. The mice were housed at 22±1° C. with a 12-hour contrast period and freely fed regular feed and water under standard conditions (no specific pathogens) with air filtration. A. fumigatus-induced allergic pulmonary inflammation To establish the model, mice were treated with 10 μg of A. fumigatus crude antigen extract (Greer Laboratories, Cat # XPM3D3A4, Lenoir, NC, USA) with fungal substance inactivated. 0.2 ml of incomplete Freund's adjuvant (Sigma-Aldrich, Cat # F5506-6X) in regular saline It was dissolved and mixed, and half of the preparation was deposited in the peritoneal cavity and the rest was delivered subcutaneously. After 2 weeks, mice received 20 μg of A. fumigatus antigen dissolved in normal saline through the intranasal route, after an intranasal attack After 4 days, 20 μg of A. Fumigatus antigen dissolved in normal saline was treated via the endotracheal route. Control mice were administered only saline at the same time point in time through the same route and treated with the same number of conidia. Bronchoalveolar lavage (BAL) was performed 48 hours after the last challenge with A. fumigatus.

0.1 mg/kg and 0.01 mg/kg, 1 mg/kg IC87114 (PI 3K inhibitor) of BIA were injected intratracheal. Even in control conditions, BIA 0.01 mg/kg was treated. In the asthma-inducing group, all BAL inflammatory cells were inhibited in the BIA, 1 mg/kg IC87114 treatment group (see FIGS. 79B and 79C).

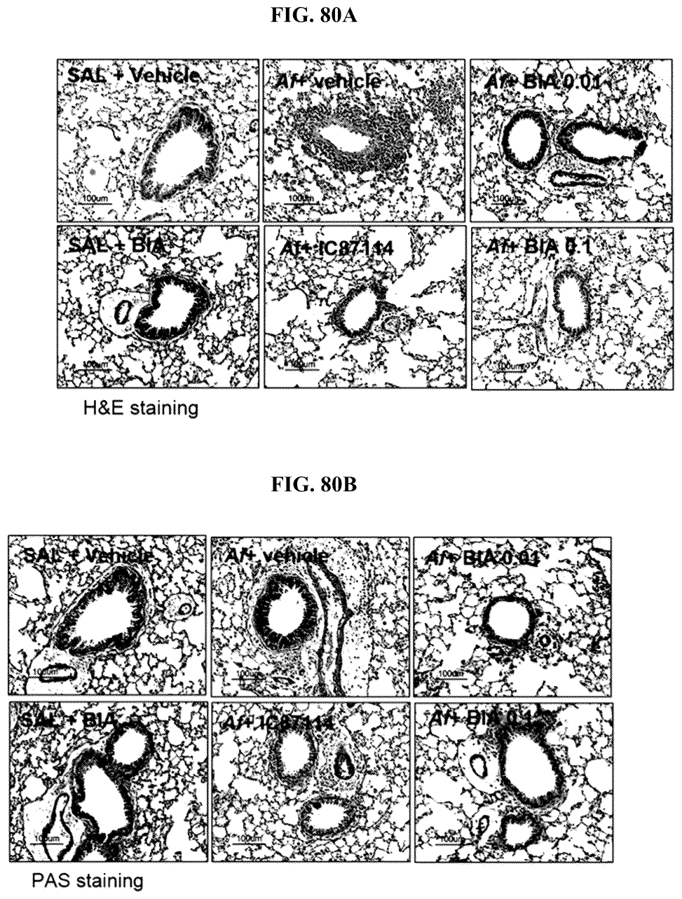

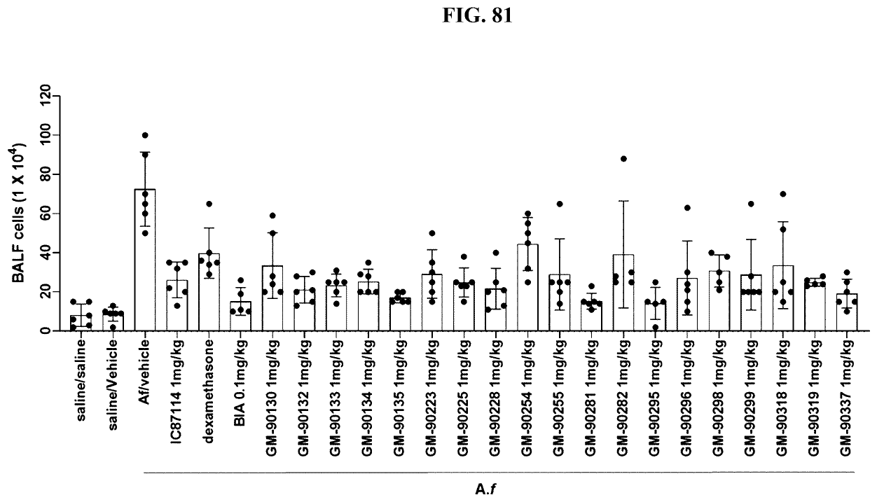

Observation by performing hematoxillin-eosin staining showed that the airway was restored to normal in the drug treatment group (see FIG. 80A). As a result of observation performed by PAS staining, it was confirmed that fibrosis and the like were weakened in the drug treatment group (see FIG. 80B).

These results show that the treatment of BIA to inhibit the activity of BI-1, which has been demonstrated by mTORC2-AKT activation under conditions of increased inflammation by activating AKT (PI3K substep phosphorylase), and the same as treatment with PI3K inhibitor IC87114. This shows the same mechanism of BIA mentioned earlier.

In an aspergillus-infected asthmatic mouse model, IC87114 (PI3K inhibitor) 1 mg/kg, dexamethasone (1 mg/kg body weight/day, Sigma-Aldrich, St Louis, Missouri, USA), BIA 0.1 mg/kg or BIA analogues of the following compounds were treated and quantified by counting the total number of cells in BAL fluid (see FIG. 81).
(E)-1-(2-aminophenyl)-3-phenylprop-2-en-1-one (GM-90130),
(E)-1-(2-aminophenyl)-3-(3-bromophenyl)prop-2-en-1-one (GM-90132),
(E)-1-(2-aminophenyl)-3-(4-nitrophenyl)prop-2-en-1-one (GM-90133),
(E)-1-(2-methoxyphenyl)-3-(3-nitrophenyl)prop-2-en-1-one (GM-90134),
(E)-1-(2-hydroxyphenyl)-3-(3-nitrophenyl)prop-2-en-1-one (GM-90135),
(E)-1-(2-aminophenyl)-3-(2,4-difluorophenyl)prop-2-en-1-one (GM-90223),
(E)-1-(2-aminophenyl)-3-(naphthalen-2-yl)prop -2-en-1-one (GM-90225),
(E)-1-(2-aminophenyl)-3-(pyridin-4-yl)prop-2-en-1-one (GM-90228),
(E)-1-(2-amino-4,5-dimethoxyphenyl)-3-(3-bromophenyl)prop-2-en-1-one (GM-90255),
(E)-1-(2-amino-4,5-dimethoxyphenyl)-3-(3-nitrophenyl)prop-2-en-1-one (GM-90254),
(E)-1-(2-aminophenyl)-3-(3-bromo-5-hydroxyphenyl)prop-2-en-1-one (GM-90281),
(E)-3-(3-(2-aminophenyl)-3-oxoprop-1-and-1-yl)benzonitrile (GM-90282),
(E)-1-(2-aminophenyl)-3-(3-ethoxyphenyl)prop-2-en-1-one (GM-90295),
(E)-3-(3-(2-aminophenyl)-3-oxoprop-1-en-1-yl)benzoic acid (GM-90296),
(E)-1-(2-amino-5-fluorophenyl)-3-(3-nitrophenyl)prop-2-en-1-one (GM-90298),
tert-butyl (E)-(3-(3-(2-aminophenyl)-3-oxoprop-1-en-1-yl)phenyl)carbamate (GM-90299),
(E)-1-(2-aminophenyl)-3-(3-fluorophenyl)prop-2-en-1-one (GM-90318),
(E)-3-(3-(2-amino-4,5-dimethoxyphenyl)-3-oxoprop-1-en-1-yl)benzonitrile (GM-90319)

Compared with the vehicle treatment on mice infected with aspergillus, the increase in cell count of BALP was inhibited in all of the above drugs when treated with the drugs.

PI3K is a super factor of AKT, and PI3K inhibitors inhibit AKT, suggesting the same implications as inhibiting AKT activation, which is the result of signaling of BI-1. BIA was able to prevent asthma from worsening by inhibiting AKT activation.

<Example 26> Anti-SARS-CoV2 Viral Effects by BIA and Its Analogues

Vero E6 cells, monkey kidney cell masters, were dispensed at 1×10⁴ cells/well per well in 96 well plates and incubated at 37° C., 5% CO₂ incubators for 24 hours. SARS-CoV2 to infect Vero E6 cells was S ARS-CoV 43326 distributed from the National Pathogen Resource Bank, and DMEM (2% FBS, 1% antibiotic-antimycotic) medium was dispensed at 100 μL/well to infect 0.1 MOI. For 1 hour The virus was removed after 1 hour of infection in a 37° C., 5% CO₂ incubator.

Next, samples prepared so that BIA or analogue compounds were finally 500 nM were dispensed 100 μL/well, including the culture medium, and then treated in a 37° C., 5% CO₂ incubator for 24 hours. In the same way, the group treated with 2 μM, 5 μM, or 10 μM of Remdesivir (Rem) was a positive control.

Check the condition of the cells, add 100 μL/well of 0.5 mg/ml MTT solution, react in a 37° C., 5% CO₂ incubator for 4 hours, wash out the media, and DMSO to the cells by 100 μL/well After sufficient dissolution, absorbance was measured at 540 nm with a plate reader to evaluate cell viability.

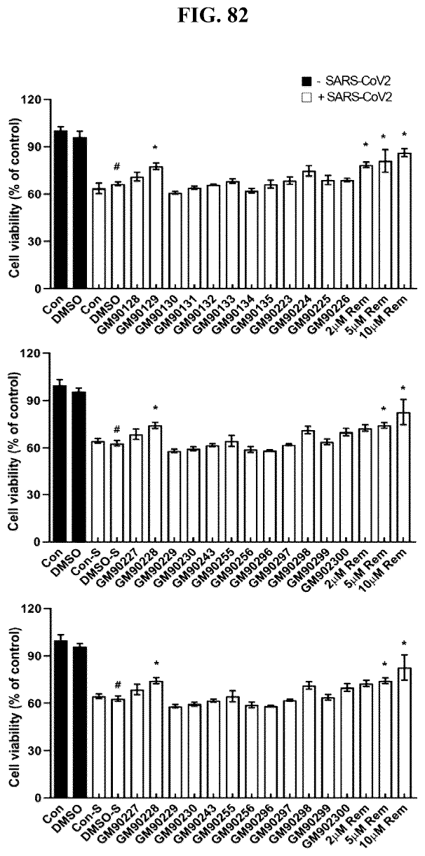

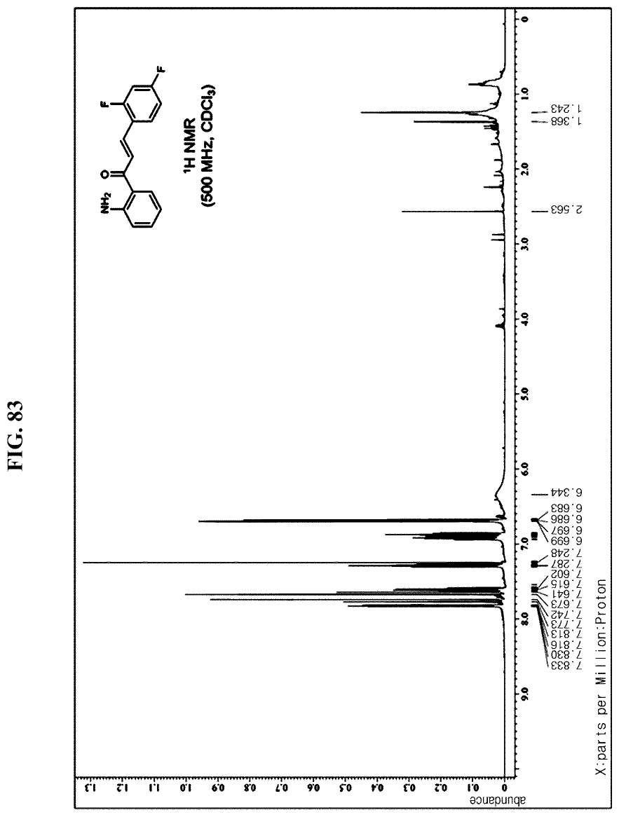

To confirm the possibility of viral prevention and therapeutic effects of BIA and its analogue compounds, 500 nM treatment of BIA analogue at the same time as viral infection, and then MTT assay whether the viability of cells against viral infection is improved after 24 hours. The results confirmed are shown in the graph of FIG. 82.

From the graph of FIG. 82, the normal group (the group that did not treat the SARS-CoV2 virus; Con, DMSO) and the control group showed that the cell viability level of the control group SARS-CoV2 virus infection group (DMSO-S, DMSO-S) decreased.

In the positive control group treated with remdesivir, cell survival increased in a concentration-dependent manner when compared to the control group SARS-CoV2 virus infection group (DMSO-S, DMSO-S).

Compared to the control group (DMSO-S) (cell viability of 66.52±2.28%), cell viability increased in GM90129 (77.68±3.6%), GM90228 (74.31±3.43%) or BIA (77.94±5.51%) at 24 hours after treatment. Data were represented as mean±SD. Comparisons between groups were made using Dunnett's test with post-test (*, P<0.05 compared to SARS-CoV2-DMSO treatment group (DMSO-S), #, P<0.05 compared to control DMSO group (DMSO).

What is claimed is:

1. A pharmaceutical composition for preventing or treating BI-1 related diseases comprising compounds of Formula 1 or pharmaceutically acceptable salts, hydrates or solvates thereof, 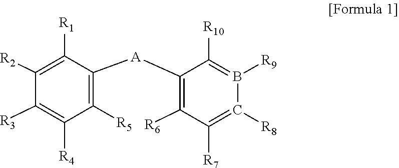
wherein A is the following Formula 1-1, 1-2, or 1-3, 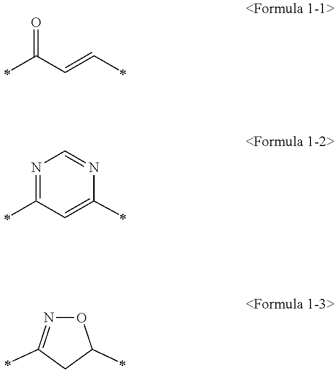
(* of formula 1-1 to 1-3 are connected to the phenyl group of formula 1)
B and C are C, or N, respectively,
the R₁ to R₁₀ are respectively H; NH₂; NO2; OH; OR (R is a linear, branched alkyl, alkenyl, or alkynyl of C₁ to C₁₀); halogen atoms; CN; Alkyl halides of C₁ to C₃; a linear, branched alkyl, alkenyl, or alkynyl group of C1 to C10; —NH—C(O)—ORa (Ra is a linear, branched alkyl, alkenyl , or alkynyl of C₁ to C₁₀ IM); —C(O)—NH—RA (Ra is a linear, branched alkyl, alkenyl, or alkynyl of C₁ to C₁₀); —NH₂HCl; —C(O)OH; And substituents having oxide; One or more selected from the group consisting of,
If B or C is N, R₈ and R₉ are hydrogen,
when B or C is C, R₈ and R₉ can be connected to form a phenyl ring, the substituent having the jade core is represented by the following Formula 1-4, 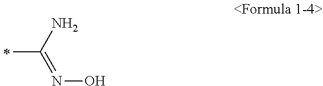
(* of Formula 1-4 is where substituents R₁ to R₁₀ of Formula 1 are substituted).

2. A pharmaceutical composition for preventing or treating mTORC2 related diseases comprising compounds of Formula 1 or pharmaceutically acceptable salts, hydrates or solvates thereof, 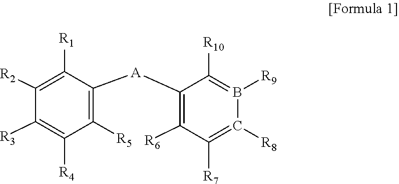
wherein A is the following Formula 1-1, 1-2, or 1-3, 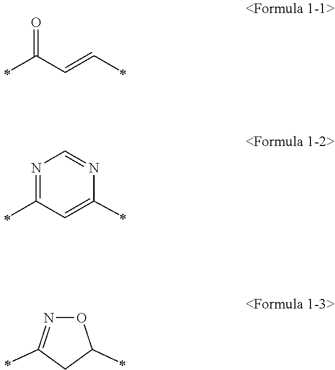
(* of formula 1-1 to 1-3 are connected to the phenyl group of formula 1)
B and C are C, or N, respectively,
the R₁ to R₁₀ are respectively H; NH₂; NO2; OH; OR (R is a linear, branched alkyl, alkenyl, or alkynyl of C₁ to C₁₀); halogen atoms; CN; Alkyl halides of C₁ to C₃; a linear, branched alkyl, alkenyl, or alkynyl group of C₁ to C₁₀; —NH—C(O)—ORa (Ra is a linear, branched alkyl, alkenyl , or alkynyl of C₁ to C₁₀ IM); —C(O)—NH—RA (Ra is a linear, branched alkyl, alkenyl, or alkynyl of C₁ to C₁₀); —NH₂ HCl; —C(O)OH; And substituents having oxide; One or more selected from the group consisting of,
if B or C is N, R 8 and R9 are hydrogen,
when B or C is C, R 8 and R9 can be connected to each other to form a phenyl ring,
the substituent having the jade core is represented by the following Formula 1-1, 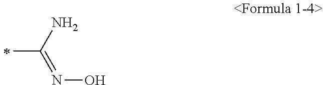
(* of Formula 1-4 is where substituents R₁ to R₁₀ of Formula 1 are substituted).

3. A pharmaceutical composition for preventing or treating AKT-related diseases comprising compounds of Formula 1 or pharmaceutically acceptable salts, hydrates or solvates thereof, 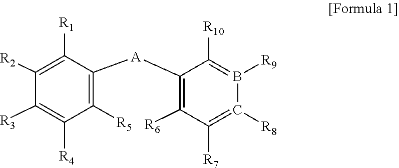
wherein A is the following Formula 1-1, 1-2, or 1-3, 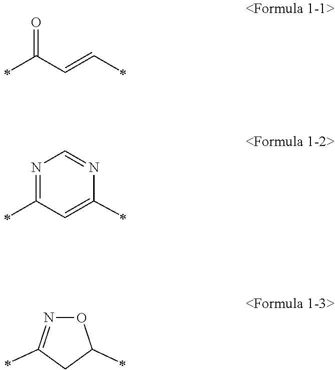
(* of formula 1-1 to 1-3 are connected to the phenyl group of formula 1)
B and C are C, or N, respectively,
the R₁ to R₁₀ are respectively H; NH₂; NO2; OH; OR (R is a linear, branched alkyl, alkenyl, or alkynyl of C₁ to C₁₀); halogen atoms; CN; Alkyl halides of C1 to C₃; a linear, branched alkyl, alkenyl, or alkynyl group of C1 to C10; —NH—C(O)—ORa (Ra is a linear, branched alkyl, alkenyl , or alkynyl of C₁ to C₁₀ IM); —C(O)—NH—RA (Ra is a linear, branched alkyl, alkenyl, or alkynyl of C₁ to C₁₀); —NH₂HCl; —C(O)OH; And one or more selected from the group consisting of substituents having a jade core,
if B or C is N, R₈ and R₉ are hydrogen,
when B or C is C, R₈ and R₉ can be connected to each other to form a phenyl ring, the substituent having the jade core is represented by the following Formula 1-4, 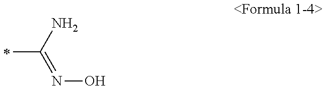
(* of Formula 1-4 is where substituents R1 to R₁₀ of Formula 1 are substituted).

4. The pharmaceutical composition of claim 1, wherein B and C are C, respectively, and A is Formula 1-1, Formula 1-2 or Formula 1-3.

5. The pharmaceutical composition of claim 1, wherein B or C is N and A is Formula 1-1, a pharmaceutical composition.

6. The pharmaceutical composition of claim 1, wherein if the compound
(1) 2E-1-2-Aminophenyl-3-3-nitrophenyl-2-propen-1-one (BIA).
(2) 2-(5-(3-(trifluoromethyl)phenyl)isoxazol-3-yl)aniline (GM-90340);
(3) 2-(5-(3-(trifluoromethyl)phenyl)isoxazol-3-yl)aniline (GM-90339);
(4) (Z)-3-((E)-3-(2-aminophenyl)-3-oxoprop-1-en-1-yl)-N′-hydroxybenzimidamide (GM-90338).
(5) 3-(3-(2-aminophenyl)isoxazol-5-yl)benzonitrile (GM-90337);
(6) (E)-1-(2-amino-4,5-dimethoxyphenyl)-3-(3-bromo-5-hydroxyphenyl)prop-2-en-1-one (GM-90321);
(7) (E)-1-(2-amino-4,5-dimethoxyphenyl)-3-(3-(trifluoromethyl)phenyl)prop-2-en-1-one (GM-90320);
(8) (E)-3-(3-(2-amino-4,5-dimethoxyphenyl)-3-oxoprop-1-en-1-yl)benzonitrile (GM-90319);
(9) (E)-1-(2-aminophenyl)-3-(3-fluorophenyl)prop-2-en-1-one (GM-90318);
(10) (E)-1-(2-aminophenyl)-3-(3-chlorophenyl)prop-2-en-1-one (GM-90317);
(11) (E)-1-(2-aminophenyl)-3-(3,5-difluoro-4-hydroxyphenyl)prop-2-en-1-one (GM-90316);
(12) (E)-1-(2-aminophenyl)-3-(3-aminophenyl)prop-2-en-1-one hydrochloride (GM-90315);
(13) (E)-3-(3-(2-aminophenyl)-3-oxoprop-1-en-1-yl)-N-methyl benzamide (GM-90300);
(14) tert-butyl (E)-(3-(3-(2-aminophenyl)-3-oxoprop-1-en-1-yl)phenyl) carbamate (GM-90299);
(15) (E)-1-(2-amino-5-fluorophenyl)-3-(3-nitrophenyl)prop-2-en-1-one (GM-90298);
(16) (E)-1-(2-amino-4-methoxyphenyl)-3-(3-nitrophenyl)prop-2-en-1-one (GM-90297);
(17) (E)-3-(3-(2-aminophenyl)-3-oxoprop-1-en-1-yl)benzoic acid (GM-90296);
(18) (E)-1-(2-aminophenyl)-3-(3-ethoxyphenyl)prop-2-en-1-one (GM-90295);
(19) (E)-1-(2-aminophenyl)-3-(pyridin-3-yl)prop-2-en-1-one (GM-90285);
(20) (E)-1-(2-aminophenyl)-3-(rn-tolyl)prop-2-en-1-one (GM-90284);
(21) (E)-1-(2-aminophenyl)-3-(3-(trifluoromethyl)phenyl)prop-2-en-1-one (GM-90283);
(22) (E)-3-(3-(2-aminophenyl)-3-oxoprop-1-en-1-yl)benzonitrile (GM-90282);
(23) (E)-1-(2-aminophenyl)-3-(3-bromo-5-hydroxyphenyl)prop-2-en-1-one (GM-90281);
(24) (E)-1-(2-amino-4,5-dimethoxyphenyl)-3-(3-methoxyphenyl)prop-2-en-1-one (GM-90256);
(25) (E)-1-(2-amino-4,5-dimethoxyphenyl)-3-(3-bromophenyl)prop-2-en-1-one (GM-90255);
(26) (E)-1-(2-amino-4,5-dimethoxyphenyl)-3-(3-nitrophenyl)prop-2-en-1-one (GM-90254);
(27) (E)-1-(2-aminophenyl)-3-(4-(tert-butyl)phenyl)prop-2-en-1-one (GM-90243);
(28) 1-(2-aminophenyl)-3-(2-ethoxy-5-nitrophenyl)-3-hydroxypropan-1-one (GM-90230);
(29) (E)-1-(2-aminophenyl)-3-(2-ethoxy-5-nitrophenyl)prop-2-en-1-one (GM-90229);
(30) (E)-1-(2-aminophenyl)-3-(pyridin-4-yl)prop-2-en-1-one (GM-90228);
(31) (E)-1-(2-aminophenyl)-3-(p-tolyl)prop-2-en-1-one (GM-90227);
(32) (E)-1-(2-aminophenyl)-3-(4-(trifluoromethyl)phenyl)prop-2-en-1-one (GM-90226);
(33) (E)-1-(2-aminophenyl)-3-(naphthalen-2-yl)prop-2-en-1-one (GM-90225);
(34) (E)-1-(2-aminophenyl)-3-(2-ethoxy-4-fluorophenyl)prop-2-en-1-one (GM-90224);
(35) (E)-1-(2-aminophenyl)-3-(2,4-difluorophenyl)prop-2-en-1-one (GM-90223);
(36) (E)-1-(2-aminophenyl)-3-(4-(trifluoromethoxy)phenyl)prop-2-en-1-one (GM-90222);
(37) (E)-1-(2-hydroxyphenyl)-3-(3-nitrophenyl)prop-2-en-1-one (GM-90135);
(38) (E)-1-(2-methoxyphenyl)-3-(3-nitrophenyl)prop-2-en-1-one (GM-90134);
(39) (E)-1-(2-aminophenyl)-3-(4-nitrophenyl)prop-2-en-1-one (GM-90133);
(40) (E)-1-(2-aminophenyl)-3-(3-bromophenyl)prop-2-en-1-one (GM-90132);
(41) (E)-1-(2-aminophenyl)-3-(3-methoxyphenyl)prop-2-en-1-one (GM-90131);
(42) (E)-1-(2-aminophenyl)-3-phenylprop-2-en-1-one (GM-90130);
(43) (E)-1-(2-aminophenyl)-3-(3-nitrophenyl)prop-2-en-1-one (GM-90129); and
(44) (E)-3-(3-nitrophenyl)-1-phenylprop-2-en-1-one (GM-90128);
consists of a pharmaceutical composition, is characterized in that it is selected from the gin group.

7. The pharmaceutical composition of claim 1, wherein the compound of Formula 1 is in the form of a racemate, an enantiomer, a diastereomer, or a diastereomer.

8. A method of preventing or treating BI-1 related diseases using the pharmaceutical composition of claim 1,
wherein the BI-1 related diseases include liver diseases such as liver ischemic reperfusion injury, chronic hepatitis, carbon tetrachloride-induced liver damage, tumor formation, cancer, respiratory diseases with asthma and COPD diseases, viral diseases, autoimmune diseases, Neurological disease, characterized in that it is selected from the group consisting of insulin resistance, pharmaceutical composition.

9. The method of claim 8,
wherein the cancers include, lung cancer, lung adenocarcinoma, pancreatic cancer, colon cancer, colorectal cancer, myeloid leukemia, thyroid cancer, myelotype dysmorphic syndrome (MDS), bladder carcinoma, epidermal carcinoma, melanoma, breast cancer, prostate cancer, head and neck cancer, Uterine cancer, ovarian cancer, brain cancer, stomach cancer, laryngeal cancer, esophageal cancer, bladder cancer, oral cancer, nasopharyngeal cancer, cancer of mesenchymal origin, fibrosarcoma, teratoma carcinoma, neuroblastoma, kidney carcinoma, liver cancer, non-Pharmaceutical composition, characterized in that it is selected from the group consisting of Hodgkin's lymphoma, multiple myeloma, and undifferentiated thyroid cancer.

10. A method of preventing or treating mTORC2 related diseases using the pharmaceutical composition of claim 2,
wherein the mTORC2 related disease is selected from the group consisting of metabolic diseases such as type 2 diabetes, cancer tumors, lung fibrosis, asthma, viral infections, respiratory diseases including COPD, and systemic lupus erythematosus, pharmaceutical composition.

11. A method of preventing or treating AKT-related diseases using the pharmaceutical composition of claim 3,
wherein he AKT related disease is selected from the group consisting of cancer, diabetes, cardiovascular disease, inflammatory disease, asthma, and viral infectious disease, wherein the pharmaceutical composition.

12. The method of claim 11, wherein the asthma comprises a general allergic asthma or steroid resistant asthma.

13. The method of claim 11, wherein the viral infectious agent comprises a COVID19, COVID 19 variant, or similar coronavirus infection of the MERS virus.

14. The pharmaceutical composition of claim 1, wherein the pharmaceutical composition is controlled by the antagonism of BI-1.

15. A method of preventing or treating BI-1 related diseases using the pharmaceutical composition of claim 14, wherein the antagonism of the BI-1 inhibits the activity of mTOR or reduces phosphorylation of AKT or S6K.

16. A dietary supplement composition for preventing, improving or alleviating symptoms of BI-related diseases, mTORC2, or AKT-related diseases comprising the compounds of Formula 1, hydrates thereof, or solvates thereof of the pharmaceutical composition of claim 1.

17. The dietary supplement composition of claim 16, wherein the related disease is selected from the group consisting of cancer, asthma, and coronavirus infection.

18. A method of inhibiting BI-1, mTORC2, or AKT using the compound of Formula 1, a salt thereof, a hydrate thereof, or a solvate thereof of the pharmaceutical composition of claim 1.

19. The method of claim 18, wherein the method is performed in vitro.

20. A composition for inhibiting BI-1, mTORC2, or AKT inhibition comprising the compound of Formula 1, a salt thereof, a hydrate thereof, a solvate thereof of the pharmaceutical composition of claim 1.

21. A kit comprising the composition of claim 20.

22. An antagonist of BI-1 comprising the compounds of Formula 1 and salts, hydrates, or solvates thereof of the pharmaceutical composition of claim 1.
